(19) United States
(12) Patent Application Publication
(10) Pub. No.: US 2020/0318106 A1
(43) Pub. Date: Oct. 8, 2020

(54) CONTROLLING PHENOTYPE OF ORGANISMS WITH CRISPR/CAS GENE TARGETING

(71) Applicant: Bio-Rad Laboratories, Inc., Hercules, CA (US)

(72) Inventors: Alex Michael WARD, El Cerrito, CA (US); Molly Mo-Yin LEUNG, El Cerrito, CA (US); Jean Frances SPAGNOLO-SMITH, Fairfield, CA (US); George CHENAUX, Oakland, CA (US)

(21) Appl. No.: 16/955,682

(22) PCT Filed: Dec. 20, 2018
(86) PCT No.: PCT/US2018/066908

§ 371 Date: Jun. 18, 2020

Related U.S. Application Data
(60) Provisional application No. 62/610,040, filed on Dec. 22, 2017.

(51) Int. Cl. C12N 15/10; C12N 9/22; C12N 15/113; C12N 15/72

(52) U.S. Cl. CPC C12N 15/1086; C12N 9/22; C12N 2800/80; C12N 15/72; C12N 15/113

(57) ABSTRACT

Engineered microbial organisms for altering gene expression are provided. In some embodiments, the engineered microbial organism comprises: one or more heterologous polynucleotide sequence comprising a phenotype coding sequence operably linked to a promoter; a heterologous polynucleotide sequence comprising an inducible promoter operably linked to a polynucleotide encoding a Cas nuclease; and a heterologous polynucleotide sequence comprising a guide RNA (gRNA) that targets the phenotype coding sequence.

### CROSS-REFERENCE TO RELATED APPLICATIONS

This application claims priority to U.S. Provisional Patent Application No. 62/610,040, filed Dec. 22, 2017, the entire content of which is incorporated by reference herein.

### BACKGROUND OF THE INVENTION

Genomic editing technologies can be used to introduce modifications in nucleic acid sequences in a sequence-specific manner. One such technology utilizes clustered regularly interspaced short palindromic repeats (CRISPR), which are segments of prokaryotic DNA that contain short repetitive nucleotide sequences, and CRISPR-associated (Cas) nucleases that induce a double-stranded break (DSB) in nucleic acid. The Cas nuclease can specifically induce a DSB at a target genomic locus through the use of a guide nucleic acid that targets the genomic locus. Following the induction of a DSB in the nucleic acid, repair of the DSB can occur via non-homologous end joining (NHEJ), alternative-end joining (A-EJ), and/or homology-directed repair (HDR). The CRISPR/Cas system can be used to introduce modifications that disrupt expression of a nucleic acid and/or to repair an existing mutation in a nucleic acid sequence in order to restore expression of the nucleic acid.

### BRIEF SUMMARY OF THE INVENTION

In one aspect, engineered cells and microbial organisms are provided. In some embodiments, an engineered cell comprises:

an endogenous gene or genomic region to be targeted for gene alteration, or one or more heterologous polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype; and

(a) a heterologous polynucleotide sequence comprising a promoter operably linked to a polynucleotide encoding a Cas nuclease and a heterologous polynucleotide sequence comprising a guide RNA (gRNA) that targets the endogenous gene, genomic region, or phenotype coding sequence, and/or (b) a heterologous polynucleotide sequence comprising a promoter operably linked to a polynucleotide encoding lambda red.

In some embodiments, an engineered cell that comprises an endogenous gene or genomic region to be targeted for gene alteration, or one or more heterologous polynucleotide sequences comprising a phenotype coding sequence operably linked to a promoter, comprises a heterologous polynucleotide sequence comprising a promoter operably linked to a polynucleotide encoding a Cas nuclease and a heterologous polynucleotide sequence comprising a gRNA that targets the endogenous gene, genomic region, or phenotype coding sequence. In some embodiments, an engineered cell that comprises an endogenous gene or genomic region to be targeted for gene alteration, or one or more heterologous polynucleotide sequences comprising a phenotype coding sequence operably linked to a promoter, comprises a heterologous polynucleotide sequence comprising a promoter operably linked to a polynucleotide encoding lambda red. In some embodiments, an engineered cell that comprises an endogenous gene or genomic region to be targeted for gene alteration, or one or more heterologous polynucleotide sequences comprising a phenotype coding sequence operably linked to a promoter, comprises a heterologous polynucleotide sequence comprising a promoter operably linked to a polynucleotide encoding a Cas nuclease, a heterologous polynucleotide sequence comprising a gRNA that targets the endogenous gene, genomic region, or phenotype coding sequence, and a heterologous polynucleotide sequence comprising a promoter operably linked to a polynucleotide encoding lambda red. In some embodiments, the engineered cell further comprises a donor DNA sequence.

In some embodiments, an engineered cell comprises an endogenous gene or genomic region to be targeted for gene alteration. In some embodiments, the endogenous gene or genomic region to be targeted for gene alteration is a functional gene or genomic region (e.g., produces a detectable phenotype) and the donor DNA sequence is a sequence that disrupts the functional gene or genomic region. In some embodiments, the endogenous gene or genomic region to be targeted for gene alteration is a functional gene or genomic region (e.g., produces a detectable phenotype) and the donor DNA sequence is a sequence that replaces the functional gene or genomic region with a different functional gene or different functional genomic region. In some embodiments, the endogenous gene or genomic region to be targeted is a disrupted gene or genomic region (e.g., a disruption that prevents expression of a detectable phenotype) and wherein the donor DNA sequence is a sequence that restores the function of the gene or genomic region.

In some embodiments, an engineered cell comprises one or more heterologous polynucleotide sequences comprising a phenotype coding sequence operably linked to a promoter. In some embodiments, an engineered cell comprises:

one or more heterologous polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype.

In some embodiments, the engineered cell comprises a phenotype coding sequence that encodes a functional protein having a detectable phenotype (e.g., a chromogenic coding sequence that encodes a functional chromogenic protein). In some embodiments, the engineered cell comprises a phenotype coding sequence that comprises an engineered disruption in a sequence encoding a protein or promoter region that prevents expression of the detectable phenotype (e.g., a broken chromogenic gene that prevents expression of a chromogenic protein).

In some embodiments, an engineered cell comprises:

one or more heterologous polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype;

a heterologous polynucleotide sequence comprising a promoter operably linked to a polynucleotide encoding a Cas nuclease;

a heterologous polynucleotide sequence comprising a guide RNA (gRNA) that targets the phenotype coding sequence.

In some embodiments, the cell comprises:

a heterologous polynucleotide sequence comprising a phenotype coding sequence that either (i) encodes a functional protein having a detectable phenotype, or (ii) comprises an engineered disruption in a sequence encoding a protein that prevents expression of the detectable phenotype;

a heterologous polynucleotide sequence comprising a promoter operably linked to a polynucleotide encoding a Cas nuclease;

a heterologous polynucleotide sequence comprising a gRNA that targets the chromogenic coding sequence; and

a heterologous polynucleotide sequence comprising a homologous donor DNA sequence.

In some embodiments, the phenotype coding sequence encodes a functional protein having a detectable phenotype (e.g., a functional chromogenic or fluorescent protein, e.g., coral fluorescent protein). In some embodiments, the phenotype coding sequence comprises an engineered disruption in a sequence encoding a protein or promoter region that prevents expression of the detectable phenotype (e.g., a broken gene such as a broken chromogenic gene or broken fluorescent protein, e.g., a coral fluorescent protein). In some embodiments, the cell comprises a heterologous polynucleotide sequence comprising a homologous donor DNA sequence (e.g., for fixing a broken gene).

In some embodiments, an engineered cell comprises:

a first heterologous polynucleotide sequence comprising a first phenotype coding sequence operably linked to a first promoter, wherein the first phenotype coding sequence either (i) encodes a functional first protein having a first detectable phenotype, or (ii) comprises an engineered disruption in a sequence encoding a first protein that prevents expression of the first detectable phenotype;

a second heterologous polynucleotide sequence comprising a second phenotype coding sequence operably linked to a second promoter, wherein the second phenotype coding sequence either (i) encodes a functional second protein having a second detectable phenotype, or (ii) comprises an engineered disruption in a sequence encoding a second protein that prevents expression of the second detectable phenotype;

a third heterologous polynucleotide sequence comprising a third promoter operably linked to a polynucleotide encoding a Cas nuclease (e.g., Cas9);

a fourth heterologous polynucleotide sequence comprising a first gRNA that targets the first phenotype coding sequence; and

a fifth heterologous polynucleotide sequence comprising a second gRNA that targets the second phenotype coding sequence.

In some embodiments, the detectable phenotype (e.g., each of the first detectable phenotype and the second detectable phenotype) is a detectable color, fluorescence, scent, enzymatic activity, or morphology. In some embodiments, the detectable phenotype (e.g., each of the first detectable phenotype and the second detectable phenotype) is lethality. In some embodiments, the detectable phenotype (e.g., each of the first detectable phenotype and the second detectable phenotype) is gain or loss of resistance to an antibiotic.

In some embodiments, the detectable phenotype is a detectable fluorescence, and wherein the phenotype coding sequence either (i) encodes a functional fluorescent protein, or (ii) comprises an engineered disruption in a sequence encoding a protein that prevents expression of the fluorescent protein. In some embodiments, the fluorescent protein is a coral fluorescent protein.

In some embodiments, the first detectable phenotype and the second detectable phenotype is a detectable color and/or fluorescence, and:

the first chromogenic coding sequence either (i) encodes a functional first chromogenic protein or (ii) comprises an engineered disruption in a sequence encoding a first chromogenic protein that prevents expression of the first chromogenic protein; and

the second chromogenic coding sequence either (i) encodes a functional second chromogenic protein or (ii) comprises an engineered disruption in a sequence encoding a second chromogenic protein that prevents expression of the second chromogenic protein.

In some embodiments, one or more polynucleotide sequences as disclosed herein (e.g., a polynucleotide sequence comprising a phenotype coding sequence, a polynucleotide sequence comprising a promoter operably linked to a polynucleotide encoding a Cas nuclease, or a polynucleotide sequence comprising a gRNA that targets the phenotype coding sequence) further comprises an auxotrophic, antibiotic, or other selectable marker. In some embodiments, one or more of the first polynucleotide sequence, second polynucleotide sequence, third polynucleotide sequence, fourth polynucleotide sequence, or fifth polynucleotide sequence further comprises an auxotrophic, antibiotic, or other selectable marker. In some embodiments, wherein a fourth heterologous polynucleotide sequence comprises a first gRNA that targets the first phenotype coding sequence and a fifth polynucleotide sequence comprises a second gRNA that targets the second phenotype coding sequence, the fourth polynucleotide sequence further comprises a first auxotrophic, antibiotic, or other selectable marker and the fifth polynucleotide sequence further comprises a second auxotrophic, antibiotic, or other selectable marker, wherein the first auxotrophic, antibiotic, or other selectable marker and the second auxotrophic, antibiotic, or other selectable marker are different markers. In some embodiments, the first auxotrophic, antibiotic, or other selectable marker and the second auxotrophic, antibiotic, or other selectable marker are the same marker.

In some embodiments, the cell comprises a heterologous polynucleotide comprising a homologous donor polynucleotide sequence that is used as a template for repair of the Cas cleavage site. In some embodiments, the homologous donor polynucleotide sequence is double-stranded DNA (dsDNA) or single-stranded DNA (ssDNA). In some embodiments, the heterologous polynucleotide sequence comprising the homologous donor DNA sequence comprises a mutation that prevents expression of the chromogenic protein. In some embodiments, the heterologous polynucleotide sequence comprising the homologous donor DNA sequence comprises a mutation that modifies expression of the chromogenic protein from a first color to a second color. In some embodiments, the heterologous polynucleotide sequence comprising the homologous donor DNA sequence comprises a mutation that repairs a polynucleotide sequence comprising an engineered disruption (e.g., a broken gene) and restores expression of a functional chromogenic protein. In some embodiments, one or more polynucleotide sequences as disclosed herein (e.g., a polynucleotide sequence comprising a phenotype coding sequence, a polynucleotide sequence comprising a promoter operably linked to a polynucleotide encoding a Cas nuclease, or a polynucleotide sequence comprising a gRNA that targets the phenotype coding sequence) further comprises a homologous donor polynucleotide sequence, e.g., a double-stranded DNA (dsDNA) or single-stranded DNA (ssDNA) donor sequence. In some embodiments, the fourth polynucleotide sequence and/or the fifth polynucleotide sequence further comprises a homologous donor polynucleotide sequence. In some embodiments, wherein a fourth heterologous polynucleotide sequence comprises a first gRNA that targets the first phenotype coding sequence and a fifth polynucleotide sequence comprises a second gRNA that targets the second phenotype coding sequence, the fourth polynucleotide sequence and/or the fifth polynucleotide sequence further comprises a homologous donor polynucleotide sequence.

In some embodiments, a polynucleotide sequence comprising a gRNA that targets an endogenous gene, genomic region, or phenotypic coding sequence (e.g., a chromogenic coding sequence) is operably linked to the polynucleotide sequence comprising the promoter operably linked to the polynucleotide encoding a Cas nuclease. In some embodiments, a polynucleotide sequence comprising a gRNA that targets an endogenous gene, genomic region, or phenotypic coding sequence (e.g., a chromogenic coding sequence) and a polynucleotide sequence comprising a homologous donor DNA sequence are operably linked to the polynucleotide sequence comprising the promoter operably linked to the polynucleotide encoding a Cas nuclease.

In some embodiments, a promoter is a constitutively active promoter. In some embodiments, one or more of the first promoter, second promoter, and third promoter are constitutively active promoters. In some embodiments, both the first promoter and the second promoter are constitutively active promoters.

In some embodiments, a promoter is an inducible promoter. In some embodiments, one or more of the first promoter, second promoter, and third promoter are inducible promoters. In some embodiments, both the first promoter and the second promoter are inducible promoters. In some embodiments, the inducible promoter is a galactose-inducible promoter. In some embodiments, the inducible promoter is an arabinose-inducible promoter. In some embodiments, the inducible promoter is a rhamnose-inducible promoter.

In some embodiments, the Cas nuclease is a Cas9 nuclease. In some embodiments, the Cas nuclease is a Cas9 nuclease from Streptococcus pyogenes (SpCas9 nuclease). In some embodiments, the polynucleotide encoding the Cas nuclease is optimized for expression in the cell (e.g., eukaryotic or prokaryotic cell). In some embodiments, the Cas nuclease is a Cas9 nuclease that is codon optimized for expression in a yeast cell. In some embodiments, the Cas nuclease is a Cas9 nuclease that is codon optimized for expression in an E. coli cell. In some embodiments, the Cas nuclease is a destabilized variant of Cas9.

In some embodiments, the cell comprises a heterologous polynucleotide sequence comprising a chromogenic coding sequence that encodes a functional chromogenic protein (e.g., a GFP protein), and expression of the Cas nuclease and repair of the Cas cleavage results in the heterologous polynucleotide sequence having a mutation that prevents the expression of the chromogenic protein (e.g., prevents GFP expression). In some embodiments, the cell comprises a heterologous polynucleotide sequence comprising a chromogenic coding sequence that encodes a functional chromogenic protein (e.g., a GFP protein), and expression of the Cas nuclease and repair of the Cas cleavage results in the heterologous polynucleotide sequence having one or more mutations that result in the expression of a second chromogenic protein (e.g., a YFP or BFP instead of GFP).

In some embodiments, the first chromogenic coding sequence encodes a functional first chromogenic protein and/or the second chromogenic coding sequence encodes a functional second chromogenic protein. In some embodiments, the first coding sequence comprises an engineered disruption in a sequence encoding a first chromogenic protein that prevents expression of the first chromogenic protein or its promoter, and/or the second coding sequence comprises an engineered disruption in a sequence encoding a second chromogenic protein that prevents expression of the second chromogenic protein or its promoter. In some embodiments, the first chromogenic coding sequence encodes a functional first chromogenic protein and the second coding sequence comprises an engineered disruption in a sequence encoding a second chromogenic protein that prevents expression of the second chromogenic protein or its promoter. In some embodiments, the first coding sequence comprises an engineered disruption in a sequence encoding a first chromogenic protein that prevents expression of the first chromogenic protein or its promoter, and the second chromogenic coding sequence encodes a functional second chromogenic protein.

In some embodiments, one or more of the polynucleotide sequences are in the same expression cassette or plasmid. In some embodiments, at least two polynucleotide sequences are in separate expression cassettes or plasmids. In some embodiments, the first polynucleotide sequence and the second polynucleotide are in the same expression cassette or plasmid. In some embodiments, the first polynucleotide sequence and the second polynucleotide are in separate expression cassettes or plasmids. In some embodiments, two or more (e.g., two, three, four, or five) of each the first polynucleotide sequence, the second polynucleotide sequence, the third polynucleotide sequence, the fourth polynucleotide sequence, and the fifth polynucleotide sequence are in the same expression cassette or plasmid. In some embodiments, each of the first polynucleotide sequence, the second polynucleotide sequence, the third polynucleotide sequence, the fourth polynucleotide sequence, and the fifth polynucleotide sequence is in a separate expression cassette or plasmid.

In some embodiments, the cell is a microbial cell. In some embodiments, the cell is a prokaryotic cell. In some embodiments, the cell is a prokaryotic cell selected from the group consisting of bacteria and archaebacteria. In some embodiments, the cell is a protist cell. In some embodiments, the cell is a eukaryotic cell. In some embodiments, the eukaryotic cell is a yeast cell, fungal cell, mammalian cell, insect cell, nematode cell, or plant cell. In some embodiments, the cell is a bacterial cell, e.g., an E coli cell. In some embodiments, the cell is a nematode cell, e.g., C. elegans.

In some embodiments, the third polynucleotide sequence comprises a polynucleotide encoding a Cas9 nuclease that is from S. pyogenes or that is codon optimized for expression in the particular cell or organism in which it is expressed. In some embodiments, the cell is a yeast cell and the third polynucleotide sequence comprises a polynucleotide encoding a Cas9 nuclease that is codon optimized for expression in yeast.

In another aspect, engineered microbial organisms comprising an engineered cell as described herein are provided. In some embodiments, the engineered organism is eukaryotic (e.g., an engineered yeast or an engineered plant). In some embodiments, the engineered organism is prokaryotic (e.g., an engineered bacterium). In some embodiments, the engineered organism is in lyophilized form.

In another aspect, kits are provided. In some embodiments, the kit comprises an engineered cell or engineered microbial organism as described herein, and further comprises one or more reagents comprising culture medium, selective medium, media supplements, solid plating medium, plates, tubes, loops, or other plasticware. In some embodiments, the kit further comprises one or more heterologous polynucleotide sequences comprising a homologous donor DNA (e.g., double stranded DNA or single stranded DNA). In some embodiments, the kit further comprises an inducer for inducing expression of an inducible promoter (e.g., galactose for a galactose-inducible promoter, arabinose for an arabinose-inducible promoter, or rhamnose for a rhamnose-inducible promoter). In some embodiments, the kit further comprises one or more reagents for detecting the genotype of the engineered cell or engineered microbial organism, wherein the one or more reagents comprise DNA polymerases, primers, dNTPs, restriction enzymes, or buffers. In some embodiments, the kit further comprises one or more counterselection agents or selection agents for an auxotrophic, antibiotic, or other selectable marker as described herein.

In some embodiments, the kit comprises an engineered cell or organism comprising one or more heterologous polynucleotide sequences comprising a phenotype coding sequence that may be operably linked to a promoter, wherein the phenotype coding sequence either (i) encodes a functional protein having a detectable phenotype, or (ii) comprises an engineered disruption in a sequence encoding a protein that prevents expression of the detectable phenotype; and further comprises one or more of a heterologous polynucleotide sequence comprising a promoter operably linked to a polynucleotide encoding a Cas nuclease, a heterologous polynucleotide sequence comprising a gRNA that targets the phenotype coding sequence, and a heterologous polynucleotide sequence comprising a homologous donor DNA sequence.

In still another aspect, methods of altering gene expression in a cell are provided. In some embodiments, the method comprises:

culturing an engineered cell or engineered microbial organism as described herein to form a population of engineered cells or engineered microbial organisms, wherein the culturing is performed under conditions that result in expression of the Cas nuclease in at least one engineered cell or one engineered microbial organism, wherein the Cas nuclease cleaves a phenotype coding sequence;

thereby altering gene expression in at least one engineered cell or one engineered microbial organism.

In some embodiments, a promoter is operably linked to a polynucleotide encoding a

Cas nuclease is an inducible promoter and the culturing is in the presence of an inducer to induce expression of the Cas nuclease in at least one engineered cell. In some embodiments, the promoter is a galactose-inducible promoter and the inducer is galactose. In some embodiments, the promoter is an arabinose-inducible promoter and the inducer is arabinose. In some embodiments, the promoter is a rhamnose-inducible promoter and the inducer is rhamnose.

In some embodiments, a method of altering gene expression comprises:

culturing an engineered cell or engineered microbial organism as described herein to form a population of engineered cells, wherein the culturing is performed under conditions that result in expression of the lambda red in at least one engineered cell, wherein the lambda red catalyzes the homologous recombination of a donor DNA sequence at an endogenous gene, genomic region, or phenotype coding sequence in the engineered cell;

thereby altering gene expression in at least one engineered cell.

In some embodiments, the lambda red is under the control of an inducible promoter, and wherein the culturing is in the presence of an inducer to induce expression of the lambda red in at least one engineered cell. In some embodiments, the promoter is a galactose-inducible promoter and the inducer is galactose. In some embodiments, the promoter is an arabinose-inducible promoter and the inducer is arabinose. In some embodiments, the promoter is a rhamnose-inducible promoter and the inducer is rhamnose.

In some embodiments, the method of altering gene expression comprises expressing both the Cas nuclease and the lambda red system in at least one engineered cell. In some embodiments, both the Cas nuclease and the lambda red system are under the control of an inducible promoter. In some embodiments, expression of both the Cas nuclease and the lambda red system are induced by the same inducer. In some embodiments, expression of the Cas nuclease and the lambda red system are induced by different inducers.

In some embodiments, prior to the culturing step, the method comprises transforming or transfecting a cell with one or more heterologous polynucleotide sequences as disclosed herein to produce the engineered cell. In some embodiments, the transforming comprises chemical transformation, transformation by electroporation, or transformation by silicon-carbide whiskers.

In some embodiments, the method further comprises screening the population of engineered cells to identify at least one engineered cell that exhibits a change in phenotype as compared to the phenotype of the engineered cell prior to the culturing step.

In some embodiments, the engineered cell or microbial organism comprises a heterologous polynucleotide sequence comprising a chromogenic coding sequence that encodes a functional chromogenic protein, a heterologous polynucleotide encoding a Cas nuclease, a heterologous polynucleotide sequence comprising a gRNA that targets the chromogenic coding sequence, and a heterologous polynucleotide sequence comprising a homologous donor DNA sequence that comprises a mutation that prevents expression of the chromogenic protein, and the method further comprises: culturing the population of engineered cells under conditions that result in expression of the gRNA in at least one engineered cell; and screening the population of engineered cells to identify at least one engineered cell that does not express the chromogenic protein.

In some embodiments, the engineered cell or microbial organism comprises a heterologous polynucleotide sequence comprising a chromogenic coding sequence that encodes a functional chromogenic protein, a heterologous polynucleotide encoding a Cas nuclease, a heterologous polynucleotide sequence comprising a gRNA that targets the chromogenic coding sequence, and a heterologous polynucleotide sequence comprising a homologous donor DNA sequence that comprises a mutation that modifies expression of the chromogenic protein from a first color to a second color, and the method further comprises: culturing the population of engineered cells under conditions that result in expression of the gRNA in at least one engineered cell; and screening the population of engineered cells to identify at least one engineered cell that expresses the second color.

In some embodiments, the engineered cell or microbial organism comprises a heterologous polynucleotide sequence comprising a chromogenic coding sequence that comprises an engineered disruption in a sequence encoding a chromogenic protein that prevents expression of the functional chromogenic protein, a heterologous polynucleotide encoding a Cas nuclease, a heterologous polynucleotide sequence comprising a gRNA that targets the chromogenic coding sequence, and a heterologous polynucleotide sequence comprising a homologous donor DNA sequence for repairing the engineered disruption, and the method further comprises: culturing the population of engineered cells under conditions that result in expression of the gRNA in at least one engineered cell; and screening the population of engineered cells to identify at least one engineered cell that expresses the functional chromogenic protein.

In some embodiments, the engineered cell or microbial organism comprises a fourth polynucleotide sequence that further comprises a first auxotrophic, antibiotic, or other selectable marker and a fifth polynucleotide sequence that further comprises a second auxotrophic, antibiotic, or other selectable marker, wherein the first auxotrophic, antibiotic, or other selectable marker and the second auxotrophic, antibiotic, or other selectable marker are different markers, and the method further comprises:

culturing the population of engineered cells or engineered microbial organisms in the presence of a counterselection agent or selection agent for the first auxotrophic, antibiotic, or other selectable marker; and

selecting for engineered cells or engineered microbial organisms that do not express the fourth polynucleotide sequence;

thereby preventing expression of the first gRNA and preventing alteration of the first phenotype coding sequence.

In some embodiments, the engineered cell or microbial organism comprises a fourth polynucleotide sequence that further comprises a first auxotrophic, antibiotic, or other selectable marker and a fifth polynucleotide sequence that further comprises a second auxotrophic, antibiotic, or other selectable marker, wherein the first auxotrophic, antibiotic, or other selectable marker and the second auxotrophic, antibiotic, or other selectable marker are different markers, and the method further comprises:

culturing the population of engineered cells or engineered microbial organisms in the presence of a counterselection agent or selection agent for the second auxotrophic, antibiotic, or other selectable marker; and

selecting for engineered cells or engineered microbial organisms that do not express the fifth polynucleotide sequence;

thereby preventing expression of the first gRNA and preventing alteration of the second phenotype coding sequence.

In some embodiments, (i) the first phenotype coding sequence encodes a functional first protein having a first detectable phenotype and the Cas9 nuclease cleaves the first phenotype coding sequence and disrupts expression of the first protein; and/or (ii) the second phenotype coding sequence encodes a functional second protein having a second detectable phenotype and the Cas9 nuclease cleaves the second phenotype coding sequence and disrupts expression of the second protein.

In some embodiments, (i) the first phenotype sequence comprises an engineered disruption in a sequence encoding a first protein that prevents expression of the first detectable phenotype, and the method comprises cleaving the first coding sequence at the engineered disruption with the Cas9 nuclease and repairing the first coding sequence to permit expression of the first protein; and/or (ii) the second phenotype sequence comprises an engineered disruption in a sequence encoding a second protein that prevents expression of the second detectable phenotype, and the method comprises cleaving the second coding sequence at the engineered disruption with the Cas9 nuclease and repairing the second coding sequence to permit expression of the second protein. In both cases, the engineered changes are defined by the sequence in the homologous repair donor sequence.

In some embodiments, (i) the first phenotype coding sequence encodes a functional first protein having a first detectable phenotype and the Cas9 nuclease cleaves the first phenotype coding sequence and disrupts expression of the first protein; and the second phenotype sequence comprises an engineered disruption in a sequence encoding a second protein that prevents expression of the second detectable phenotype, and the method comprises cleaving the second coding sequence at the engineered disruption with the Cas9 nuclease and repairing the second coding sequence to permit expression of the second protein; or (ii) the first phenotype sequence comprises an engineered disruption in a sequence encoding a first protein that prevents expression of the first detectable phenotype, and the method comprises cleaving the first coding sequence at the engineered disruption with the Cas9 nuclease and repairing the first coding sequence to permit expression of the first protein; and the second phenotype coding sequence encodes a functional second protein having a second detectable phenotype and the Cas9 nuclease cleaves the second phenotype coding sequence and disrupts expression of the second protein. In both cases, the engineered disruption is defined by the sequence in the homologous repair donor sequence.

In some embodiments, the engineered cell is a eukaryotic cell selected from the group consisting of yeast cell, mammalian cell, insect cell, or plant cell. In some embodiments, the engineered cell is a yeast cell and the cell comprises a polynucleotide sequence that comprises a polynucleotide encoding a Cas9 nuclease that is codon optimized for expression in yeast.

In some embodiments, the engineered cell is a prokaryotic cell, e.g., from a bacterium. In some embodiments, the engineered cell is a bacterial cell (e.g., E. coli) and the cell comprises a polynucleotide sequence that comprises a polynucleotide encoding a Cas9 nuclease that is codon optimized for expression in bacteria. In some embodiments, the cell comprises a polynucleotide sequence that comprises a polynucleotide encoding a S. pyogenes Cas9 nuclease.

In some embodiments, the engineered organism is a microbial organism, e.g., a bacterium. In some embodiments, the engineered organism is eukaryotic, e.g., a yeast, nematode, or plant.

In still another aspect, modular systems and methods for training an individual in laboratory procedures and the use of laboratory equipment for gene expression and gene editing are provided. In some embodiments, the modular system comprises:

(a) a module for expressing a detectable phenotype and/or for expressing a broken gene in a cell or organism, comprising an engineered cell or engineered microbial organism as disclosed herein and further comprising one or more reagents for culturing, transfecting, and/or transforming the cell or organism;

(b) a module for altering expression of the detectable phenotype and/or for repairing the broken gene in the cell or organism, comprising one or more reagents for expressing a Cas nuclease, expressing a gRNA, or preventing expression of a gRNA in the cell or organism;

(c) a module for analyzing the cell or organism, comprising one or more reagents for detecting the alteration of gene expression and/or repair of the broken gene in the cell or organism; and

(d) an instruction manual, comprising instructions for use of each of the modules.

In some embodiments, the instruction manual comprises digital materials, video, electronic media, electronic storage media, and/or optical media.

In some embodiments, the modular system further comprises an assessment module, comprising materials for evaluating a user's performance or skills in utilizing the modules (a), (b), and (c) and the instruction manual (d).

In some embodiments, the modular system comprises:

(a) a module for altering expression of an endogenous gene or genomic region in a cell or organism (e.g., disrupting or restoring the function of an endogenous gene or genomic region in the genome of the cell or organism), comprising an engineered cell or engineered microbial organism as disclosed herein, one or more reagents for expressing a Cas nuclease, and one or more reagents for targeting the endogenous gene or genomic region, and further comprising one or more reagents for culturing, transfecting, and/or transforming the cell or organism;

(b) a module for analyzing the cell or organism, comprising one or more reagents for detecting the alteration of gene expression (e.g., the disruption of function of the endogenous gene or genomic region or the restoration of function of the endogenous gene or genomic region); and

(c) an instruction manual, comprising instructions for use of each of the modules.

In some embodiments, the modular system further comprises an assessment module, comprising materials for evaluating a user's performance or skills in utilizing the modules (a) and (b) and the instruction manual (c).

### BRIEF DESCRIPTION OF THE DRAWINGS

FIG. 1. Schematic depicting an organism containing multiple color genes (A, B, and C) and the genes necessary for targeted knock-out of each of the color genes (gRNA A, B, and C, targeting color genes A, B, and C, respectively, and Cas9 nuclease).

FIG. 2. Yeast Version 1. The yeast strain contains a gene encoding chromogenic protein A under the control of a constitutive promoter, a gene encoding chromogenic protein B under the control of a constitutive promoter, a yeast optimized gene encoding Cas9 under the control of a galactose-inducible promoter, a plasmid comprising gRNA A for targeting chromogenic protein A and the auxotrophic marker URA3, and a plasmid comprising gRNA B for targeting chromogenic protein B and the auxotrophic marker LYS2.

FIG. 3. Yeast Version 2. The yeast strain contains a gene encoding chromogenic protein A under the control of a constitutive promoter, a gene encoding the ADE2 gene under the control of a native promoter, a yeast optimized gene encoding Cas9 under the control of a galactose-inducible promoter, a plasmid comprising gRNA A for targeting chromogenic protein A and the auxotrophic marker URA3, and a plasmid comprising gRNA B for targeting ADE2 and the auxotrophic marker LYS2.

FIG. 4. Yeast Version 3. The yeast strain contains a gene encoding chromogenic protein yEmRFP under the control of a constitutive promoter, a gene encoding the ADE2 gene under the control of a native promoter, a yeast optimized gene encoding Cas9 under the control of a galactose-inducible promoter, a plasmid comprising gRNA A for targeting yEmRFP and the auxotrophic marker URA3, and a plasmid comprising gRNA B for targeting ADE2 and the auxotrophic marker LYS2.

FIG. 5. Yeast Version 4. The yeast strain contains a gene encoding chromogenic protein yEmRFP under the control of a constitutive promoter, a gene encoding an ADE2 gene having an engineered disruption that causes a frameshift and prevents expression of ADE2 and under the control of a native promoter, a yeast optimized gene encoding Cas9 under the control of a galactose-inducible promoter, a plasmid comprising gRNA A for targeting yEmRFP and the auxotrophic marker URA3, and a plasmid comprising gRNA B for targeting the ADE2 having an engineered disruption and the auxotrophic marker LYS2.

FIG. 6. Bacteria Version 1. The bacteria express a chromogenic or fluorescent protein (GFP) under the control of a constitutive or inducible promoter. An episomal plasmid that expresses a gRNA to GFP and expresses Cas9 under the control of an inducible promoter can be transformed into the bacteria. Note that if both GFP and Cas9 expression rely on inducible promoters, they may not use the same method of induction. In the absence of an inducer for the Cas9 promoter, no gene editing events take place and the bacteria are the color resulting from expression of chromogenic protein (GFP) (in the presence of inducer, if an inducible promoter is used for GFP). In the presence of the Cas9 promoter-specific inducer, Cas9 expression is induced and the gRNA will be incorporated into Cas9, leading to targeting of GFP. Repair of the Cas9 cleavage is directed by the HR donor DNA contained in the transformed plasmid, allowing incorporation of specific mutations that prevent GFP expression, such that the bacteria no longer express GFP and the bacterial colonies appear white in color.

FIG. 7. Bacteria Version 2. The bacteria express a chromogenic or fluorescent protein (GFP) under the control of a constitutive or inducible promoter. An episomal plasmid that expresses a gRNA to GFP and expresses Cas9 under the control of an inducible promoter can be transformed into the bacteria. Note that if both GFP and Cas9 expression rely on inducible promoters, they may not use the same method of induction. In the absence of an inducer for the Cas9 promoter, no gene editing events take place and the bacteria are the color resulting from expression of chromogenic protein (GFP) (in the presence of an inducer, if an inducible promoter is used for GFP). In the presence of the Cas9 promoter-specific inducer, Cas9 expression is induced and the gRNA will be incorporated into Cas9, leading to targeting of GFP. Repair of the Cas9 cleavage is directed by the HR donor DNA contained in the transformed plasmid, allowing incorporation of specific mutations that convert GFP to BFP or YFP, such that the bacteria express BFP or YFP and the bacterial colonies appear blue or yellow in color, respectively.

FIG. 8. Bacteria Version 3. The bacteria express a chromogenic or fluorescent protein (GFP) under the control of a constitutive or inducible promoter. An episomal plasmid that expresses a gRNA to GFP and expresses Cas9 under the control of an inducible promoter can be transformed into the bacteria. Note that if both GFP and Cas9 expression rely on inducible promoters, they may not use the same method of induction. In the absence of an inducer for the Cas9 promoter, no gene editing events take place and the bacteria are the color resulting from expression of chromogenic protein (GFP) (in the presence of inducer, if an inducible promoter is used for GFP). In the presence of the Cas9 promoter-specific inducer, Cas9 expression is induced and the gRNA will be incorporated into Cas9, leading to targeting of GFP. Repair of the Cas9 cleavage is directed by the ssDNA HR donor co-transformed with the plasmid, allowing incorporation of specific mutations that prevent GFP expression, such that the bacteria no longer express GFP and the bacterial colonies appear white in color.

FIG. 9. Bacteria Version 4. The bacteria express a chromogenic or fluorescent protein (GFP) under the control of a constitutive or inducible promoter. An episomal plasmid that expresses a gRNA to GFP and expresses Cas9 under the control of an inducible promoter (such as an arabinose-inducible promoter or a rhamnose-inducible promoter) can be transformed into the bacteria. Note that if both GFP and Cas9 expression rely on inducible promoters, they may not use the same method of induction. In the absence of an inducer for the Cas9 promoter, no gene editing events take place and the bacteria are the color resulting from expression of chromogenic protein (GFP) (in the presence of inducer, if an inducible promoter is used for GFP). In the presence of the Cas9 promoter-specific inducer, Cas9 expression is induced and the gRNA will be incorporated into Cas9, leading to targeting of GFP. Repair of the Cas9 cleavage is directed by the ssDNA HR donor co-transformed with the plasmid, allowing incorporation of specific mutations that convert GFP to BFP or YFP, such that the bacteria express BFP or YFP and the bacterial colonies appear blue or yellow in color, respectively.

FIG. 10. Bacteria Version 5. The bacteria express a non-functional chromogenic or fluorescent protein (GFP) under the control of a constitutive or inducible promoter. An episomal plasmid that expresses a gRNA and expresses Cas9 under the control of an inducible promoter can be transformed into the bacteria. Note that if both GFP and Cas9 expression rely on inducible promoters, they may not use the same method of induction. In the absence of an inducer for the Cas9 promoter, no gene editing events take place and the bacteria are the color white, even in the presence of inducer, if an inducible promoter is used for GFP. In the presence of the Cas9 promoter-specific inducer, Cas9 expression is induced and the gRNA will be incorporated into Cas9, leading to targeting of GFP. Repair of the Cas9 cleavage is directed by the HR donor DNA contained in the transformed plasmid, allowing incorporation of specific mutations that restore GFP expression, such that the bacteria will express a functional GFP and the bacterial colonies appear green in color.

FIG. 11. Bacteria Version 6. The bacteria express a non-functional chromogenic or fluorescent protein (GFP) under the control of a constitutive or inducible promoter. An episomal plasmid that expresses a gRNA and expresses Cas9 under the control of an inducible promoter can be transformed into the bacteria. Note that if both GFP and Cas9 expression rely on inducible promoters, they may not use the same method of induction. In the absence of an inducer for the Cas9 promoter, no gene editing events take place and the bacteria are the color white, even in the presence of inducer, if an inducible promoter is used for GFP. In the presence of the Cas9 promoter-specific inducer, Cas9 expression is induced and the gRNA will be incorporated into Cas9, leading to targeting of GFP. Repair of the Cas9 cleavage is directed by the ssDNA HR donor co-transformed with the plasmid, allowing incorporation of specific mutations that restore GFP expression, such that the bacteria will express a functional GFP and the bacterial colonies appear green in color.

FIG. 12. Bacteria Version 7. The bacteria are genetically altered to have inducible Cas9 and inducible lambda red expression cassettes inserted within the bacterial genome. Both of these inducible promoters have loxP or loxP variant sequences that flank a transcription terminator sequence directly following the promoter, which block transcription and prevent expression of the induced proteins. The genetically altered bacteria are transformed with a single plasmid that contains an antibiotic resistance cassette (ARC) lacking its own promoter that is flanked on the 5′ and 3′ ends with short portions of an endogenous target gene (LacZ), an sgRNA that recognizes the targeted gene (“sgRNA-LZ”), a constitutively expressed Cre-recombinase cassette, and a “self-destruct” sgRNA that targets a self region within the plasmid, termed a non-repairable DSB site (“sgRNA-plasmid 1”). Upon expression of Cre recombinase, both of the transcription termination sequences will be excised, leaving a single loxP or loxP variant “scar” behind, and transcription and translation will become undeterred. A constitutively active Cas9 cassette will initiate Cas9 expression. The plasmid is destroyed; only bacteria that integrate the ARC will survive selection by that antibiotic. Upon induction of CRISPR-Cas and lambda red, recombination repair will allow the cell to express the antibiotic resistance protein and lose normal targeted gene expression (LacZ); these bacteria will survive in the presence of the antibiotic and will exhibit a loss of the normal targeted gene's function (e.g., loss of LacZ expression).

FIG. 13. Bacteria Version 8. Bacteria are genetically altered to have inducible Cas9 and Ku and LigD expression cassettes present. Additionally, an endogenous gene is replaced with an open reading frame (ORF) for a particular phenotype of interest (e.g., chromogenic protein ORF). These altered bacteria will express the chromogenic protein upon activation of the endogenous gene. The genetically altered bacteria are transformed with a single plasmid that has a cassette driving expression of an sgRNA that recognizes the chromogenic protein. Upon induction, a DSB occurs within the chromogenic protein. In contrast to unaltered E coli, the altered bacteria are able to perform non-homologous end joining (NHEJ)-based repair of the DSB break. Ku and LigD proteins will recognize the DSB and rejoin the two broken ends.

FIG. 14. Bacteria Version 9. Bacteria are genetically altered to have inducible CRISPR activity and inducible lambda red expression cassettes. Upon induction of CRISPR and lambda red recombination, an ORF (e.g., encoding an antibiotic resistance cassette (ARC)) lacking its own promoter that is flanked on the 5′ and 3′ ends with homology arms derived from an endogenous target gene (LacZ) will replace portions of the endogenous gene. This results in a loss of function for the endogenous gene, and instead the native or altered endogenous promoter drives the expression of the novel ORF, giving the bacteria a gain of function simultaneous to loss of function of the endogenous gene. Surviving bacteria will be screened for and categorized for ratios of colonies exhibiting the loss and the gain of phenotypes.

FIGS. 15A-15C. Cas9-mediated integration of chloramphenicol (CAM) resistance in bacteria. (A) Number of CAMP colonies electroporated with either gRNA/donorDNA or water (control). (B) PCR schema for detection of wild-type LacZ and Cas9-directed integration of CAM-resistance cassette into LacZ. (C) PCR results from 13 colonies screened. Arrows indicate successful integration of CAM-resistance cassette.

FIGS. 16A-16D. Blue/white screening to detect CRISPR and lambda red dependent stop codon inserted into LacZ reading frame. (A-C) Representative bacterial plates used for blue/white screening. Bacteria were transformed with plasmids containing the following expression cassettes: a Cas9 cassette but no gRNA expressing cassette (A, Control Transformation 1), a gRNA expressing cassette but no Cas9 expressing cassette (B, Control Transformation 2), or a gRNA expressing cassette and a Cas9 expressing cassette (C, CRISPR Transformation.) Without CRISPR activity, 100% of cells were blue showing normal β-galactosidase activity. When CRISPR was activated, only 43 of the ˜350 colonies seen were blue while approximately 88% of 350 colonies were white displaying a lack of β-galactosidase activity. (D) Table of results.

FIG. 17A-17D. PCR screening and sequencing to detect CRISPR and lambda red dependent stop codon inserted into LacZ reading frame. (A) PCR genotyping strategy. Wild-type and mutant E. coli (FWT1 and FWT2) produced a 686 base pair amplicon. LacZ mutant E. coli (FWT1 and Rmut1) produce a 550 base pair amplicon. (B) DNA gel image from genotyping PCR. Lanes 1-20 are from control transformation 2. Lanes 21-39 are from CRISPR transformation. Wild-type band is white arrow. Mutant only band is black arrow. (C) Table of results from PCR genotyping. (D) Representative Sanger sequencing with wild-type LacZ locus sequence and traces from wild-type E. coli and premature stop codon inserted E. coli.

### DETAILED DESCRIPTION OF THE INVENTION

### I. INTRODUCTION

Provided herein are compositions, kits, and methods for using the CRISPR/Cas system for altering gene expression in cells and/or organisms, such as microbial organisms. As described herein, in some embodiments, the control of gene expression in a cell or an organism (e.g., a microbe, e.g., yeast) due to CRISPR/Cas activity can be visualized using polynucleotides comprising coding sequences for proteins that produce a detectable phenotype in the cell or organism, e.g., chromogenic and/or fluorogenic proteins, proteins that regulate odor, proteins that regulate morphology, resistance to an antibiotic, or enzymes that produce a detectable enzymatic product. In some embodiments, control of gene expression in the cell or organism is visualized using polynucleotides comprising coding sequences for chromogenic and/or fluorogenic proteins. In some embodiments, endogenous genes or genomic targets are targeted for alteration using the CRISPR/Cas system. Through the use of an inducible promoter to control expression of the CRISPR/Cas system, changes in phenotype (e.g., color) in the cell or organism due to gene disruption and repair can be readily detected. In one aspect, the disclosure provides compositions, kits, and methods for using the CRISPR/Cas system with or without lambda red recombineering.

Also provided herein are compositions, kits, and methods for using lambda red recombineering, with or without the CRISPR/Cas system, for altering gene expression in cells and/or organisms, such as microbial organisms. As described herein, lambda red can be used on its own to target and replace regions within genomic DNA, although lambda red recombineering is less efficient without the directed nuclease activity from Cas9-sgRNA functional units. Thus, the success rate of lambda red recombineering is quantifiably higher when it is used in conjunction with CRISPR/Cas. Accordingly, in one aspect, the compositions, kits, and methods provided herein can be used for comparative analyses of gene targeting when the CRISPR/Cas system is used versus in its absence.

### II. DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document.

As used herein, the term “Cas nuclease” or “Cas” refers to CRISPR associated protein, an RNA-guided nuclease that introduces a double stranded break in nucleic acid. In some embodiments, the Cas nuclease is CRISPR associated protein 9 (“Cas9 nuclease” or “Cas9”).

The term “guide RNA” or “gRNA,” as used herein, refers to a nucleic acid sequence that guides a nuclease (e.g., Cas nuclease) to a target nucleic acid site to be cleaved. Typically, a gRNA comprises a “scaffold” sequence for binding the nuclease and a “targeting” sequence that defines the target nucleic acid site (e.g., genomic DNA site) to be cleaved. In some embodiments, the gRNA comprises a targeting sequence that has a length of about 20 nucleotides.

The terms “lambda red” or “lambda red system,” as used herein, refers to a lambda red recombineering system that is derived from the lambda red bacteriophage. The lambda red recombineering system has three components: lambda exonuclease (“Exo”), beta protein (“Beta”), and gamma protein (“Gam”). In some embodiments, “a polynucleotide encoding lambda red” refers to a polynucleotide that encodes the Exo, Beta, and Gam components of the lambda red system.

As used herein, the terms “nucleic acid” and “polynucleotide” interchangeably refer to DNA, RNA, and polymers thereof in single-stranded, double-stranded, or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

Sequences are “substantially identical” to each other if they have a specified percentage of nucleotides that are the same (e.g., at least 60%, 65%, 70%, 75%, 80%, 85%, 90%, 91%, 92%, 93%, 94%, 95%, 96%, 97%, 98%, or 99% identity over a specified region), when compared and aligned for maximum correspondence over a comparison window, or designated region as measured using one of the following sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Accelrys), or by manual alignment and visual inspection.

Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “promoter” refers to regions or sequence located upstream and/or downstream from the start of transcription and which are involved in recognition and binding of RNA polymerase and other proteins to initiate transcription.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a different gene in the same species).

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “expression cassette” refers to a nucleic acid construct that, when introduced into a host cell, results in transcription and/or translation of an RNA or polypeptide, respectively.

A “vector” refers to a polynucleotide, which when independent of the host chromosome, is capable replication in a host organism. Preferred vectors include plasmids and typically have an origin of replication. Vectors can comprise, e.g., transcription and translation terminators, transcription and translation initiation sequences, and promoters useful for regulation of the expression of the particular nucleic acid.

### III. ENGINEERED CELLS AND ORGANISMS

In one aspect, engineered organisms and engineered cells are provided that comprise one or more phenotype coding sequences, a Cas nuclease, and a guide RNA (gRNA) that corresponds to the phenotype coding sequence. In some embodiments, the organisms and engineered cells comprise two or more phenotype coding sequences and further comprise a separate gRNA that corresponds to each of the two or more phenotype coding sequences. The terms “first,” “second,” “third,” “fourth,” and “fifth,” when used with reference to polynucleotide sequences, promoters, and auxotrophic, antibiotic, or other selectable markers, is simply to more clearly distinguish the polynucleotide sequences, promoters, and auxotrophic, antibiotic, or other selectable markers, and is not intended to indicate order.

In some embodiments, the engineered organism (e.g., engineered microbial organism or engineered eukaryotic organism) or the engineered cell (e.g., an engineered microbial cell or an engineered eukaryotic cell) comprises:

an endogenous gene or genomic region to be targeted for gene alteration, wherein the endogenous gene or genomic region is a functional endogenous gene or genomic region having a detectable phenotype or comprises a disruption that prevents expression of the detectable phenotype; or one or more heterologous polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype;

a heterologous polynucleotide sequence comprising a promoter operably linked to a polynucleotide encoding a Cas nuclease; and

a heterologous polynucleotide sequence comprising a guide RNA (gRNA) that targets the endogenous gene, genomic region, or phenotype coding sequence.

In some embodiments, the engineered organism or the engineered cell comprises:

an endogenous gene or genomic region to be targeted for gene alteration, wherein the endogenous gene or genomic region is a functional endogenous gene or genomic region having a detectable phenotype or comprises a disruption that prevents expression of the detectable phenotype; or a heterologous polynucleotide sequence comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype;

a heterologous polynucleotide sequence comprising a promoter operably linked to a polynucleotide encoding lambda red; and

a heterologous polynucleotide sequence comprising a homologous donor DNA sequence.

In some embodiments, the engineered organism or the engineered cell comprises:

an endogenous gene or genomic region to be targeted for gene alteration, wherein the endogenous gene or genomic region is a functional endogenous gene or genomic region having a detectable phenotype or comprises a disruption that prevents expression of the detectable phenotype; or a heterologous polynucleotide sequence comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype;

a heterologous polynucleotide sequence comprising a promoter operably linked to a polynucleotide encoding a Cas nuclease (e.g., Cas9 nuclease);

a heterologous polynucleotide sequence comprising a promoter operably linked to a polynucleotide encoding lambda red;

a heterologous polynucleotide sequence comprising a gRNA that targets the endogenous gene, genomic region, or chromogenic coding sequence; and

a heterologous polynucleotide sequence comprising a homologous donor DNA sequence.

In some embodiments, wherein the engineered cell comprises both a polynucleotide encoding a Cas nuclease and a polynucleotide encoding lambda red, the Cas nuclease polynucleotide sequence and the lambda red polynucleotide sequence are operably linked.

In some embodiments, the engineered organism or the engineered cell comprises:

a heterologous polynucleotide sequence comprising a chromogenic coding sequence that encodes a functional chromogenic protein;

a heterologous polynucleotide sequence comprising a promoter operably linked to a polynucleotide encoding a Cas nuclease (e.g., Cas9 nuclease);

a heterologous polynucleotide sequence comprising a gRNA that targets the endogenous gene, genomic region, or chromogenic coding sequence; and

a heterologous polynucleotide sequence comprising a homologous donor DNA sequence.

In some embodiments, the phenotype is a chromogenic phenotype, e.g., a color or fluorescence. In some embodiments, the chromogenic coding sequence encodes a non-fluorescent color protein. In some embodiments, the chromogenic coding sequence encodes a fluorogenic protein, e.g., a green fluorescent protein (GFP), blue fluorescent protein (BFP), red fluorescent protein (RFP), orange fluorescent protein (OFP), yellow fluorescent protein (YFP), cyan fluorescent protein (CFP), coral fluorescent protein, and derivatives or variants thereof. In some embodiments, the chromogenic coding sequence encodes a fluorogenic protein and the homologous donor DNA comprises a mutation that prevents expression of the fluorogenic protein. In some embodiments, the chromogenic coding sequence encodes a fluorogenic protein and the homologous donor DNA comprises a mutation that modifies expression of the fluorogenic protein from a first color to a second color (e.g., from GFP to BFP or YFP). In some embodiments, the chromogenic coding sequence comprises an engineered disruption that prevents expression of the detectable protein (e.g., a “broken” fluorogenic protein) and the homologous donor DNA comprises a mutation that restores expression of the fluorogenic protein.

In some embodiments, the engineered organism (e.g., engineered microbial organism or engineered eukaryotic organism) or the engineered cell (e.g., an engineered microbial cell or an engineered eukaryotic cell) comprises:

a first heterologous polynucleotide sequence comprising a first phenotype coding sequence operably linked to a first promoter, wherein the first phenotype coding sequence either (i) encodes a functional first protein having a first detectable phenotype or (ii) comprises an engineered disruption in a sequence encoding a first protein that prevents expression of the first detectable phenotype;

a second heterologous polynucleotide sequence comprising a second phenotype coding sequence operably linked to a second promoter, wherein the second phenotype coding sequence either (i) encodes a functional second protein having a second detectable phenotype or (ii) comprises an engineered disruption in a sequence encoding a second protein that prevents expression of the second detectable phenotype;

a third heterologous polynucleotide sequence comprising a third promoter operably linked to a polynucleotide encoding a Cas nuclease (e.g., Cas9 nuclease);

a fourth heterologous polynucleotide sequence comprising a first gRNA that targets the first chromogenic coding sequence; and

a fifth heterologous polynucleotide sequence comprising a second gRNA that targets the second chromogenic coding sequence.

In some embodiments, the detectable phenotype or each of the one or more detectable phenotypes (e.g., each of the first detectable phenotype and the second detectable phenotype) is a detectable color, fluorescence, scent, enzymatic activity, antibiotic resistance (gain or loss), morphology, or lethality. In some embodiments, both the first detectable phenotype and the second detectable phenotype are the same category of phenotype (e.g., both are detectable colors and/or fluorescences; both are detectable scents; both are detectable enzymatic activities; both are antibiotic resistance (gain or loss); or both are detectable morphologies). In some embodiments, the first detectable phenotype is a different category of phenotype than the second detectable phenotype (e.g., one is a detectable color and/or fluorescence and one is a detectable scent).

In some embodiments, the first detectable phenotype and the second detectable phenotype is a detectable color and/or fluorescence, and the engineered organism (e.g., engineered microbial organism or engineered eukaryotic organism) or the engineered cell (e.g., an engineered microbial cell or an engineered eukaryotic cell) comprises:

a first heterologous polynucleotide sequence comprising a first chromogenic coding sequence operably linked to a first promoter, wherein the first chromogenic coding sequence either (i) encodes a functional first chromogenic protein or (ii) comprises an engineered disruption in a sequence encoding a first chromogenic protein that prevents expression of the first chromogenic protein;

a second heterologous polynucleotide sequence comprising a second chromogenic coding sequence operably linked to a second promoter, wherein the second chromogenic coding sequence either (i) encodes a functional second chromogenic protein or (ii) comprises an engineered disruption in a sequence encoding a second chromogenic protein that prevents expression of the second chromogenic protein;

a third heterologous polynucleotide sequence comprising a third promoter operably linked to a polynucleotide encoding a Cas9 nuclease;

a fourth heterologous polynucleotide sequence comprising a first guide RNA (gRNA) that targets the first chromogenic coding sequence; and

a fifth heterologous polynucleotide sequence comprising a second gRNA that targets the second chromogenic coding sequence.

In some embodiments, the engineered organism is a microbial organism. In some embodiments, the engineered organism is a eukaryotic organism. In some embodiments, the engineered organism is an engineered plant.

In some embodiments, the engineered cell is a cell obtained from a microbial organism, prokaryotic organism, or eukaryotic organism as described herein. In some embodiments, the engineered cell is a microbial cell, e.g., a cell from a bacterium, phytoplasma, virus, viroid, protozoan, rickettsia, or fungus. In some embodiments, the engineered cell is a prokaryotic cell, e.g., from a bacterium. In some embodiments, the engineered cell is eukaryotic cell, e.g., a yeast cell, a plant cell, an insect cell, or a mammalian cell.

### Cells and Organisms

In some embodiments, the engineered cell is a cell from a microbial organism, e.g., a bacterium, phytoplasma, virus, viroid, protozoan, rickettsia, or fungus. In some embodiments, the engineered cell is a eukaryotic cell. In some embodiments, the engineered cell is a prokaryotic.

In some embodiments, the engineered cell is fungal, e.g., from a species of yeast, mold, or filamentous fungus. In some embodiments, the cell is a eukaryotic cell from a filamental fungus, e.g., a species of Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, or Volvariella. In some embodiments, the cell is a eukaryotic cell from a yeast, e.g., a species of Candida, Hansenula, Saccharomyces, Schizosaccharomyces, Pichia, Kluyveromyces, or Yarrowia. In some embodiments, the cell is a eukaryotic cell from a species of Saccharomyces, e.g., S. cerevisiae. In some embodiments, the cell is a eukaryotic cell from a species of Schizosaccharomyces, e.g., S. pombe.

In some embodiments, the cell is a prokaryotic cell from a bacterium, e.g., a species of Escherichia, Streptomyces, Zymonas, Acetobacter, Citrobacter, Synechocystis, Rhizobium, Clostridium, Corynebacterium, Streptococcus, Xanthomonas, Lactobacillus, Lactococcus, Bacillus, Alcaligenes, Pseudomonas, Aeromonas, Azotobacter, Comamonas, Mycobacterium, Rhodococcus, Gluconobacter, Ralstonia, Acidithiobacillus, Microlunatus, Geobacter, Geobacillus, Arthrobacter, Flavobacterium, Serratia, Saccharopolyspora, Thermus, Stenotrophomonas, Chromobacterium, Sinorhizobium, Saccharopolyspora, Agrobacterium, Paracoccus, or Pantoea. In some embodiments, the cell is a prokaryotic cell from a species of Escherichia, e.g., E. coli.

In some embodiments, the cell is a protist cell, e.g., a species of Dictyostelium.

In some embodiments, the cell is a eukaryotic cell from a plant, e.g., a cell from Nicotiana, Arabidopsis, flowering plant, or crop-bearing plant (e.g., a fruit or vegetable plant). In some embodiments, the cell is a eukaryotic cell from a nematode, e.g., from C. elegans. In some embodiments, the cell is a eukaryotic cell from an insect, e.g., a cell line such as S2, Sf9, or Sf21. In some embodiments, the cell is a eukaryotic cell from a mammal, e.g., mouse, rat, human, Chinese hamster, or canine primary or cell lines, including but not limited to HeLa, CHO, and MDCK.

In some embodiments, the engineered organism is a microbial organism, e.g., a bacterium, phytoplasma, virus, viroid, protozoan, rickettsia, or fungus. In some embodiments, the engineered organism is eukaryotic. In some embodiments, the engineered organism is prokaryotic.

In some embodiments, the engineered organism is fungal, i.e., a eukaryotic organism within the kingdom of fungi. Fungi may include yeasts, molds, and filamentous fungi. In some embodiments, the eukaryotic organism is a filamental fungus, e.g., a species of Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, or Volvariella. In some embodiments, the eukaryotic organism is a yeast, e.g., a species of Candida, Hansenula, Saccharomyces, Schizosaccharomyces, Pichia, Kluyveromyces, or Yarrowia. In some embodiments, the eukaryotic organism is a species of Saccharomyces, e.g., S. cerevisiae. In some embodiments, the eukaryotic organism is a species of Schizosaccharomyces, e.g., S. pombe.

In some embodiments, the engineered organism is bacterial, e.g., a species of Escherichia, Streptomyces, Zymonas, Acetobacter, Citrobacter, Synechocystis, Rhizobium, Clostridium, Corynebacterium, Streptococcus, Xanthomonas, Lactobacillus, Lactococcus, Bacillus, Alcaligenes, Pseudomonas, Aeromonas, Azotobacter, Comamonas, Mycobacterium, Rhodococcus, Gluconobacter, Ralstonia, Acidithiobacillus, Microlunatus, Geobacter, Geobacillus, Arthrobacter, Flavobacterium, Serratia, Saccharopolyspora, Therms, Stenotrophomonas, Chromobacterium, Sinorhizobium, Saccharopolyspora, Agrobacterium, Paracoccus, or Pantoea. In some embodiments, the engineered organism is a species of Escherichia, e.g., E. coli.

In some embodiments, the engineered organism is a protist, e.g., a species of Dictyostelium.

In some embodiments, the engineered organism is a nematode, e.g., C. elegans.

In some embodiments, the engineered organism is a plant, e.g., a monocot or dicot plant. In some embodiments, the plant is Nicotiana, Arabidopsis, a flowering plant, or a crop-bearing plant (e.g., a fruit or vegetable plant).

### Phenotype Coding Sequences

In one aspect, the engineered organisms and engineered cells disclosed herein comprise one or more phenotype coding sequences. In some embodiments, the engineered organism or engineered cell comprises one phenotype coding sequence. In some embodiments, the engineered organism or engineered cell comprises multiple phenotype coding sequences, e.g., 2, 3, 4, 5, 6, or more phenotype coding sequences. In some embodiments, the phenotype coding sequences are chromogenic coding sequences. As used herein, the term “chromogenic” encompasses both color and fluorescence as the detectable phenotype. In some embodiments, the phenotype coding sequences are scent coding sequences. In some embodiments, the phenotype coding sequences are enzymatic activity coding sequences. In some embodiments, the phenotype coding sequences are morphology coding sequences. In some embodiments, the phenotype coding sequences are lethality coding sequences. In some embodiments, the phenotype coding sequences are sequences that code for the gain or loss of resistance to an antibiotic. For example, in some embodiments, the antibiotic resistance phenotype coding sequences can be used for replacing one antibiotic resistance for another in the organism or cell (i.e., simultaneous gain and loss of function).

In some embodiments, the engineered organism or engineered cell comprises multiple chromogenic coding sequences, e.g., 2, 3, 4, 5, 6, or more chromogenic coding sequences. In some embodiments, a chromogenic coding sequence encodes a fluorogenic protein, e.g., a green fluorescent protein (GFP), blue fluorescent protein (BFP), red fluorescent protein (RFP), orange fluorescent protein (OFP), yellow fluorescent protein (YFP), cyan fluorescent protein (CFP), coral fluorescent proteins, luciferase, and derivatives or variants thereof. In some embodiments, a chromogenic coding sequence encodes a coral fluorescent protein. In some embodiments, a chromogenic coding sequence encodes a chromogenic (non-fluorogenic) protein. Chromogenic coding sequences are commercially available, e.g., ProteinPaintbox® fluorescent and chromogenic proteins (ATUM, Newark, Calif.).

In some embodiments, the one or more phenotype coding sequences encodes for one or more components of a metabolic pathway where providing a substrate for the enzymatic reaction results in the production of a smell. As a non-limiting example, the alcohol acetyltransferase gene, ATF1, can be introduced into a cell (e.g., bacterial cell) and in the presence of isoamyl alcohol, the cell metabolizes the isoamyl alcohol to emit the smell of bananas.

In some embodiments, the one or more phenotype coding sequences encodes for gain or loss of resistance to an antibiotic. As a non-limiting example, a first phenotype coding sequence encoding for resistance to a first antibiotic (e.g., Kanamyin) can be disrupted or replaced with a second phenotype coding sequence encoding for resistance to a second antibiotic (e.g., Spectinomycin).

In some embodiments, one or more phenotype coding sequences is optimized or enhanced for expression in the organism or cell in which the coding sequence is to be expressed, e.g., a cell or organism as described herein. In some embodiments, the chromogenic coding sequence is optimized or enhanced for expression in the organism, e.g., the engineered organism is a yeast and the chromogenic coding sequence is yeast-enhanced. Enhanced or optimized chromogenic sequences are described in the art. See, e.g., Keppler-Ross et al., Genetics, 2008, 179:705-710.

In some embodiments, one or more of the phenotype coding sequences (e.g., chromogenic coding sequences) is in-frame and encodes a functional protein (e.g., a functional chromogenic protein). In some embodiments, one or more of the phenotype coding sequences (e.g., chromogenic coding sequences) comprises an engineered disruption in the polynucleotide sequence that prevents expression of the protein having the detectable phenotype (e.g., chromogenic protein). In some embodiments, the engineered disruption is an insertion of a whole open reading frame (ORF) into an existing ORF. In some embodiments, the engineered disruption is an insertion of about 1 to about 20 nucleotides relative to the sequence that encodes the functional protein, e.g., about 1-20, 1-15, 1-10, 2-20, 2-15, 3-20, 3-10, 4-20, 4-10, 5-20, or 5-10 nucleotides relative to the sequence that encodes the functional protein. In some embodiments, the engineered disruption is an insertion of no more than about 20 nucleotides relative to the sequence that encodes the functional protein, e.g., no more than about 15 nucleotides, no more than about 10 nucleotides, or no more than about 5 nucleotides relative to the sequence that encodes the functional protein. In some embodiments, the engineered organism or engineered cell comprises at least a first phenotype coding sequence (e.g., chromogenic coding sequence) that encodes a functional first protein (e.g., first chromogenic protein) and further comprises at least a second phenotype coding sequence (e.g., chromogenic coding sequence) that comprises an engineered disruption in the polynucleotide sequence that prevents expression of the second protein (e.g., second chromogenic protein).

In some embodiments, the engineered organism or engineered cell comprises one or more phenotype coding sequences (e.g., chromogenic coding sequences) and further comprises a coding sequence for a metabolic pathway gene that, depending on whether the gene is expressed or gene expression is prevented, results in a change in color in the organism. As a non-limiting example, in some embodiments, the engineered organism or cell can express a coding sequence for a gene within the adenine biosynthetic pathway (e.g., ADE2 or ADE5) or for a gene within the carotenoid synthesis pathway (e.g., phytoene synthase or lycopene beta cyclase). In some embodiments, the coding sequence for the adenine biosynthetic pathway gene (e.g., ADE2 or ADE5) or the carotenoid synthesis pathway contains an engineered disruption in the sequence that prevents expression of the protein, which in turn causes the organism to have a pink or red color.

In some embodiments, the engineered organism or engineered cell comprises one or more phenotype coding sequences (e.g., chromogenic coding sequences) and further comprises a coding sequence for a metabolic pathway gene that, depending on whether the gene is expressed or gene expression is prevented, results in a nutrient requirement. As a non-limiting example, in some embodiments, the engineered organism or cell can express a coding sequence for a gene within the leucine biosynthetic pathway (e.g. leuB). In some embodiments, the coding sequence for the leucine biosynthetic pathway gene (e.g., leuB) contains an engineered disruption in the sequence that prevents expression of the protein, which in turn causes the organism to have a leucine nutrient requirement.

In some embodiments, each of the phenotype coding sequences (e.g., chromogenic coding sequences) and/or coding sequences for a metabolic pathway gene that alters color in the organism (e.g., ADE2) is operably linked to a promoter. In some embodiments, the coding sequence for the metabolic pathway gene that alters color in the organism (e.g., ADE2) is a sequence containing an engineered disruption that prevents expression of the metabolic pathway gene, and is operably linked to its native promoter. In some embodiments, a phenotype coding sequence (e.g., chromogenic coding sequence is operably linked to a constitutive (constitutively active) promoter or an inducible promoter. In some embodiments, the promoter is a constitutively active promoter. Suitable constitutively active promoters include, but are not limited to, Tyr tRNA, Sigma 70 consensus sequence, TEF1, RPL18B, RNR2, TDH3, REV1, PGK, and ADH1.

In some embodiments, each polynucleotide comprising a phenotype coding sequence (e.g., chromogenic coding sequence) as described herein (e.g., a polynucleotide comprising a chromogenic coding sequence that is operably linked to an inducible promoter or a constitutively active promoter) is in a separate expression cassette, expression vector, or plasmid than the other polynucleotide sequences disclosed herein (e.g., polynucleotides comprising other phenotype coding sequences, polynucleotides encoding a Cas nuclease, or polynucleotides encoding a guide RNA). In some embodiments, a first polynucleotide comprising a first phenotype coding sequence as described herein is in the same expression cassette, vector, or plasmid as a second polynucleotide comprising a second phenotype coding sequence as described herein. In some embodiments, two, three, four, or more polynucleotides comprising two, three, four or more phenotype sequences are in the same expression cassette, vector, or plasmid. In some embodiments, one or more polynucleotides comprising one or more phenotype coding sequences as described herein are stably integrated into the genomic material (e.g., chromosome) of a cell or organism as described herein.

In some embodiments, an expression vector or plasmid, engineered cell, or engineered organism as described herein comprises multiple identical copies (e.g., at least 2, 3, 4, 5, 10, 20, 30, 40, 50, 60, 70, 80, 90, 100 or more copies) of a phenotype coding sequence. In some embodiments, an expression vector or plasmid, engineered cell, or engineered organism as described herein comprises multiple identical copies (e.g., at least 2, 3, 4, 5, 10, 20, 30, 40, 50, 60, 70, 80, 90, 100 or more copies) of a first phenotype coding sequence and multiple identical copies (e.g., at least 2, 3, 4, 5, 10, 20, 30, 40, 50, 60, 70, 80, 90, 100 or more copies) of a second phenotype coding sequence.

### Cas Nucleases

In some embodiments, a Cas9 nuclease is used to create a double-stranded break (DSB) in one or more phenotype coding sequences (e.g., chromogenic coding sequences and/or coding sequences for a metabolic pathway gene) as described herein. In some embodiments, the Cas nuclease is a Cas9 nuclease. The CRISPR/Cas system and Cas activity are described, e.g., in Jinek et al., Science, 2012, 337:816-821; and in Jinek et al., eLife, 2013, 2:e00471. Briefly, Cas cleaves DNA to generate blunt DSBs at sites defined by a guide RNA sequence. The DSBs can then be repaired in the cell or organism by homology-directed repair (HDR), nonhomologous end-joining (NHEJ), or alternative end-joining (A-EJ). In some embodiments, the coding sequence that is targeted by Cas (e.g., Cas9) encodes a functional chromogenic protein or functional metabolic pathway protein, and the cleavage and repair of the chromogenic coding sequence or coding sequence for a metabolic pathway gene disrupts expression of the chromogenic protein or metabolic pathway protein. In some embodiments, the coding sequence that is targeted by Cas (e.g., Cas9) comprises an engineered disruption in the sequence that prevents expression of a functional chromogenic protein or functional metabolic pathway protein, and the cleavage and repair of the chromogenic coding sequence or coding sequence for a metabolic pathway gene results in the expression of a functional chromogenic protein or metabolic pathway protein. In some embodiments, the coding sequence that is targeted by Cas9 encodes a functional chromogenic protein or functional metabolic pathway protein, and the cleavage and repair of the chromogenic coding sequence or coding sequence for a metabolic pathway gene changes expression of the chromogenic protein to a different chromogene or metabolic pathway protein to a different metabolic pathway.

Cas nucleases, such as Cas9 nucleases, can be derived from any of a variety of bacterial species, including but not limited to Streptococcus pyogenes, Streptococcus thermophilus, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Helicobacter mustelae, Bacillus cereus, Acidovorax ebreus, Pasteurella multocida subsp. Multocida, Sutterella wadsworthensis, proteobacterium, Parasutterella excrementihominis, Wolinella succinogenes, and Francisella novicida.

Cas9 polypeptide sequences, and polynucleotide sequences encoding Cas polypeptides, are known in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. In some embodiments, the Cas9 nuclease is a variant, e.g., a variant that has enhanced specificity (e.g., Cas9-nickase or dCas9-Fok1). Cas9 variants and methods of engineering and screening Cas9 variants are described in the art. See, e.g., Murovec et al., Plant Biotechnology Journal, 2017, 15:917-926; Sadhu et al., bioRxiv, 2017, doi: https://doi.org/10.1101/147637; and Casini et al., Nature Biotechnology, 2018, 36:265-271.

In some embodiments, the Cas9 polynucleotide sequence is codon-optimized for expression in a particular cell or organism. For example, in some embodiments, the Cas9 polynucleotide sequence is codon-optimized for expression in human cells. See, DiCarlo et al., Nucleic Acids Res, 2013, 41:4336-4343. In some embodiments, the Cas9 polynucleotide sequence is codon-optimized for expression in a microbial cell or organism as described herein, e.g., for expression in yeast or bacteria. In some embodiments, a yeast codon-optimized Cas9 polynucleotide sequence is provided as SEQ ID NO:1. In some embodiments, an E. coli codon optimized Cas9 polynucleotide is provided as SEQ ID NO:2. In some embodiments, a S. pyogenes Cas9 nuclease is provided as SEQ ID NO:3.

In some embodiments, the Cas nuclease is a variant or derivative of Cas9. For example, in some embodiments, the Cas nuclease is a nuclease that is substantially identical (e.g., at least 70, 75, 80, 85, 90, or 95% identical) to a Cas9 nuclease as disclosed herein. In some embodiments, the Cas nuclease is a nuclease that is substantially identical (e.g., at least 70, 75, 80, 85, 90, or 95% identical) to a S. pyogenes Cas9 nuclease, e.g., the S. pyogenes Cas9 nuclease set forth in NBCI Ref. Seq. No. NP_269215.

In some embodiments, the Cas nuclease is a destabilized variant of Cas9. Destabilized Cas9 nucleases are known in the art. See, e.g., Senturk et al., Nat Commun, 2017, 8:14370.

In some embodiments, the Cas nuclease is a Cas-12a nuclease (also known as Cpf1). Cas-12a nucleases are known in the art. See, e.g., Yan et al., Appl Environ Microbiol, 2017, 83(17); doi: 10.1128/AEM.00947-17. In some embodiments, the Cas nuclease is a Cas-like nuclease.

In some embodiments, a polynucleotide encoding a Cas9 nuclease is operably linked to an inducible promoter. In some embodiments, an inducible promoter is a promoter that can be turned on and off by adding or removing an inducing agent. In some embodiments, an inducing agent is a molecule such as doxycycline, tetracycline, galactose, arabinose, rhamnose, metal ions, alcohol, or a steroid compound. In some embodiments, an inducible promoter is a promoter that is activated by environmental conditions, for example, light or temperature. In some embodiments, the promoter is a galactose inducible promoter. In some embodiments, the promoter is a arabinose inducible promoter. In some embodiments, the promoter is a rhamnose-inducible promoter. In some embodiments, the promoter is a doxycycline inducible promoter. Inducible promoters are described in the art. See, e.g., Mumberg et al., Nucleic Acids Res., 1994, 22:5767-5768; Cao et al., Nucleic Acids Res, 2016, 44:e149. In some embodiments, a polynucleotide encoding a Cas9 nuclease is operably linked to a constitutively active promoter, e.g., a constitutively active promoter as described herein.

In some embodiments, an expression cassette, expression vector, or plasmid comprising a yeast or bacteria codon-optimized Cas9 nuclease polynucleotide sequence is provided. In some embodiments, the yeast codon-optimized Cas9 nuclease polynucleotide sequence has the sequence of SEQ ID NO:1 or is substantially identical to SEQ ID NO:1 (e.g., has at least 70%, 75%, 80%, 85%, 90%, 91%, 92%, 93%, 94%, 95%, 96%, 97%, 98%, or 99% identity to SEQ ID NO:1). In some embodiments, the E. coli codon-optimized Cas9 nuclease polynucleotide sequence has the sequence of SEQ ID NO:2 or is substantially identical to SEQ ID NO:2 (e.g., has at least 70%, 75%, 80%, 85%, 90%, 91%, 92%, 93%, 94%, 95%, 96%, 97%, 98%, or 99% identity to SEQ ID NO:2). In some embodiments, an expression cassette, expression vector, or plasmid comprising a S. pyogenes Cas9 nuclease polynucleotide sequence is provided. In some embodiments, the S. pyogenes Cas9 nuclease polynucleotide sequence has the sequence of SEQ ID NO:3 or is substantially identical to SEQ ID NO:3 (e.g., has at least 70%, 75%, 80%, 85%, 90%, 91%, 92%, 93%, 94%, 95%, 96%, 97%, 98%, or 99% identity to SEQ ID NO:3). In some embodiments, the Cas9 nuclease polynucleotide sequence further comprises a tag (e.g., a FLAG tag, an HA tag, a His tag, or a GST tag). In some embodiments, the expression cassette, expression vector, or plasmid comprising a yeast codon-optimized Cas9 nuclease polynucleotide sequence further comprises an inducible promoter as described herein (e.g., a galactose inducible promoter).

In some embodiments, the polynucleotide sequence comprising a polynucleotide encoding a Cas nuclease further comprises a sequence for a selectable marker, such as but not limited to an auxotrophic marker, an antibiotic resistance marker, or other selectable marker. In some embodiments, the selectable marker is suitable for selection of bacterial cells.

In some embodiments, the polynucleotide sequence comprising a polynucleotide encoding a Cas nuclease further comprises a sequence for an auxotrophic marker. In some embodiments, the auxotrophic marker is a yeast auxotrophic marker. Examples of auxotrophic markers include, but are not limited to, LEU2, URA3, LYS2 HIS3, MET17, and TRP1.

In some embodiments, the polynucleotide sequence comprising a polynucleotide encoding a Cas nuclease further comprises a sequence for an antibiotic resistance marker. Examples of antibiotic resistance markers include, but are not limited to, Ampicillin, Chloramphenicol, Kanamycin, Streptomycin, Tetracycline, Spectinomycin, Gentamycin, and Zeocin.

In some embodiments, a polynucleotide encoding a Cas9 nuclease as described herein (e.g., a polynucleotide encoding a Cas9 nuclease that is operably linked to an inducible or constitutive promoter) is in a separate expression cassette, expression vector, or plasmid than the other polynucleotide sequences disclosed herein (e.g., polynucleotides comprising a phenotype coding sequence or polynucleotides encoding a guide RNA). In some embodiments, a polynucleotide encoding a Cas9 nuclease as described herein is in the same expression cassette, vector, or plasmid as a polynucleotide comprising a phenotype coding sequence as described herein. In some embodiments, a polynucleotide encoding a Cas9 nuclease as described herein is stably integrated into the genomic material (e.g., chromosome) of a cell or organism as described herein.

### Lambda Red

In some embodiments, a lambda red recombinase system is used for making targeted changes as disclosed herein. Lambda red is a genetic engineering tool that enables homologous recombination in bacteria (“recombineering”). The lambda red system, which is derived from lambda bacteriophage, has three components: (1) lambda exonuclease (Exo), which digests the 5′ ended strand of dsDNA; (2) beta protein (Beta), which binds to ssDNA and enables strand annealing; and (3) gamma protein (Gam), which binds to the bacterial RecBCD enzyme and inhibits it from digesting linear DNA introduced into E. coli. The lambda red system is described in the art. See, e.g., Pyne et al., Applied and Environmental Microbiology, 2015, 81:5103-5114.

In some embodiments, the lambda red system (e.g., lambda red components Exo, Beta, and Gam) is integrated into the genome of an engineered bacterial cell or organism. In some embodiments, the lambda red system (e.g., lambda red components Exo, Beta, and Gam) is provided on a plasmid. In some embodiments, the lambda red system (e.g., lambda red components Exo, Beta, and Gam) is provided on a bacterial artificial chromosome.

In some embodiments, the lambda red system (e.g., lambda red components Exo, Beta, and Gam) is under constitutive expression, e.g., a polynucleotide encoding lambda red (e.g., lambda red components Exo, Beta, and Gam) is operably linked to a constitutively active promoter. In some embodiments, the lambda red system (e.g., lambda red components Exo, Beta, and Gam) is under inducible expression, e.g., a polynucleotide encoding lambda red (e.g., lambda red components Exo, Beta, and Gam) is operably linked to an inducible promoter. In some embodiments, an inducible promoter is a promoter that can be turned on and off by adding or removing an inducing agent. In some embodiments, an inducing agent is a molecule such as doxycycline, tetracycline, galactose, arabinose, rhamnose, metal ions, alcohol, or a steroid compound. In some embodiments, an inducible promoter is a promoter that is activated by environmental conditions, for example, light or temperature. In some embodiments, the promoter is a galactose inducible promoter. In some embodiments, the promoter is an arabinose inducible promoter. In some embodiments, the promoter is a rhamnose-inducible promoter. In some embodiments, the promoter is a doxycycline inducible promoter. In some embodiments, the inducer for expression of the lambda red system is the same as the inducer of another component of the engineered cell (e.g., the inducer for expression of the lambda red system is the same as an inducer that is used for inducing expression of Cas nuclease). In some embodiments, the inducer for expression of the lambda red system is different from the inducer of another component of the engineered cell (e.g., the inducer for expression of the lambda red system is different from an inducer that is used for inducing expression of Cas nuclease).

In some embodiments, lambda red recombineering is used with or without the CRISPR/Cas system to target and replace regions within genomic DNA. In some embodiments, the CRISPR/Cas system is used with or without lambda red recombineering to target and replace regions within genomic DNA. In some embodiments, the compositions, kits, and methods disclosed herein comprise both Cas9/CRISPR components and lambda red recombineering components for using lambda red recombineering in conjunction with Cas9/CRISPR for altering gene expression in cells and/or organisms. In some embodiments, the lambda red system provided on a separate plasmid or BAC from other gene editing components (e.g., on a separate plasmid or BAC as Cas9/CRISPR components). In some embodiments, the lambda red system provided on the same plasmid or BAC as other gene editing components (e.g., on the same plasmid or BAC as Cas9/CRISPR components).

### Genetic Regulatory Elements

In some embodiments, Cas9 and/or lambda red protein expression is regulated by genetic regulatory elements. Thus, in some embodiments, the engineered cells and/or organisms disclosed herein comprise components comprising one or more genetic regulatory elements. For example, inducible promoters typically have a certain level of “leakiness,” which can make tight regulation of proteins difficult to precisely regulate even in the absence of the inducing element. In order to have more precise regulation of the expression of Cas9 nuclease and the lambda red proteins, in some embodiments, inducible promoters (such as inducible promoters operably linked to Cas9 and lambda red) will have loxP or loxP variant sequences that flank a transcription terminator sequence directly following the promoter. In conjunction with an inducible promoter, transcription of Cas9 and lambda red ORFs are blocked by the insertion of transcription terminator sequences directly following the promoter sequence. In some embodiments, these termination sequences are flanked by loxP sites or loxP variants (e.g., lox2722). In some embodiments, these terminator sequences are flanked by FRT sites or FRT variants (e.g., F5). In some embodiments, Cre-recombinase or Flp recombinase expression cassettes are introduced into the engineered cells and/or organisms (e.g., bacteria, fungi, plant, or animal cell), causing the excision of the repressor elements and allowing for induction of transcription of polynucleotide sequences.

### Guide RNAs

In some embodiments, guide RNAs (“gRNAs”) are provided that target the phenotype coding sequences or the endogenous gene or genomic region to be modified. In general, a gRNA is an RNA sequence having sufficient complementarity with a target polynucleotide sequence to hybridize with the target sequence and direct sequence-specific binding of a CRISPR complex to the target sequence. In some embodiments, the degree of complementarity between a gRNA and its corresponding target sequence, when optimally aligned using a suitable alignment algorithm, is about or more than about 50%, 60%, 75%, 80%, 85%, 90%, 95%, 97.5%, 99%, or more. In some embodiments, a distinct gRNA is provided for each of the phenotype coding sequences and/or endogenous genes or genomic regions to be modified. Thus, in some embodiments, an engineered organism or engineered cell comprising a first phenotype coding sequence and a second phenotype coding sequence further comprises a polynucleotide sequence that comprises a first gRNA that targets the first phenotype coding sequence and a polynucleotide sequence that comprises a second gRNA that targets the second phenotype coding sequence.

In some embodiments, a gRNA comprises a sequence of at least about 15, 16, 17, 18, 19, 20, 21, 22, 23, 24, or 25 nucleotides in length (e.g., about 18-22 nucleotides in length or about 18-20 nucleotides in length) that is complementary to a portion of a phenotype coding sequence and further comprises a sequence that binds the Cas9 nuclease. In some embodiments, a gRNA comprises a nucleotide sequence that is complementary to a portion of a phenotype coding sequence that is adjacent to a Protospacer Adjacent Motif (PAM) sequence. In some embodiments, a gRNA further comprises a homologous donor polynucleotide sequence. In some embodiments, the homologous donor polynucleotide sequence comprises a restriction enzyme recognition site. In some embodiments, the homologous donor polynucleotide sequence has a length of at least 8 nucleotides, at least 10, 15, 20, 25, 50, 100, 200, 300, 400, 500, 600, 700, 800, 900, or 1000 nucleotides or more. In some embodiments, the homologous donor polynucleotide sequence has two arms with identical or substantially identical sequences adjacent to the gRNA target site with a specific mutation in between the two arms. The arms may be 20-1000 nucleotides or more and the mutation may be 1-1000 nucleotides or more.

Methods of designing gRNAs to target a sequence of interest and methods of optimizing gRNA structure to improve genomic editing efficiency are known in the art. See, e.g., Dang et al., Genome Biology, 2015, 16:280; Lee et al., Exp Physiol, 2017, doi: 10.1113/EP08604. In some embodiments, a gRNA is selected to reduce the degree of secondary structure within the gRNA. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62).

In some embodiments, the polynucleotide sequence comprising a gRNA that targets a phenotype coding sequence further comprises a heterologous promoter. In some embodiments, the promoter is a constitutively active promoter. Suitable constitutively active promoters include, but are not limited to, Tyrosine tRNA, Sigma 70 consensus sequence, TEF1, RPL18B, RNR2, TDH3, REV1, PGK, and ADH1.

In some embodiments, the polynucleotide sequence comprising a gRNA that targets a phenotype coding sequence further comprises a heterologous promoter. In some embodiments, the promoter is inducible. Suitable inducible promoters include, but are not limited to, arabinose-inducible (pBAD), galactose-inducible (GAL1), or rhamnose-inducible (pRha).

In some embodiments, the polynucleotide sequence comprising a gRNA that targets a phenotype coding sequence further comprises a sequence for an auxotrophic marker. In some embodiments, each polynucleotide sequence comprising a gRNA further comprises a sequence for an auxotrophic marker, wherein each gRNA has a different auxotrophic marker. In some embodiments, the auxotrophic marker is a yeast auxotrophic marker. Examples of auxotrophic markers include, but are not limited to, LEU2, URA3, LYS2 HIS3, MET17, and TRP1.

In some embodiments, the polynucleotide sequence comprising a gRNA that targets a phenotype coding sequence further comprises a sequence for an antibiotic resistance marker. In some embodiments, each polynucleotide sequence comprising a gRNA further comprises a sequence for an antibiotic resistance marker, wherein each gRNA has a different antibiotic resistance marker. Examples of antibiotic resistance markers include, but are not limited to, Ampicillin, Chloramphenicol, Kanamycin, Streptomycin, Tetracycline, Spectinomycin, Gentamycin, and Zeocin.

In some embodiments, each polynucleotide comprising a gRNA as described herein (e.g., a polynucleotide comprising a gRNA and further comprising a promoter and/or an auxotrophic or antibiotic marker) is in a separate expression cassette, expression vector, or plasmid than the other polynucleotide sequences disclosed herein (e.g., polynucleotides comprising other gRNA, polynucleotides comprising a phenotype coding sequence or polynucleotides encoding a guide RNA).

Components and Modifications for Alternative End-Joining and/or Non-Homologous End-Joining

In some embodiments, cells or organisms (e.g., bacteria) are engineered to express proteins that are components of the non-homologous end-joining (NHEJ) system. For example, in some embodiments bacteria are genetically engineered to express Mycobacterium tuberculosis Ku and Ligase D (LigD) proteins. These proteins allow bacteria to perform NHEJ, as described in Malyarchuk et al., DNA Repair, 2007, 6:1413-1424.

For example, in some embodiments, wild-type or genetically altered (e.g., Ku and LigD-expressing) bacteria that express chromogenic proteins or antibiotic resistance markers will have one or more of these coding sequences targeted and cleaved by Cas9. DSBs can be repaired through NHEJ, which is prone to error and will result in the loss of function of the targeted coding sequence. In some embodiments, changes in the frequency of loss of function of one or more coding sequences in wild-type versus genetically altered bacteria can be determined, e.g., in accordance with a method, system, or kit as disclosed herein.

In some embodiments, cells or organisms (e.g., bacteria) are engineered to express or overexpress proteins that are components of the alternative end-joining (A-EJ) system. For example, in some embodiments, bacteria are genetically engineered to express, or to express at higher levels, the components RecBCD and LigA. RecBCD is a nuclease/helicase complex that unwinds and degrades the DNA ends and is made up of three polypeptides: RecB, RecC, and RecD. LigA is DNA ligase that is responsible for alternative end-joining. An overview of the alternative end-joining repair mechanism and components is described, e.g., in Chayot et al., PNAS, 2010, 1017:2141-2146.

### IV. METHODS OF ALTERING GENE EXPRESSION

In another aspect, methods of altering gene expression are provided. In some embodiments, the method comprises culturing an engineered cell or organism as described herein (e.g., an engineered cell such as a yeast cell or an engineered organism such as yeast) to form a population of engineered cells or organisms, wherein the culturing is performed under conditions that result in expression of the Cas nuclease in at least one engineered cell or organism, wherein the Cas nuclease cleaves an endogenous gene, genomic region, or phenotype coding sequence; thereby altering gene expression in at least one engineered cell or organism.

In some embodiments, the method of altering gene expression comprises:

providing a cell or organism as described herein (e.g., a prokaryotic or eukaryotic cell such as a yeast cell, mammalian cell, insect cell, or plant cell, or a microbial organism or eukaryotic organism as described herein);

transforming the cell or organism with one or more polynucleotide sequences as described herein (e.g., a first heterologous polynucleotide sequence comprising a first phenotype coding sequence operably linked to a first promoter, a second heterologous polynucleotide sequence comprising a second phenotype coding sequence operably linked to a second promoter, a third heterologous polynucleotide sequence comprising a third promoter operably linked to a polynucleotide encoding a Cas nuclease, a fourth heterologous polynucleotide sequence comprising a first gRNA that targets the first phenotype coding sequence, and a fifth heterologous polynucleotide sequence comprising a second gRNA that targets the second phenotype coding sequence) to express the polynucleotide sequences; and

culturing the transformed cell or organism to form a population of cells or organisms, wherein the culturing is performed under conditions that result in expression of the Cas nuclease in at least one cell or organism, wherein the Cas nuclease cleaves a phenotype coding sequence (e.g., one or more of the first phenotype coding sequence and the second phenotype coding sequence); thereby altering gene expression in at least one cell or organism.

In some embodiments, wherein the promoter that is operably linked to the polynucleotide encoding the Cas nuclease (e.g., Cas9 nuclease) is an inducible promoter, the culturing is in the presence of an inducer to induce expression of the Cas nuclease in at least one engineered cell or organism. In some embodiments, the polynucleotide encoding Cas nuclease is operably linked to a galactose-inducible promoter and the method comprises culturing the engineered cells or organisms in the presence of galactose to induce the expression of Cas. In some embodiments, the polynucleotide encoding Cas nuclease is operably linked to an arabinose-inducible promoter and the method comprises culturing the engineered cells or organisms in the presence of arabinose to induce the expression of Cas. In some embodiments, the polynucleotide encoding Cas nuclease is operably linked to a rhamnose-inducible promoter and the method comprises culturing the engineered cells or organisms in the presence of rhamnose to induce the expression of Cas.

In some embodiments, wherein a polynucleotide comprising a gRNA is operably linked to a promoter that is an inducible promoter, the culturing is in the presence of an inducer to induce expression of the gRNA in at least one engineered cell or organism. In some embodiments, the polynucleotide comprising the gRNA is operably linked to a galactose-inducible promoter and the method comprises culturing the engineered cells or organisms in the presence of galactose to induce the expression of the gRNA. In some embodiments, the polynucleotide comprising the gRNA is operably linked to an arabinose-inducible promoter and the method comprises culturing the engineered cells or organisms in the presence of arabinose to induce the expression of the gRNA. In some embodiments, the polynucleotide comprising the gRNA is operably linked to a rhamnose-inducible promoter and the method comprises culturing the engineered cells or organisms in the presence of rhamnose to induce the expression of the gRNA. In some embodiments, the polynucleotide comprising the gRNA is operably linked to a constitutively active promoter, and the gRNA is constitutively expressed.

Means of transforming cells and organisms are known in the art. In some embodiments, transforming the cell or organism with the polynucleotide sequences as described herein (e.g., donor DNA, gRNA, phenotype coding sequence polynucleotides, Cas nuclease polynucleotides or expression cassettes, or lambda red polynucleotides or expression cassettes) comprises delivering the polynucleotide sequences through chemical transformation of plasmids or electroporation. In some embodiments, transforming the cell or organism with the polynucleotide sequences as described herein (e.g., donor DNA, gRNA, phenotype coding sequence polynucleotides, Cas nuclease polynucleotides or expression cassettes, or lambda red polynucleotides or expression cassettes) comprises delivering the polynucleotide sequences through transformation by silicon-carbide whiskers.

In some embodiments, the method further comprises screening the population of engineered cells or organisms to identify at least one engineered cell that exhibits a change in phenotype as compared to the phenotype of the engineered cell or organism prior to the culturing step. In some embodiments, the method further comprises screening the population of engineered cells or organisms (e.g., engineered microbial organisms) to identify at least one engineered cell or organism that exhibits a color change as compared to the color of the engineered cell or organism prior to the culturing step. For example, as detailed in Example 1 below, in some embodiments, the engineered cell or organism comprises chromogenic coding sequences for blue, red, and yellow genes. In the absence of Cas (e.g., Cas9) nuclease expression, each of these chromogenic proteins are expressed, causing the cell or organism to have a brown phenotype. In the presence of an inducer (e.g., galactose), one or more of the chromogenic coding sequences is disrupted due to the activity of Cas, which results in a loss of expression of one or more of the chromogenic proteins and changes the color phenotype of the cell or organism.

In some embodiments, the method of altering gene expression comprises disrupting expression of a detectable phenotype as described herein (e.g., disrupting expression of a chromogenic protein). In some embodiments, the method of altering gene expression comprises restoring expression of a detectable phenotype by repairing a phenotype coding expression to permit expression of a functional protein having a detectable phenotype. In some embodiments, the method of altering gene expression comprises a combination of disrupting expression of one detectable phenotype and restoring expression of another detectable phenotype.

In some embodiments, a first phenotype coding sequence encodes a functional first protein having a first detectable phenotype and the Cas (e.g., Cas9) nuclease cleaves the first phenotype coding sequence and disrupts expression of the first protein. In some embodiments, a first phenotype sequence comprises an engineered disruption in a sequence encoding a first protein that prevents expression of the first detectable phenotype, and the method comprises cleaving the first coding sequence at the engineered disruption with the Cas (e.g., Cas9) nuclease and repairing the first coding sequence to permit expression of the first protein.

In some embodiments, a second phenotype coding sequence encodes a functional second protein having a second detectable phenotype and the Cas (e.g., Cas9) nuclease cleaves the second phenotype coding sequence and disrupts expression of the second protein. In some embodiments, a second phenotype sequence comprises an engineered disruption in a sequence encoding a second protein that prevents expression of the second detectable phenotype, and the method comprises cleaving the second coding sequence at the engineered disruption with the Cas (e.g., Cas9) nuclease and repairing the second coding sequence to permit expression of the second protein.

In some embodiments, the first phenotype coding sequence encodes a functional first protein having a first detectable phenotype and the method comprises cleaving the first phenotype coding sequence with the Cas (e.g., Cas9) nuclease and disrupting expression of the first protein; and the second phenotype sequence comprises an engineered disruption in a sequence encoding a second protein that prevents expression of the second detectable phenotype, and the method comprises cleaving the second coding sequence at the engineered disruption with the Cas (e.g., Cas9) nuclease and repairing the second coding sequence to permit expression of the second protein.

In some embodiments, the first phenotype sequence comprises an engineered disruption in a sequence encoding a first protein that prevents expression of the first detectable phenotype, and the method comprises cleaving the first coding sequence at the engineered disruption with the Cas (e.g., Cas9) nuclease and repairing the first coding sequence to permit expression of the first protein; and the second phenotype coding sequence encodes a functional second protein having a second detectable phenotype and the method comprises cleaving the second phenotype coding sequence with the Cas (e.g., Cas9) nuclease and disrupting expression of the second protein.

In some embodiments, e.g., as shown in Example 7 or Example 9 below, the engineered cell or organism comprises a phenotype coding sequence encoding a functional first protein having a detectable phenotype (e.g., a GFP protein), and the method comprises cleaving the coding sequence with the Cas (e.g., Cas9) nuclease, thereby disrupting expression of the first protein. The engineered disruption is defined by the sequence in the homologous repair donor sequence.

In some embodiments, e.g., as shown in Example 8 or Example 10, the engineered cell or organism comprises a phenotype coding sequence encoding a functional first protein having a detectable phenotype (e.g., a GFP protein), and the method comprises cleaving the coding sequence with the Cas (e.g., Cas9) nuclease and modifies the expression of the first protein, e.g., to instead express a second phenotype (e.g., YFP or BFP). The engineered changes are defined by the sequence in the homologous repair donor sequence.

Counterselection with Auxotrophic Markers

In some embodiments, the cell or organism comprises one or more polynucleotide sequences that comprises an auxotrophic marker, and the method further comprises: culturing the population of engineered cells or organisms in the presence of one or more counterselection agents for the auxotrophic marker(s); and selecting for one or more engineered cells or organisms that do not express the polynucleotide sequence associated with the auxotrophic marker being counterselected; thereby preventing expression of the gRNA associated with the auxotrophic marker being counterselected and preventing alteration of the chromogenic coding sequence targeted by the gRNA associated with the auxotrophic marker being counterselected.

As a non-limiting example, an organism comprising a plasmid that comprises a first gRNA that targets a first chromogenic coding sequence and the URA3 auxotrophic marker can be counterselected by culturing the organism in the presence of 5-FOA (e.g., by growing the organism on media containing 5-FOA). As a result, the first gRNA will not be expressed and the first chromogenic coding sequence will not be targeted for gene editing. An organism comprising a plasmid that comprises a second gRNA that targets a second chromogenic coding sequence and the LYS2 auxotrophic marker can be counterselected by culturing the organism in the presence of alpha-adipic acid (e.g., growing the organism on media containing alpha-adipic acid). As a result, the second gRNA will not be expressed and the second chromogenic coding sequence will not be targeted for gene editing.

Selection with Antibiotic Resistance Markers

In some embodiments, the cell or organism comprises one or more polynucleotide sequences that comprises an antibiotic resistance marker, and the method further comprises culturing the population of engineered cells or organisms in the presence of one or more selection agents for the antibiotic resistance marker(s).

### Lambda Red Recombineering

In some embodiments, the methods of altering gene expression comprise the use of the lambda red system for targeting and replacing a gene or genomic region (e.g., a phenotype coding region as disclosed herein that is introduced into the cell or organism, or an endogenous gene or region within the genome of the cell or organism). Lambda red is a genetic engineering tool that enables homologous recombination in bacteria (“recombineering”). The lambda red system, which is derived from lambda bacteriophage, has three components: (1) lambda exonuclease (Exo), which digests the 5′ ended strand of dsDNA; (2) beta protein (Beta), which binds to ssDNA and enables strand annealing; and (3) gamma protein (Gam), which binds to the bacterial RecBCD enzyme and inhibits it from digesting linear DNA introduced into E. coli. The lambda red system is described in the art. See, e.g., Pyne et al., Applied and Environmental Microbiology, 2015, 81:5103-5114.

In some embodiments, the method of altering gene expression comprises:

providing a cell or organism as described herein (e.g., a prokaryotic or eukaryotic cell such as a yeast cell, mammalian cell, insect cell, or plant cell, or a microbial organism or eukaryotic organism as described herein);

transforming the cell or organism with one or more polynucleotide sequences as described herein (e.g., a heterologous polynucleotide sequence comprising a phenotype coding sequence operably linked to a promoter, a heterologous polynucleotide sequence comprising a promoter operably linked to a polynucleotide encoding lambda red, and a heterologous polynucleotide sequence comprising a homologous donor DNA sequence); and

culturing the transformed cell or organism to form a population of cells or organisms, wherein the culturing is performed under conditions that result in expression of the lambda red in at least one cell or organism, wherein the lambda red catalyzes the homologous recombination of a donor DNA sequence at an endogenous gene, genomic region, or phenotype coding sequence in the engineered cell; thereby altering gene expression in at least one cell or organism.

In some embodiments, wherein the promoter that is operably linked to the polynucleotide encoding the lambda red is an inducible promoter, the culturing is in the presence of an inducer to induce expression of the lambda red in at least one engineered cell or organism. In some embodiments, the polynucleotide encoding lambda red is operably linked to a galactose-inducible promoter and the method comprises culturing the engineered cells or organisms in the presence of galactose to induce the expression of lambda red. In some embodiments, the polynucleotide encoding lambda red is operably linked to an arabinose-inducible promoter and the method comprises culturing the engineered cells or organisms in the presence of arabinose to induce the expression of lambda red. In some embodiments, the polynucleotide encoding lambda red nuclease is operably linked to a rhamnose-inducible promoter and the method comprises culturing the engineered cells or organisms in the presence of rhamnose to induce the expression of lambda red.

In some embodiments, the methods disclosed herein comprise the use of lambda red recombineering with or without the CRISPR/Cas system. In some embodiments, the methods disclosed herein comprise the use of the CRISPR/Cas system with or without lambda red recombineering. Without being bound to a particular theory, it is expected that inclusion of homologous recombination by lambda red will increase the efficiency of CRISPR/Cas9-mediated gene editing in bacterial cells. Thus, in some embodiments, a method or module for altering gene expression comprises using lambda red recombineering in both the absence and presence of the CRISPR/Cas system (e.g., Cas9 nuclease and gRNA components as disclosed herein) and comparing the rate of gene targeting in the absence of Cas9 nuclease and gRNA to the rate of gene targeting in the presence of Cas9 nuclease and gRNA.

### Targeting Endogenous or Genomic Targets

In some embodiments, the methods of altering gene expression comprise targeting an endogenous phenotype or a genomic locus (e.g., a gene or a genomic region of the cell or organism). For example, in some embodiments, the CRISPR/Cas system and/or lambda red recombineering system is used in an engineered cell or organism as disclosed herein in order to target an endogenous gene or genomic region in the genome of the cell or organism. In some embodiments, the targeting comprises disrupting the function of an endogenous gene or genomic region in the genome of the cell or organism, e.g., by introducing a polynucleotide sequence that comprises a stop codon or other sequence that prevents the expression of a functional protein, or by introducing a polynucleotide sequence that comprises an open reading frame for a different gene in order to change the function of the endogenous gene. In some embodiments, the targeting comprises restoring the function of a disrupted endogenous gene or genomic region in the genome of the cell or organism, e.g., by introducing a polynucleotide sequence that repairs a broken gene or genomic region (e.g., promoter) or a prematurely terminated gene or genomic region in order to restore the native function of the gene or genomic region.

In some embodiments, the method of altering gene expression comprises:

providing a cell or organism as described herein (e.g., a prokaryotic or eukaryotic cell such as a yeast cell, mammalian cell, insect cell, or plant cell, or a microbial organism or eukaryotic organism as described herein);

transforming the cell or organism with one or more polynucleotide sequences as described herein (e.g., a heterologous polynucleotide sequence comprising a promoter operably linked to a polynucleotide encoding a Cas nuclease, a heterologous polynucleotide sequence comprising a promoter operably linked to a polynucleotide encoding lambda red, one or more heterologous polynucleotide sequences comprising a gRNA that targets the endogenous gene or genomic region, and/or one or more donor DNAs) to express the polynucleotide sequences; and

culturing the transformed cell or organism to form a population of cells or organisms, wherein the culturing is performed under conditions that result in expression of the Cas nuclease in at least one cell or organism, wherein the Cas nuclease cleaves at the endogenous gene or genomic region; thereby altering gene expression in at least one cell or organism.

In some embodiments, the method of altering gene expression comprises targeting an endogenous gene or genomic region that is functional (e.g., exhibits a detectable phenotype, such as a detectable color, fluorescence, scent, enzymatic activity, antibiotic resistance, or morphology) and transforming the cell or organism with a donor DNA sequence that disrupts the endogenous gene or genomic region (e.g., a sequence that results in a premature termination codon), thereby altering gene expression in the cell or organism.

In some embodiments, the method of altering gene expression comprises targeting an endogenous gene or genomic region that is functional (e.g., exhibits a detectable phenotype, such as a detectable color, fluorescence, scent, enzymatic activity, antibiotic resistance, or morphology) and transforming the cell or organism with a donor DNA sequence that encodes a different function (e.g., a sequence comprising an ORF for a different gene, such as an antibiotic resistance cassette) to replace the endogenous function of the targeted gene or genomic region with a new function, thereby altering gene expression in the cell or organism.

In some embodiments, the method of altering gene expression comprises targeting an endogenous gene or genomic region that is non-functional (e.g., due to the presence of a polynucleotide sequence that results in a premature termination codon) and transforming the cell or organism with a donor DNA sequence that restores the function of the gene or genomic region, thereby altering gene expression in the cell or organism.

### V. KITS

In another aspect, kits comprising the polynucleotides, expression cassettes, expression vectors, plasmids, and/or cells as described herein are provided. In some embodiments, the kit comprises:

one or more polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype;

a polynucleotide sequence comprising a promoter operably linked to a polynucleotide encoding a Cas nuclease; and

a polynucleotide sequence comprising a guide RNA (gRNA) that targets the phenotype coding sequence.

In some embodiments, the kit further comprises one or more polynucleotide sequences comprising a homologous donor DNA sequence (e.g., dsDNA or ssDNA donor sequence).

In some embodiments, the kit comprises:

a first polynucleotide sequence comprising a first chromogenic coding sequence operably linked to a first promoter, wherein the first chromogenic coding sequence either (i) encodes a functional first chromogenic protein or (ii) comprises an engineered disruption in a sequence encoding a first chromogenic protein that prevents expression of the first chromogenic protein;

a second polynucleotide sequence comprising a second chromogenic coding sequence operably linked to a second promoter, wherein the second chromogenic coding sequence either (i) encodes a functional second chromogenic protein or (ii) comprises an engineered disruption in a sequence encoding a second chromogenic protein that prevents expression of the second chromogenic protein;

a third polynucleotide sequence comprising an inducible promoter operably linked to a polynucleotide encoding a Cas nuclease;

a fourth polynucleotide sequence comprising a first guide RNA (gRNA) that targets the first chromogenic coding sequence; and

a fifth polynucleotide sequence comprising a second gRNA that targets the second chromogenic coding sequence.

In some embodiments, the kit comprises:

an engineered cell or organism comprising an endogenous gene or genomic region to be targeted for gene alteration, or one or more heterologous polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype; and (a) a heterologous polynucleotide sequence comprising a promoter operably linked to a polynucleotide encoding a Cas nuclease and a heterologous polynucleotide sequence comprising a guide RNA (gRNA) that targets the endogenous gene, genomic region, or phenotype coding sequence; and/or (b) a heterologous polynucleotide sequence comprising a promoter operably linked to a polynucleotide encoding lambda red.

In some embodiments, the kit comprises an engineered cell or organism comprising an endogenous gene or genomic region to be targeted for gene alteration, or one or more heterologous polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype, and further comprising one or both of a Cas nuclease and a lambda red system integrated into the genome of the engineered cell or organism. In some embodiments, the engineered cell or organism comprises one of the Cas nuclease or the lambda red system integrated into the genome of the engineered cell or organism, and the kit further comprises a heterologous polynucleotide sequence encoding the other of the Cas nuclease or the lambda red system. In some embodiments, the kit further comprises one or more polynucleotide sequences comprising a homologous donor DNA sequence (e.g., dsDNA or ssDNA donor sequence).

In some embodiments, the kit comprises:

an engineered cell or organism comprising one or more heterologous polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype; and

one or more of a heterologous polynucleotide sequence comprising a promoter operably linked to a polynucleotide encoding a Cas nuclease, a heterologous polynucleotide sequence comprising a gRNA that targets the phenotype coding sequence, and a heterologous polynucleotide sequence comprising a homologous donor DNA sequence.

In some embodiments, a kit is for use in targeting an endogenous phenotype or a genomic locus of an engineered cell or organism and comprises:

an engineered cell or organism (e.g., a prokaryotic or eukaryotic cell such as a yeast cell, mammalian cell, insect cell, or plant cell, or a microbial organism or eukaryotic organism as described herein) that comprises an endogenous gene or genomic region to be targeted;

a polynucleotide sequence comprising a promoter operably linked to a polynucleotide encoding a Cas nuclease;

a polynucleotide sequence comprising a gRNA that targets the endogenous gene or genomic region of the engineered cell or organism; and

one or more polynucleotide sequences comprising a homologous donor DNA sequence (e.g., dsDNA or ssDNA donor sequence).

In some embodiments of the kits disclosed herein, the kit further comprises a heterologous polynucleotide sequence comprising a promoter operably linked to a polynucleotide encoding lambda red.

In some embodiments, a kit that is for use in targeting an endogenous phenotype or a genomic locus of an engineered cell or organism comprises an engineered cell or organism that comprises an endogenous gene or genomic region to be targeted and one or both of a Cas nuclease and a lambda red system integrated into the genome of the engineered cell or organism. In some embodiments, the engineered cell or organism comprises one of the Cas nuclease or the lambda red system integrated into the genome of the engineered cell or organism, and the kit further comprises a heterologous polynucleotide sequence encoding the other of the Cas nuclease or the lambda red system. In some embodiments, the kit further comprises one or more polynucleotide sequences comprising a homologous donor DNA sequence (e.g., dsDNA or ssDNA donor sequence).

In some embodiments, the kit further comprises one or more reagents for transforming the one or more heterologous polynucleotide sequences into the engineered cell or organism. In some embodiments, the kit further comprises one or more reagents for inducing expression of the Cas nuclease, inducing expression of the gRNA, or preventing expression of the gRNA.

In some embodiments, the polynucleotide sequences (e.g., as described in Section III above) are in one or more expression cassettes, expression vectors, and/or plasmids. In some embodiments, the kit further comprises one or more cells or organisms (e.g., microbial, prokaryotic, protist, or eukaryotic cells or organisms as described herein, e.g., as in Section III above). In some embodiments, the kit comprises a prokaryotic cell. In some embodiments, the kit comprises a protist cell. In some embodiments, the kit comprises a eukaryotic cell.

In some embodiments, the polynucleotide sequences, expression cassettes, expression vectors, and/or plasmids are in a cell or organism as described herein. Thus, in another aspect, kits are provided that comprise an engineered cell or engineered organism (e.g., an engineered microbial organism) as described herein. In some embodiments, the engineered microbial organism is fungal (e.g., yeast). In some embodiments, the engineered microbial organism is prokaryotic (e.g., bacterial). In some embodiments, the engineered microbial organism is eukaryotic (e.g., yeast, nematode, or plant).

In some embodiments, a microbial organism, polynucleotide sequence, expression cassette, expression vector, and/or plasmid that is provided in the kit is in lyophilized form. In some embodiments, the lyophilized microbial organism, polynucleotide sequence, expression cassette, expression vector, and/or plasmid is reconstituted by the end user of the kit. Thus, in some embodiments, the kit further comprises instructions for reconstituting the lyophilized organism, polynucleotide sequence, expression cassette, expression vector, and/or plasmid.

In some embodiments, a microbial organism and polynucleotide sequences, expression cassettes, expression vectors, and/or plasmids are provided separately in the kit. In some embodiments, a microbial organism and polynucleotide sequences, expression cassettes, expression vectors, and/or plasmids are provided separately in the kit in lyophilized form.

In some embodiments, the kit further comprises one or more culturing reagents. In some embodiments, the kit further comprises one or more transfection reagents. In some embodiments, the kit further comprises one or more transformation reagents. In some embodiments, the kit further comprises one or more reagents comprising culture medium (e.g., liquid medium), selective medium, solid plating medium, media supplements, plates, tubes, loops, or other plasticware. In some embodiments, the kit further comprises an inducer for the inducible promoter. In some embodiments, the kit comprises galactose. In some embodiments, the kit comprises arabinose. In some embodiments, the kit comprises rhamnose. In some embodiments, the kit comprises counterselection agents for the auxotrophic markers as described herein (e.g., 5-fluoro-orotic acid or alpha-adipic acid). In some embodiments, the kit comprises selection agents for the antibiotic resistance markers as described herein (e.g., Ampicillin, Kanamycin, Spectinomycin, Gentamycin, or Zeocin).

In some embodiments, the kit further comprises one or more reagents for detecting the genotype of the engineered cell or engineered microbial organism, wherein the one or more reagents comprise DNA polymerases, primers, dNTPs, restriction enzymes, or buffers. In some embodiments, the kit further comprises equipment for transforming, culturing, detecting phenotypes, or genotyping a cell or organism. For example, in some embodiments, the kit comprises antibodies, western detection reagents, protein transfer equipment, pipets, pipettors, incubators, incubator shakers, thermal cyclers, DNA electrophoresis equipment, power supplies, waterbaths, sequencing materials, or bioinformatics software.

In some embodiments, the kit further comprises instruction materials containing directions (i.e., protocols) for the practice of the methods described herein (e.g., for altering gene expression in a cell or organism as described herein). In some embodiments, the kit comprises instructions for analyzing a cell or organism as described herein (e.g., for inducing Cas9 activity, detecting one more changes in a detectable phenotype, and/or analyzing a genotype for a cell or organism as described herein). In some embodiments, the kit comprises instructions for designing gRNAs and other CRISPR/Cas components as described herein. In some embodiments, the kit comprises a curriculum (e.g., a curriculum for educators) for using the cells, compositions, kits, and/or methods as disclosed herein for teaching fundamental principles in gene editing.

While the instructional materials typically comprise written or printed materials they are not limited to such. Any medium capable of storing such instructions and communicating them to an end user is contemplated by this disclosure. Such media include, but are not limited to, electronic storage media (e.g., magnetic discs, tapes, chips, etc.), optical media (e.g., CD-ROM), and the like. Such media may also include addresses to internet sites that provide such instructional materials.

### VI. SYSTEMS AND METHODS FOR EDUCATION AND TRAINING

In another aspect, systems and methods for education and training in the core processes of gene expression and gene editing are provided. In some embodiments, the polynucleotide sequences, expression cassettes, expression vectors, plasmids, cells, organisms, and reagents disclosed herein are provided as a modular system. The system can be formulated as a series of modules that collectively represent the various stages in gene expression and alteration of gene expression in a cell or organism, with each stage or module containing a full set of reagents, media, and other disposable components, as well as an instruction manual with directions for performing standard procedures for each stage, and in some embodiments, an assessment module for evaluating the users' understanding of the processes taught. Certain modules can be sold or purchased independently of the system as a whole, such as for the purposes of replenishing a supply for a particular stage, offering the instructor or trainee a choice at the point of use, or adapting the system to smaller or larger numbers of trainees.

For example, in some embodiments, the system can comprise one or more of the following modules.

Module for expressing a detectable phenotype in a cell or organism: In some embodiments, a module for expressing one or more detectable phenotypes (e.g., detectable colors and/or fluorescences; detectable scents; detectable enzymatic activities; resistance to an antibiotic; or detectable morphologies) in a cell or organism is provided. In some embodiments, the module comprises a set of reagents and materials for providing, generating, or culturing a cell or organism that expresses a detectable phenotype. In some embodiments, the module comprises one or more of a cell or organism (e.g., a prokaryotic or eukaryotic cell or organism), one or more heterologous polynucleotides comprising a phenotype coding sequence operably linked to a promoter, a heterologous polynucleotide sequence comprising a promoter operably linked to a polynucleotide encoding a Cas nuclease, a heterologous polynucleotide sequence comprising a promoter operably linked to a polynucleotide encoding lambda red, a heterologous polynucleotide sequence comprising a gRNA that targets the chromogenic coding sequence, and a heterologous polynucleotide sequence comprising a homologous donor DNA sequence, as described herein. In some embodiments, the module comprises a cell or organism comprising one or more heterologous polynucleotides comprising a phenotype coding sequence operably linked to a promoter, and further comprises one or more heterologous polynucleotides (e.g., heterologous polynucleotide sequence comprising a promoter operably linked to a polynucleotide encoding a Cas nuclease, a heterologous polynucleotide sequence comprising a promoter operably linked to a polynucleotide encoding lambda red, a heterologous polynucleotide sequence comprising a gRNA that targets the chromogenic coding sequence, and a heterologous polynucleotide sequence comprising a homologous donor DNA sequence) for transforming into the cell or organism. In some embodiments, the module comprises or more culturing reagents, transfection reagents, and/or transformation reagents.

Module for expressing a broken gene in a cell or organism: In some embodiments, a module for expressing one or more phenotype coding sequences comprising an engineered disruption in a protein-encoding sequence or promoter region that prevents expression of a detectable phenotype (e.g., a broken gene such as a broken chromogenic gene) in a cell or organism is provided. In some embodiments, the module comprises a set of reagents and materials for providing, generating, or culturing a cell or organism having a broken gene. In some embodiments, the module comprises one or more of a cell or organism (e.g., a prokaryotic or eukaryotic cell or organism), one or more heterologous polynucleotides operably linked to a promoter, wherein the polynucleotide comprises an engineered disruption in a sequence encoding a protein that prevents expression of the detectable phenotype, a heterologous polynucleotide sequence comprising a promoter operably linked to a polynucleotide encoding a Cas nuclease, a heterologous polynucleotide sequence comprising a promoter operably linked to a polynucleotide encoding lambda red, a heterologous polynucleotide sequence comprising a gRNA that targets the chromogenic coding sequence, and a heterologous polynucleotide sequence comprising a homologous donor DNA sequence, as described herein. In some embodiments, the module comprises a cell or organism comprising one or more heterologous polynucleotides comprising an engineered disruption in a sequence encoding a protein or promoter region that prevents expression of a detectable phenotype (e.g., a broken gene such as a broken chromogenic gene), and further comprises one or more heterologous polynucleotides (e.g., heterologous polynucleotide sequence comprising a promoter operably linked to a polynucleotide encoding a Cas nuclease, a heterologous polynucleotide sequence comprising a promoter operably linked to a polynucleotide encoding lambda red, a heterologous polynucleotide sequence comprising a gRNA that targets the chromogenic coding sequence, and a heterologous polynucleotide sequence comprising a homologous donor DNA sequence) for transforming into the cell or organism. In some embodiments, the module comprises or more culturing reagents, transfection reagents, and/or transformation reagents.

Module for gene editing: In some embodiments, a module for altering expression of a detectable phenotype (e.g., to change the detectable phenotype into a second detectable phenotype or to disrupt expression of the detectable phenotype) or for repairing a broken gene (e.g., repairing a broken gene such that a detectable phenotype is expressed) is provided. In some embodiments, the module comprises a set of reagents and materials for altering and/or repairing gene expression. In some embodiments, the module comprises a heterologous polynucleotide sequence comprising a promoter (e.g., an inducible promoter) operably linked to a polynucleotide encoding a Cas nuclease, a heterologous polynucleotide sequence comprising a promoter operably linked to a polynucleotide encoding lambda red, a heterologous polynucleotide sequence comprising a gRNA that targets the chromogenic coding sequence, and/or a heterologous polynucleotide sequence comprising a homologous donor DNA sequence. In some embodiments, the module comprises one or more reagents for inducing expression of a Cas nuclease and/or the lambda red system or for inducing or preventing the expression of a gRNA, e.g., an inducer for an inducible promoter or a selection agent or counterselection agent for an antibiotic or auxotrophic marker. In some embodiments, the module comprises or more culturing reagents, transfection reagents, and/or transformation reagents.

Module for endogenous or genomic targeting: In some embodiments, a module for targeting an endogenous gene or genomic region in a cell or organism (e.g., disrupting the function of the endogenous gene or genomic region, replacing the function of the endogenous gene or genomic region with a new function, or restoring the function of the endogenous gene or genomic region) is provided. In some embodiments, the module comprises a set of reagents and materials for providing, generating, or culturing a cell or organism that expresses an endogenous gene or genomic region to be targeted. In some embodiments, the module comprises one or more of a cell or organism (e.g., a prokaryotic or eukaryotic cell or organism) comprising an endogenous gene or genomic region to be targeted, a heterologous polynucleotide sequence comprising a promoter operably linked to a polynucleotide encoding a Cas nuclease, a heterologous polynucleotide sequence comprising a promoter operably linked to a polynucleotide encoding lambda red, a heterologous polynucleotide sequence comprising a gRNA that targets the endogenous gene or genomic region, and a heterologous polynucleotide sequence comprising a homologous donor DNA sequence, as described herein. In some embodiments, the module comprises a cell or organism comprising an endogenous gene or genomic region to be targeted, and further comprises one or more heterologous polynucleotides (e.g., heterologous polynucleotide sequence comprising a promoter operably linked to a polynucleotide encoding a Cas nuclease, a heterologous polynucleotide sequence comprising a promoter operably linked to a polynucleotide encoding lambda red, a heterologous polynucleotide sequence comprising a gRNA that targets the endogenous gene or genomic region, and a heterologous polynucleotide sequence comprising a homologous donor DNA sequence) for transforming into the cell or organism. In some embodiments, the module comprises or more culturing reagents, transfection reagents, and/or transformation reagents.

Module for analyzing the cell or organism: In some embodiments, a module for detecting a phenotype, a change in phenotype, and/or a genotype of a cell or organism following a gene editing event is provided. In some embodiments, the module comprises a set of reagents and materials for detecting or analyzing a phenotype or genotype. For example, in some embodiments, the module comprises one or more reagents for detecting the genotype of an engineered cell or engineered microbial organism, e.g., DNA polymerases, primers, dNTPs, restriction enzymes, or buffers. In some embodiments, the module comprises equipment for transforming, culturing, detecting phenotypes, or genotyping an engineered cell or organism.

Assessment module: In some embodiments, an assessment module containing materials for use by educators or trainers can also be included or otherwise made available. The assessment module might serve the purpose of aiding educators or trainers in evaluating a trainee's understanding of the purpose and use of the materials and procedures, and in identifying particular areas in which the trainee needs improvement or needs to pay greater attention to details such as timing, operating conditions, proper use of equipment, or other aspects of the procedures.

In some embodiments, the system comprises an instruction manual containing the entire procedure, including all steps to be followed for each module and procedures for using each piece of equipment and each reagent in each module, as well as the results to be expected from each procedure when the procedures are performed correctly.

### VII. EXAMPLES

The following examples are provided to illustrate, but not to limit, the claimed invention.

### Example 1

### Kaleidoscope Organism Containing Multiple Color Genes

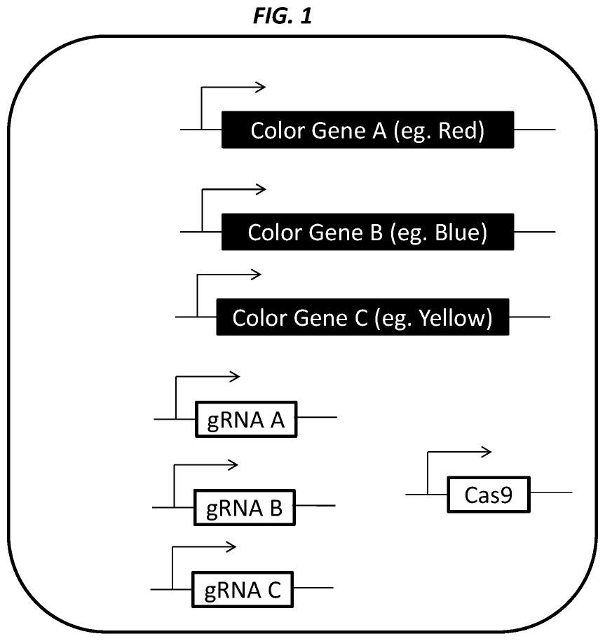

The organism can contain and express multiple color genes. Depending on which of these genes are knocked out, the organism's phenotype will change colors. As shown in FIG. 1, an organism (e.g., E. coli or yeast) contains multiple color genes, and the genes necessary for targeted knock out of each of these genes (e.g., Cas9 system with color gene specific guide RNAs). These genetic elements can be chromosomal or on plasmid(s). In the case of three color genes (e.g., Red, Blue and Yellow), the organism will be brownish when all three genes are expressed. If the Cas9 system and one or more of the gRNA's are expressed then the color of the organism will change due to the knock-out of one or more of the color genes.
For example:
Color Genes A+B+C expressed=Brown Phenotype (blue, red and yellow genes expressed)
Color Genes A+B+C expressed+Cas9 expressed+gRNA A expressed [symbol] Green Phenotype (blue and yellow genes expressed)
Color Genes A+B+C expressed+Cas9 expressed+gRNA B expressed [symbol] Orange Phenotype (red and yellow genes expressed)
Color Genes A+B+C expressed+Cas9 expressed+gRNA C expressed [symbol] Purple Phenotype (blue and red genes expressed)
Color Genes A+B+C expressed+Cas9 expressed+gRNA A and gRNA B expressed [symbol] Yellow Phenotype (yellow gene expressed)
Color Genes A+B+C expressed+Cas9 expressed+gRNA A and gRNA C expressed [symbol] Blue Phenotype (blue gene expressed)
Color Genes A+B+C expressed+Cas9 expressed+gRNA C and gRNA B expressed [symbol] Red Phenotype (red gene expressed)
Color Genes A+B+C expressed+Cas9 expressed+gRNA A, gRNA B and gRNA C expressed [symbol] white or beige Phenotype (no color genes expressed)

### Example 2

### Yeast Version 1

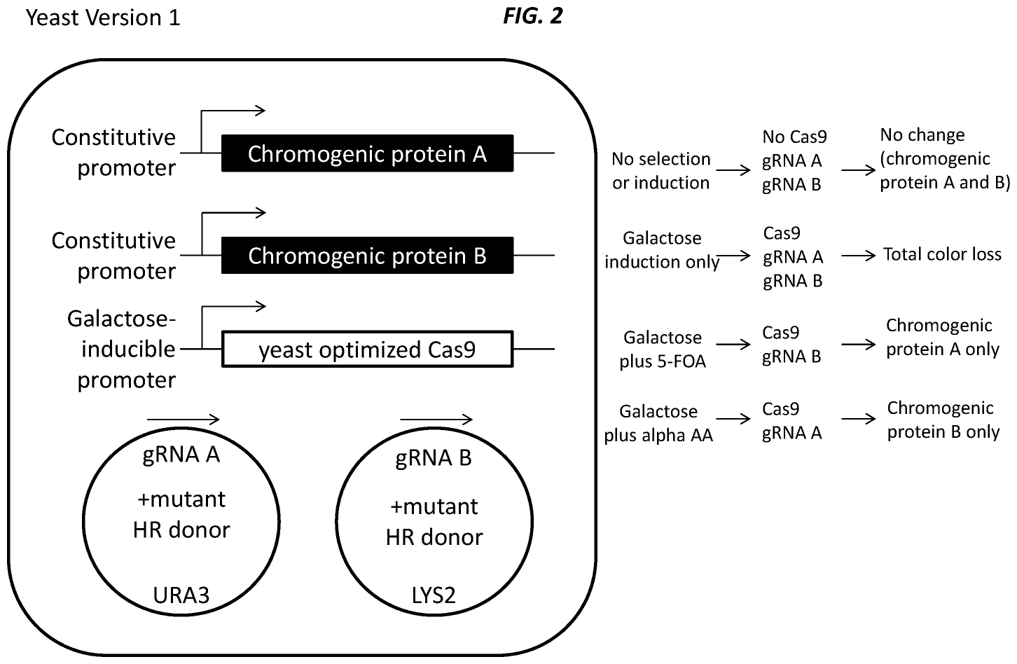

A schematic depicting Yeast Version 1 is shown in FIG. 2. A yeast strain constitutively expresses two different proteins (A and B). The proteins are chromogenic or fluorescent (herein referred to as chromogenic protein A or B), but share the characteristic that they are visible by eye when expressed in yeast. This strain will also carry episomal plasmids that constitutively express a gRNA to either chromogenic protein A or B (gRNA A or gRNA B). The plasmid expressing gRNA A will carry the URA3 auxotrophic marker, whereas the plasmid expressing gRNA B will carry the LYS2 auxotrophic marker. A third plasmid or integrated expression cassette will express Cas9 under the control of a galactose-inducible promoter. In the absence of galactose, no gene editing events shall take place and the yeast will be the color resulting from coexpression of chromogenic protein A and B. In the presence of galactose, Cas9 expression is induced and gRNA A or B will be incorporated into Cas9, leading to targeting of chromogenic gene A or B. In the event that both chromogenic gene A and B are targeted, the yeast will no longer express chromogenic protein A or B and the yeast colonies will appear white in color.

Plating yeast on galactose and 5-fluoro-orotic acid (5-FOA) will both induce Cas9 expression and counterselect for yeast carrying the plasmid expressing gRNA A, which carries the URA3 auxotrophic marker. As a result, only gRNA B will be expressed and incorporated into Cas9, leading to targeting of chromogenic gene B. In the event that chromogenic gene B is targeted and disrupted, then the yeast will no longer express chromogenic protein B and the yeast will appear as the color of chromogenic protein A. Plating yeast on galactose and alpha adipic acid (alpha AA) will both induce Cas9 expression and counterselect for yeast carrying the plasmid expressing gRNA B, which carries the LYS2 auxotrophic marker. As a result, only gRNA A will be expressed and incorporated into Cas9, leading to targeting of chromogenic gene A. In the event that chromogenic gene A is targeted and disrupted, then the yeast will no longer express chromogenic protein A and the yeast will appear as the color of chromogenic protein B.

### Example 3

### Yeast Version 2

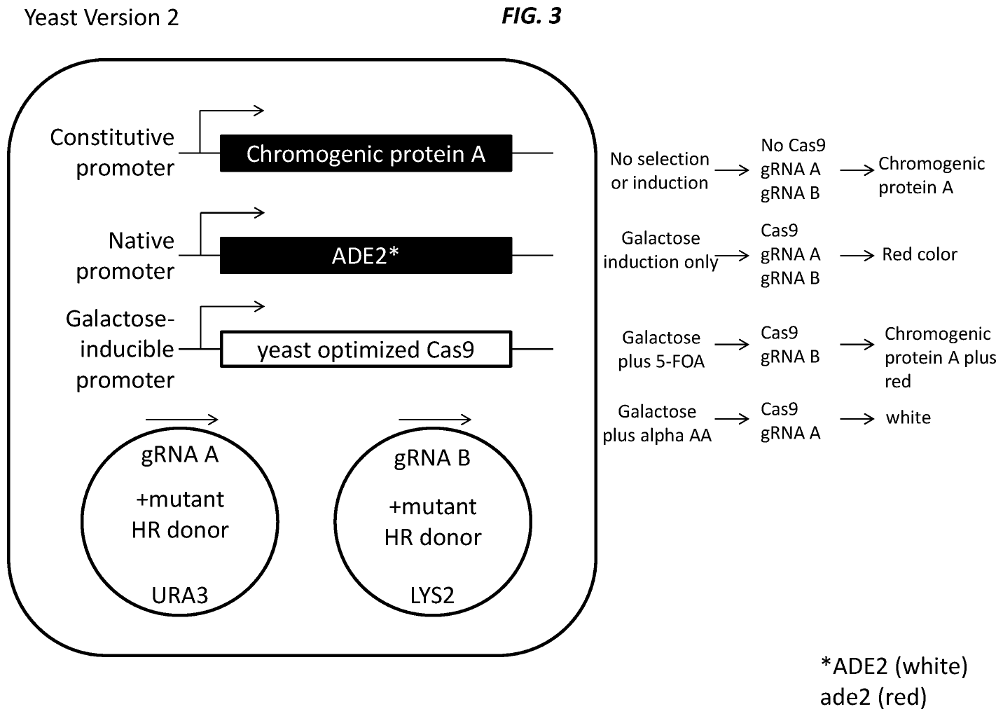

A schematic depicting Yeast Version 2 is shown in FIG. 3. A yeast strain constitutively expresses a chromogenic protein (A). This strain will also carry episomal plasmids that constitutively express a gRNA to either chromogenic protein A (gRNA A) or the ADE2 gene (gRNA B). The plasmid expressing gRNA A will carry the URA3 auxotrophic marker, whereas the plasmid expressing gRNA B will carry the LYS2 auxotrophic marker. A third plasmid or integrated expression cassette will express Cas9 under the control of a galactose-inducible promoter.

In the absence of galactose, no gene editing events shall take place and the yeast will be the color resulting from expression of chromogenic protein A. In the presence of galactose, Cas9 expression is induced and gRNA A or B will be incorporated into Cas9, leading to targeting of chromogenic gene A or the ADE2 gene. In the event that both chromogenic gene A and the ADE2 gene are targeted, the yeast will no longer express chromogenic protein A or ADE2 and the yeast colonies will appear red in color. Plating yeast on galactose and 5-fluoro-orotic acid (5-FOA) will both induce Cas9 expression and counterselect for yeast carrying the plasmid expressing gRNA A, which carries the URA3 auxotrophic marker. As a result, only gRNA B will be expressed and incorporated into Cas9, leading to targeting of the ADE2 gene. In the event that the ADE2 gene is targeted and disrupted, then the yeast will no longer express ADE2 and the yeast will appear as the color of the combination of chromogenic protein A and ade2 yeast (chromogenic protein A+red). Plating yeast on galactose and alpha adipic acid (alpha AA) will both induce Cas9 expression and counterselect for yeast carrying the plasmid expressing gRNA B, which carries the LYS2 auxotrophic marker. As a result, only gRNA A will be expressed and incorporated into Cas9, leading to targeting of chromogenic gene A. In the event that chromogenic gene A is targeted and disrupted, then the yeast will no longer express chromogenic protein A and the yeast will appear white.

### Example 4

### Yeast Version 3

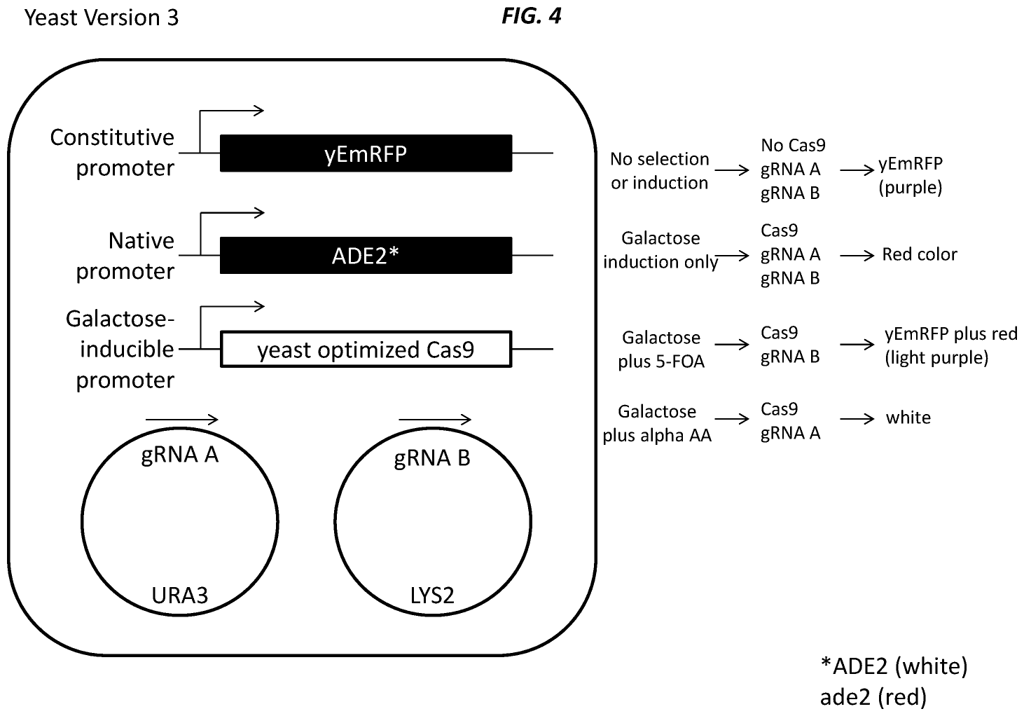

A schematic depicting Yeast Version 3 is shown in FIG. 4. A yeast strain constitutively expresses a yeast-enhanced red fluorescent protein (yEmRFP, Keppler-Ross et al 2008 Genetics). This strain will also carry episomal plasmids that constitutively express a gRNA to either the yEmRFP or the ADE2 gene (gRNA A or gRNA B, respectively). The plasmid expressing gRNA A will carry the URA3 auxotrophic marker, whereas the plasmid expressing gRNA B will carry the LYS2 auxotrophic marker. A third plasmid or integrated expression cassette will express Cas9 under the control of a galactose-inducible promoter.

In the absence of galactose, no gene editing events shall take place and the yeast will be the color resulting from expression of yEmRFP (purple, see Keppler-Ross et al, 2008). In the presence of galactose, Cas9 expression is induced and gRNA A or B will be incorporated into Cas9, leading to targeting of yEmRFP or the ADE2 gene. In the event that both yEmRFP and the ADE2 gene are targeted, the yeast will no longer express yEmRFP or ADE2 and the yeast colonies will appear red in color. Plating yeast on galactose and 5-fluoro-orotic acid (5-FOA) will both induce Cas9 expression and counterselect for yeast carrying the plasmid expressing gRNA A, which carries the URA3 auxotrophic marker. As a result, only gRNA B will be expressed and incorporated into Cas9, leading to targeting of the ADE2 gene.

### Example 5

### Yeast Version 4

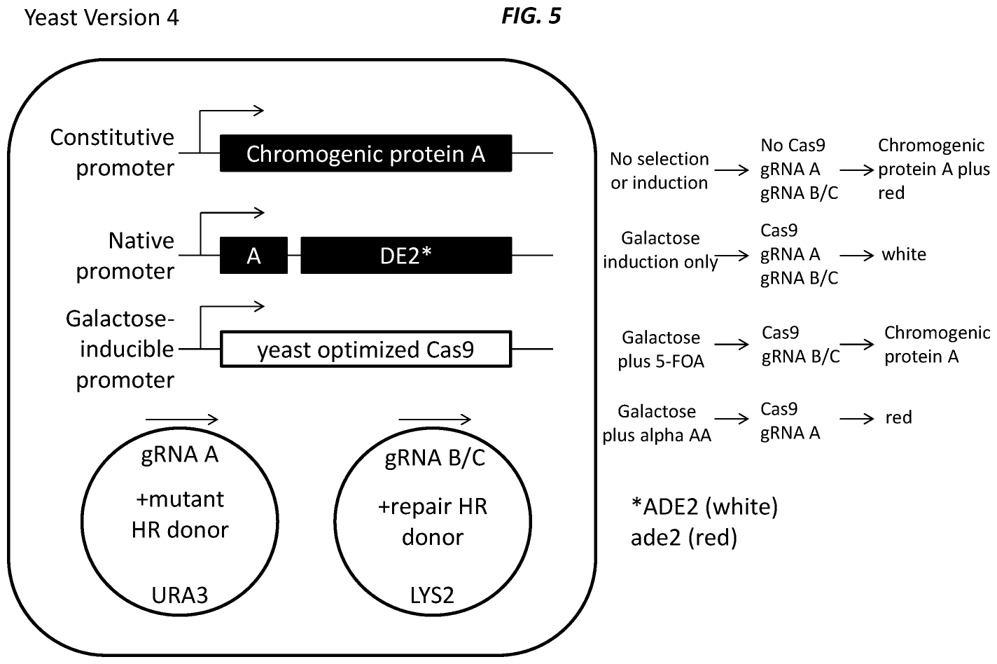

A schematic depicting Yeast Version 4 is shown in FIG. 5. A yeast strain constitutively expresses a yeast-enhanced red fluorescent protein (yEmRFP, Keppler-Ross et al 2008 Genetics). The yeast strain will have an ADE2 gene containing an engineered disruption, similar to an intron, that causes a frameshift and prevents expression of ADE2. This strain will also carry episomal plasmids that constitutively express a gRNA to either the yEmRFP or two gRNAs specific to the 5′ and 3′ ends of the engineered disruption in the ADE2 gene (gRNA A or gRNA B/C, respectively). The plasmid expressing gRNA A will carry the URA3 auxotrophic marker, whereas the plasmid expressing gRNA B/C will carry the LYS2 auxotrophic marker. A third plasmid or integrated expression cassette will express Cas9 under the control of a galactose-inducible promoter.

In the absence of galactose, no gene editing events shall take place and the yeast will be the color resulting from expression of yEmRFP and lack of ADE2 (light purple, see Keppler-Ross et al, 2008, FIG. 1A). In the presence of galactose, Cas9 expression is induced and gRNA A or B will be incorporated into Cas9, leading to targeting of yEmRFP or the ADE2 gene. In the event that both yEmRFP and the ADE2 gene are targeted, the yeast will no longer express yEmRFP or ADE2 and the yeast colonies will appear white in color. Plating yeast on galactose and 5-fluoro-orotic acid (5-FOA) will both induce Cas9 expression and counterselect for yeast carrying the plasmid expressing gRNA A, which carries the URA3 auxotrophic marker. As a result, only gRNA B/C will be expressed and incorporated into Cas9, leading to targeting of the disrupted ADE2 gene. In the event that the disrupted ADE2 gene is targeted and corrected, then the yeast will express ADE2 and the yeast will appear as the color of the combination of yEmRFP and ADE2 yeast (purple). Plating yeast on galactose and alpha adipic acid (alpha AA) will both induce Cas9 expression and counterselect for yeast carrying the plasmid expressing gRNA B/C, which carries the LYS2 auxotrophic marker. As a result, only gRNA A will be expressed and incorporated into Cas9, leading to targeting of yEmRFP. In the event that yEmRFP is targeted and disrupted, then the yeast will no longer express yEmRFP and the yeast will appear red.

### Example 6

### Bacteria Version 1

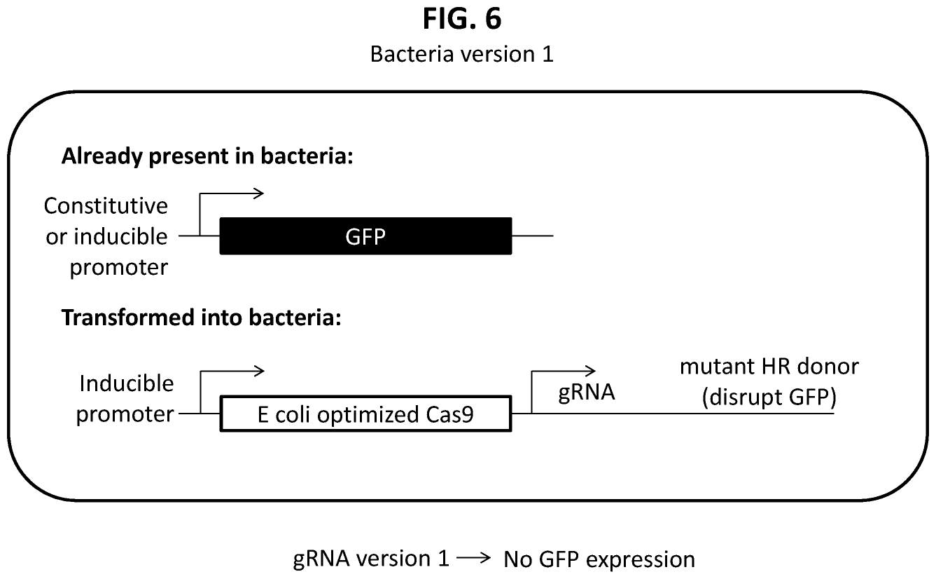

An exemplary schematic depicting Bacteria Version 1 is shown in FIG. 6. Bacteria express (constitutively or inducibly) a protein. The protein is chromogenic or fluorescent (such as GFP, as depicted in FIG. 6), and may be visible by eye when expressed in bacteria. An episomal plasmid that expresses a gRNA to the protein (e.g., GFP) can be transformed into the bacteria. The plasmid expressing gRNA carries the Kanamycin antibiotic resistance marker and also expresses Cas9 under the control of an inducible promoter. In the absence of an inducer, no gene editing events take place and the bacteria will be the color resulting from expression of the chromogenic protein (e.g., GFP). In the presence of an inducer, Cas9 expression is induced and the gRNA will be incorporated into Cas9, leading to targeting of GFP. Repair of the Cas9 cleavage is directed by the HR donor DNA contained in the transformed plasmid, allowing incorporation of specific mutations that prevent GFP expression. In the event that GFP is targeted, the bacteria will no longer express GFP and the bacterial colonies will appear white or beige in color.

### Example 7

### Bacteria Version 2

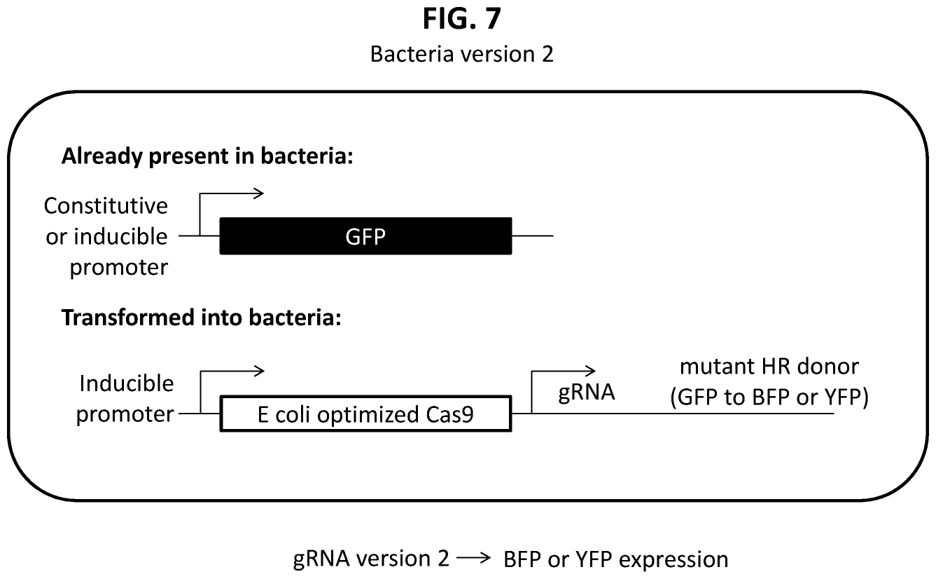

An exemplary schematic depicting Bacteria Version 2 is shown in FIG. 7. Bacteria express (constitutively or inducibly) a protein. The protein is chromogenic or fluorescent (such as GFP, as depicted in FIG. 7), and may be visible by eye when expressed in bacteria. An episomal plasmid that expresses a gRNA to GFP can be transformed into the bacteria. The plasmid expressing gRNA carries an antibiotic resistance marker and also expresses Cas9 under the control of an inducible promoter. In the absence of an inducer, no gene editing events take place and the bacteria will be the color resulting from expression of the chromogenic protein (e.g., GFP). In the presence of an inducer, Cas9 expression is induced and the gRNA will be incorporated into Cas9, leading to targeting of GFP. Repair of the Cas9 cleavage is directed by the HR donor DNA contained in the transformed plasmid, allowing incorporation of specific mutations that prevent GFP expression. In the event that GFP is targeted, the bacteria will express BFP or YFP and the bacterial colonies will appear blue or yellow in color, respectively.

### Example 8

### Bacteria Version 3

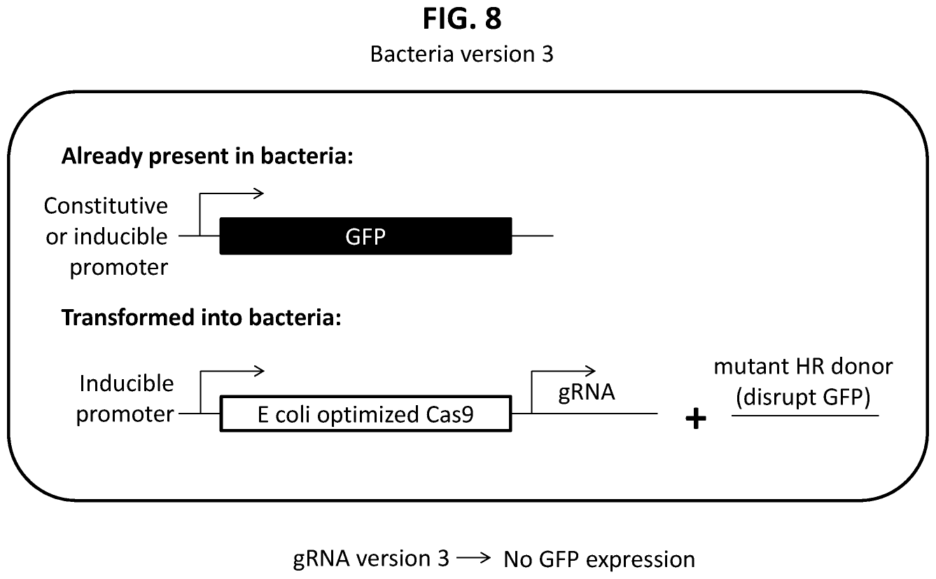

An exemplary schematic depicting Bacteria Version 3 is shown in FIG. 8. Bacteria expresses (constitutively or inducibly) a protein. The protein is chromogenic or fluorescent (such as GFP, as depicted in FIG. 8), and may be visible by eye when expressed in bacteria. An episomal plasmid that expresses a gRNA to GFP can be transformed into the bacteria. The plasmid expressing gRNA carries an antibiotic resistance marker and also expresses

Cas9 under the control of an inducible promoter. In the absence of an inducer, no gene editing events shall take place and the bacteria will be the color resulting from expression of the chromogenic protein (e.g., GFP). In the presence of an inducer, Cas9 expression is induced and the gRNA will be incorporated into Cas9, leading to targeting of GFP. Repair of the Cas9 cleavage is directed by the ssDNA HR donor co-transformed with the plasmid, allowing incorporation of specific mutations that GFP to BFP or YFP. In the event that GFP is targeted, the bacteria will no longer express GFP and the bacterial colonies will appear white or beige in color.

### Example 9

### Bacteria Version 4

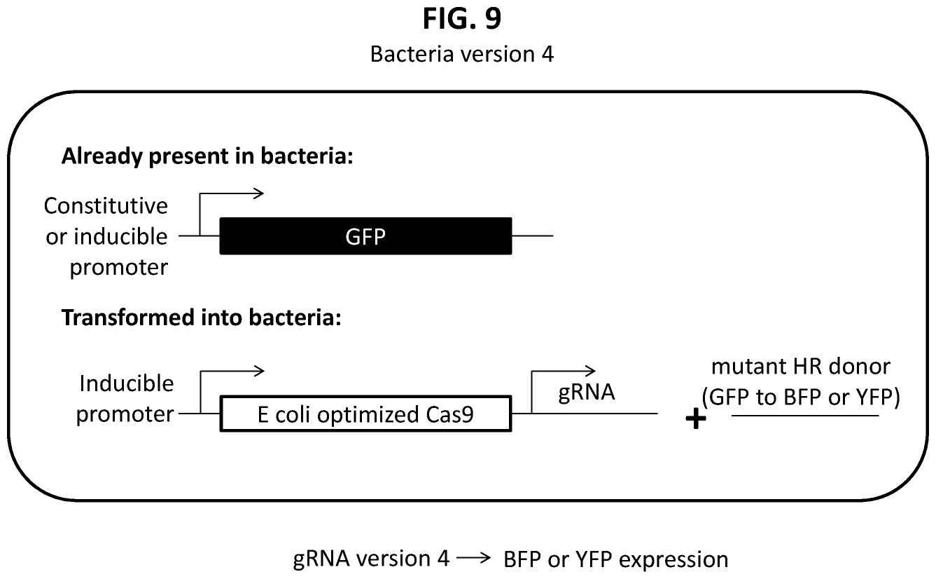

An exemplary schematic depicting Bacteria Version 4 is shown in FIG. 9. Bacteria express (constitutively or inducibly) a protein. The protein is chromogenic or fluorescent (such as GFP, as depicted in FIG. 9), and may be visible by eye when expressed in bacteria. An episomal plasmid that expresses a gRNA to GFP can be transformed into the bacteria. The plasmid expressing gRNA carries an antibiotic resistance marker and also expresses Cas9 under the control of an inducible promoter. In the absence of an inducer, no gene editing events shall take place and the bacteria will be the color resulting from expression of the chromogenic protein (e.g., GFP). In the presence of an inducer, Cas9 expression is induced and the gRNA will be incorporated into Cas9, leading to targeting of GFP. Repair of the Cas9 cleavage is directed by the ssDNA HR donor co-transformed with the plasmid, allowing incorporation of specific mutations that convert GFP to BFP or YFP. In the event that GFP is targeted, the bacteria will express BFP or YFP and the bacterial colonies will appear blue or yellow in color, respectively.

### Example 10

### Bacteria Version 5

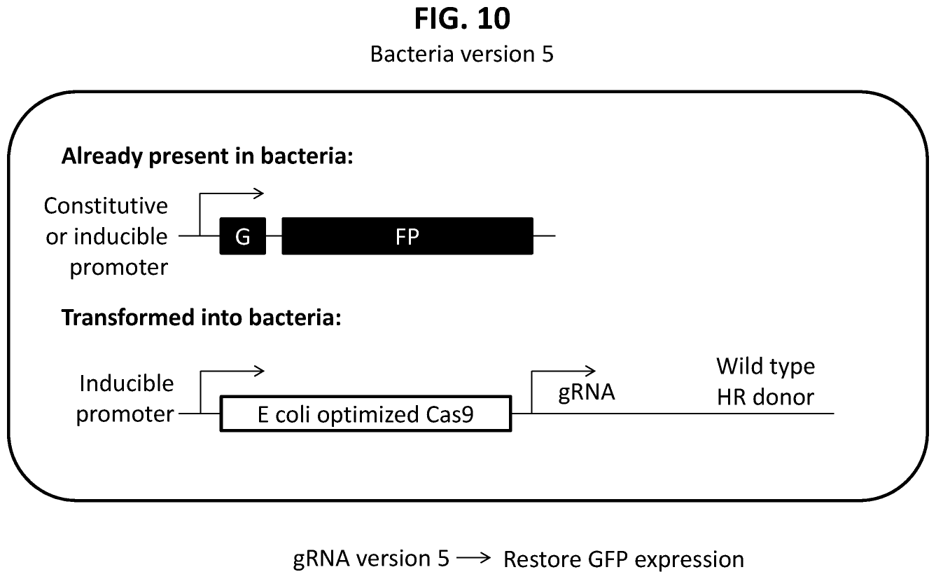

An exemplary schematic depicting Bacteria Version 5 is shown in FIG. 10. Bacteria express a non-functional chromogenic or fluorescent (such as GFP) protein under the control of a constitutive or inducible promoter. An episomal plasmid that expresses a gRNA to GFP can be transformed into the bacteria. The plasmid expressing gRNA also expresses Cas9 under the control of an inducible promoter; if the GFP protein is under the control of an inducible promoter, then the inducible promoter for the GFP and the inducible promoter for Cas9 do not rely on the same method of induction (e.g., do not use the same inducing agent). In the absence of an inducer for the Cas9 promoter, no gene editing events shall take place and the bacteria are the color white. In the presence of an inducer for the Cas9 promoter, Cas9 expression is induced and the gRNA will be incorporated into Cas9, leading to targeting of GFP. Repair of the Cas9 cleavage is directed by the HR donor contained in the transformed plasmid, allowing incorporation of specific mutations that restore GFP expression. In the event that GFP is targeted, the bacteria will express a functional GFP and the bacterial colonies will appear green in color.

### Example 11

### Bacteria Version 6

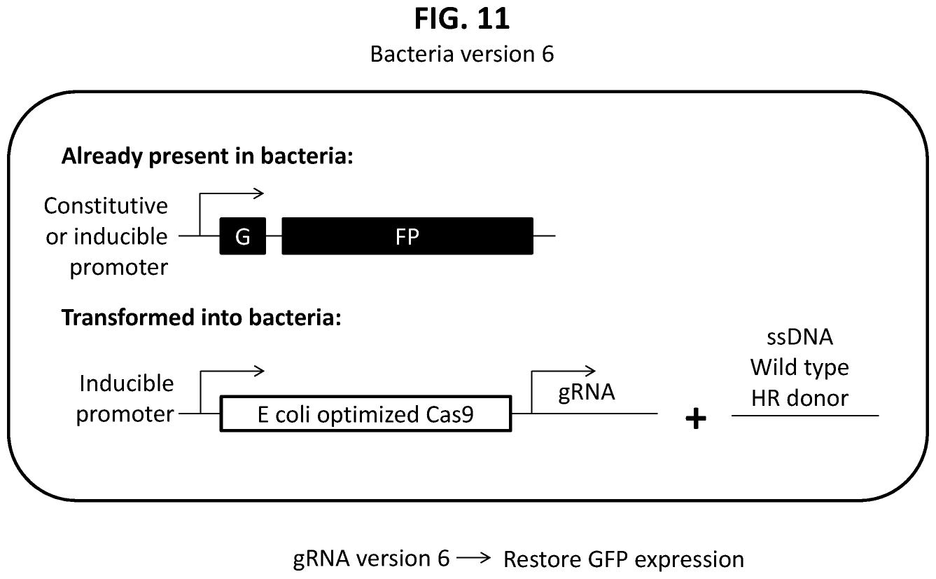

An exemplary schematic depicting Bacteria Version 6 is shown in FIG. 11. Bacteria express a non-functional chromogenic or fluorescent (such as GFP) protein under the control of a constitutive or inducible promoter. An episomal plasmid that expresses a gRNA to GFP can be transformed into the bacteria. The plasmid expressing gRNA also expresses Cas9 under the control of an inducible promoter; if the GFP protein is under the control of an inducible promoter, then the inducible promoter for the GFP and the inducible promoter for Cas9 do not rely on the same method of induction (e.g., do not use the same inducing agent). In the absence of an inducer for the Cas9 promoter, no gene editing events shall take place and the bacteria are the color white. In the presence of an inducer for the Cas9 promoter, Cas9 expression is induced and the gRNA will be incorporated into Cas9, leading to targeting of GFP. Repair of the Cas9 cleavage is directed by the ssDNA HR donor co-transformed with the plasmid, allowing incorporation of specific mutations that restore GFP expression. In the event that GFP is targeted, the bacteria will express a functional GFP and the bacterial colonies will appear green in color.

### Example 12

### Bacteria Version 7

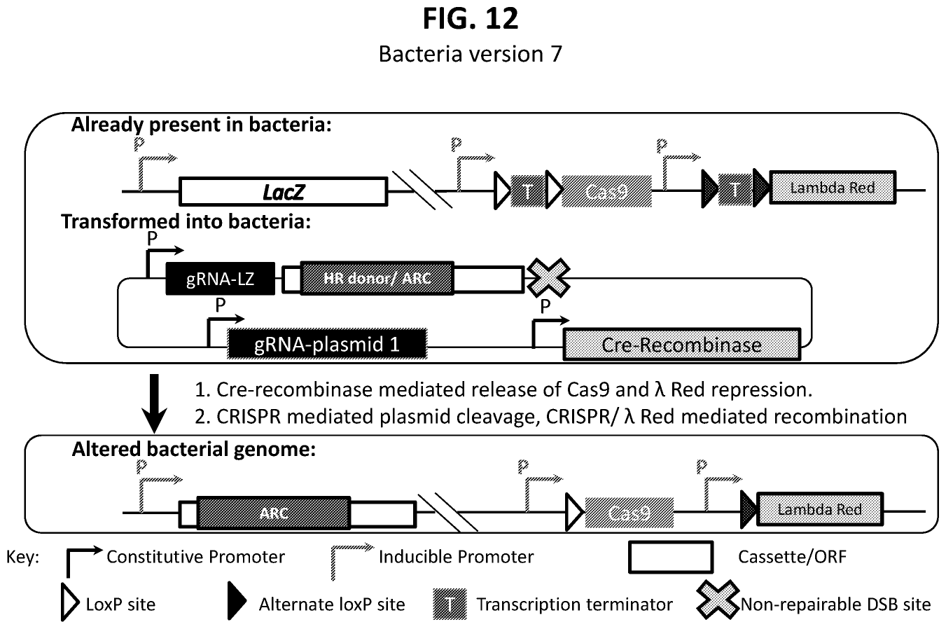

An exemplary schematic depicting Bacteria Version 7 is shown in FIG. 12. Bacteria have been genetically altered to have inducible Cas9 and inducible lambda red expression cassettes inserted within the bacterial genome. Both of these inducible promoters have loxP or loxP variant sequences that flank a transcription terminator sequence directly following the promoter. These gene regulatory elements will block transcription, preventing expression of the induced proteins.

These genetically altered bacteria are transformed with a single plasmid that contains an antibiotic resistance cassette (ARC) lacking its own promoter that is flanked on the 5′ and 3′ ends with short portions of an endogenous target gene (LacZ in FIG. 12), a gRNA that recognizes the targeted gene (“gRNA-LZ” in FIG. 12), a constitutively expressed Cre-recombinase cassette, and a “self-destruct” gRNA that targets a self region within the plasmid, termed a non-repairable DSB site (“gRNA-plasmid 1” in FIG. 12). Upon expression of Cre recombinase, both of the transcription termination sequences will be excised, leaving a single loxP or loxP variant “scar” behind, and transcription and translation will become undeterred. A constitutively active Cas9 cassette will initiate Cas9 expression. The plasmid will be destroyed, and only bacteria that integrate the ARC will survive selection by that antibiotic. By design, there is a heightened risk for off-target insertion within the bacterial genome other than targeted gene. Upon induction of CRISPR-Cas and lambda red, recombination repair will allow the cell to express the antibiotic resistance protein and lose normal targeted gene expression (LacZ in FIG. 12). Bacteria are screened for loss of the normal targeted gene's function and the ability to survive in the presence of the antibiotic.

### Example 13

### Bacteria Version 8

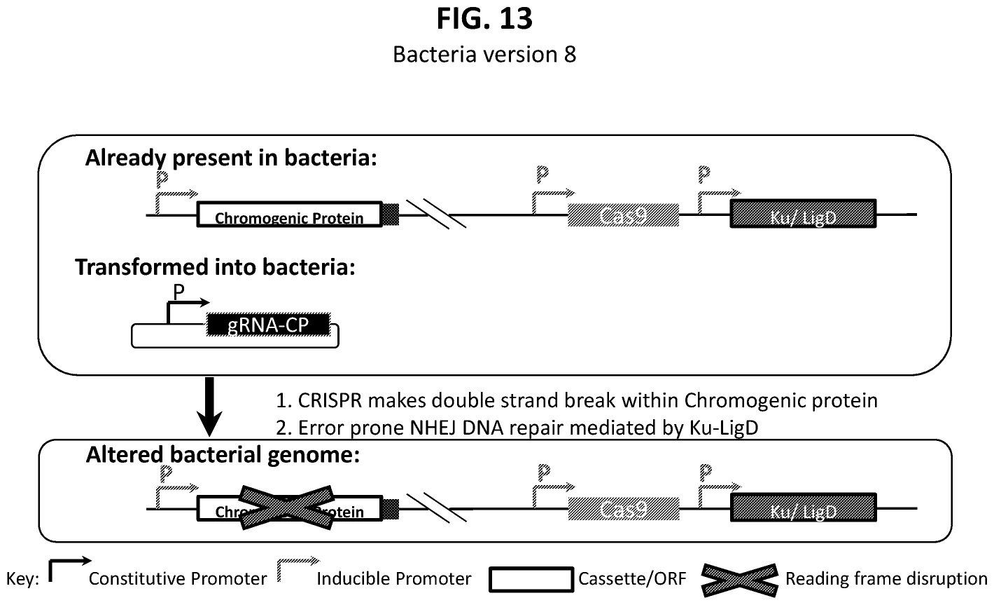

An exemplary schematic depicting Bacteria Version 8 is shown in FIG. 13. Bacteria have been genetically altered to have inducible Cas9 and Ku and LigD expression cassettes present. Additionally, an endogenous gene is replaced with a chromogenic protein ORF. These altered bacteria will express the chromogenic protein upon activation of the endogenous gene.

The genetically altered bacteria are transformed with a single plasmid that has a cassette driving expression of an sgRNA that recognizes the chromogenic protein. Upon induction, a DSB will occur within the chromogenic protein. In contrast to unaltered E coli, the altered bacteria are able to perform non-homologous end joining (NHEJ) based repair of the DSB break. Ku and LigD proteins will recognize the DSB and rejoin the two broken ends. Because this method of DSB repair is prone to error, a significant percentage of E. coli will lose the ability to express the chromogenic protein. A user can quantify the percentage of bacteria that lose the expression of the chromogenic protein, as compared to the percentage that retain expression.

### Example 14

### Bacteria Version 9

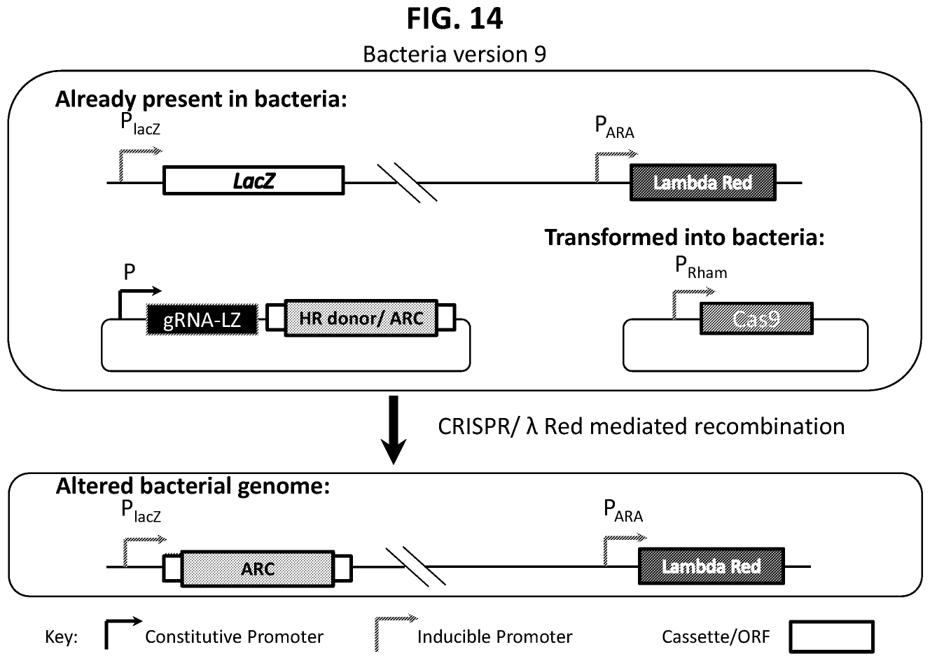

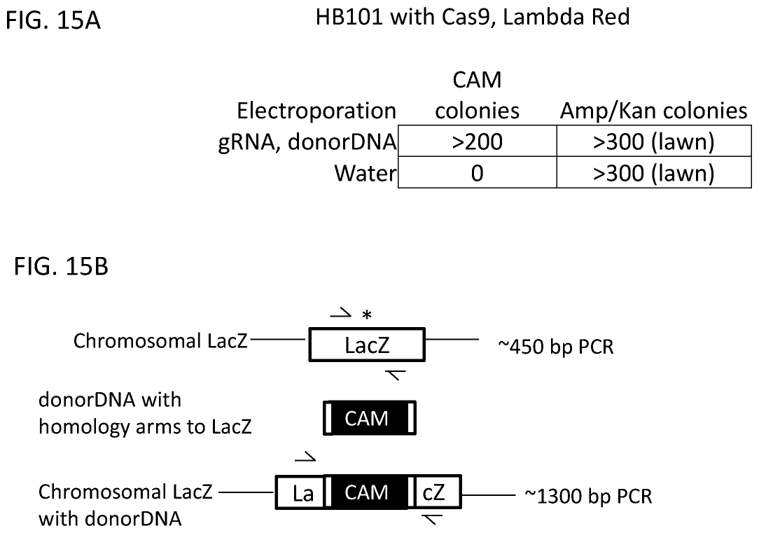

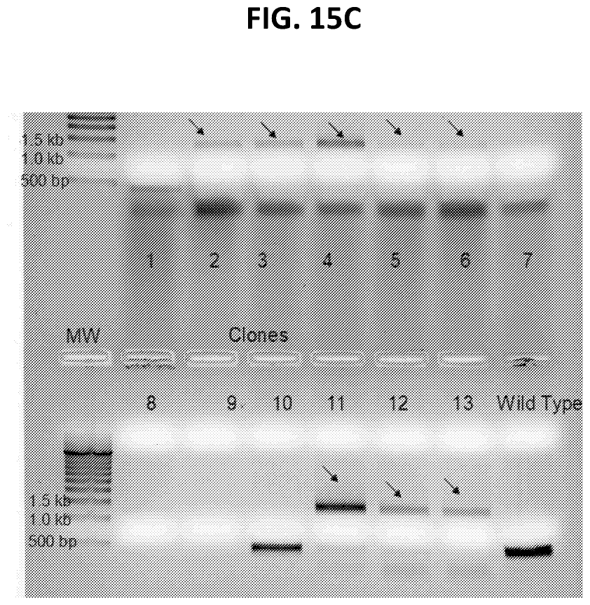

An exemplary schematic depicting Bacteria Version 9, a two-plasmid system for simultaneous loss and gain of function, is shown in FIG. 14. Bacteria are genetically altered to have inducible CRISPR activity and inducible lambda red expression cassettes. As an example, unaltered HB101 strain or HME63 strain E. coil express β-galactosidase under control of the native or modified lacZ promoter, which is induced by the presence of Isopropyl β-D-1-thiogalactopyranoside (IPTG). In HME63 strain bacteria, an arabinose inducible lambda red cassette is located within the bacteria's chromosome. For HB101 strain bacteria the arabinose inducible lambda red cassette is located in an additional plasmid already present in the bacteria. The bacteria, e.g., one of the HME63 or HB101 bacterial strains, are transformed with two unique plasmids. The first plasmid contains a constitutively expressing gRNA that recognizes the lacZ gene and an antibiotic resistance cassette (ARC) lacking its own promoter that is flanked on the 5′ and 3′ ends with short portions of the lacZ gene. The ARC is in the same reading frame as β-galactosidase. The second plasmid contains a rhamnose-inducible promoter driving Cas9 nuclease. In the presence of all four of the described elements, a CRISPR-Cas mediated double strand break (DSB) will occur within the genome at the lacZ gene. Lambda red proteins will recognize the DSB and initiate homologous recombination based repair utilizing the lacZ flanked ARC. E. coli are screened for loss of blue color from X-gal stain and the ability to survive in the presence of the novel antibiotic. Only upon successful CRISPR-Cas initiated recombination repair will the modified E. coli be able to both express the antibiotic resistance protein and lose normal β-galactosidase expression.

### Example 15

### Cas9-Mediated Targeting of Genomic Locus

E. coli HB101 bacteria were transformed with a plasmid containing a Cas9 endonuclease and a plasmid containing the Lambda red recombinase genes. The cells were subsequently transformed with single-stranded gRNA targeting a genomic target (LacZ) and double-stranded donor DNA containing a chloramphenicol (CAM) gene flanked by LacZ homology arms. Cells were plated on CAM plates and Ampicillin/Kanamycin plates. Cells that underwent homologous recombination acquired resistance to CAM were able to grow on CAM plates. CAM-resistant colonies were screened by PCR. The PCR product for wild type LacZ is 450 bp, whereas the PCR product for LacZ after Cas9-mediated recombination of the donor DNA is 1300 bp. Of the 10 colonies producing a PCR result, 8 were positive for the Cas9-mediated recombination of the donor DNA.

### Example 16

### Cas9-Mediated Repair of Genomic Locus

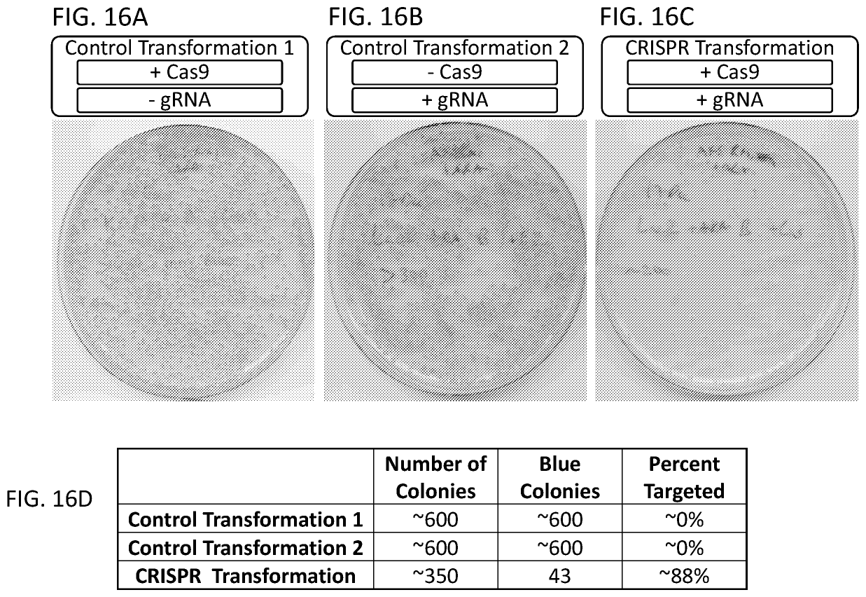

A LacZ DNA donor plasmid was created to insert a stop codon into the β-galactosidase reading frame, thereby creating a 163 amino acid truncated non-functioning enzyme. HB101 bacteria containing a lambda red expressing plasmid and the LacZ donor plasmid were transformed with plasmids containing the following expression cassettes: a Cas9 cassette but no gRNA expressing cassette (FIG. 16A, Control Transformation 1), a gRNA expressing cassette but no Cas9 expressing cassette (FIG. 16B, Control Transformation 2), or a gRNA expressing cassette and a Cas9 expressing cassette (FIG. 16C, CRISPR Transformation). All bacteria were grown in the presence of ampicillin, kanamycin, streptomycin, rhamnose, arabinose, X-gal substrate, and IPTG. IPTG induces the LacZ promoter to drive expression of β-galactosidase, which converts the colorless X-gal substrate into a blue product. Only bacteria with intact β-galactosidase will become blue while cells that lack functional β-galactosidase will remain white. 100% of cells that did not have CRISPR activity showed normal β-galactosidase. When CRISPR was activated, approximately 88% of 350 colonies were white, displaying a lack of β-galactosidase activity, while 43 of the ˜350 colonies seen were blue suggesting the presence of functional β-galactosidase (FIG. 16D).

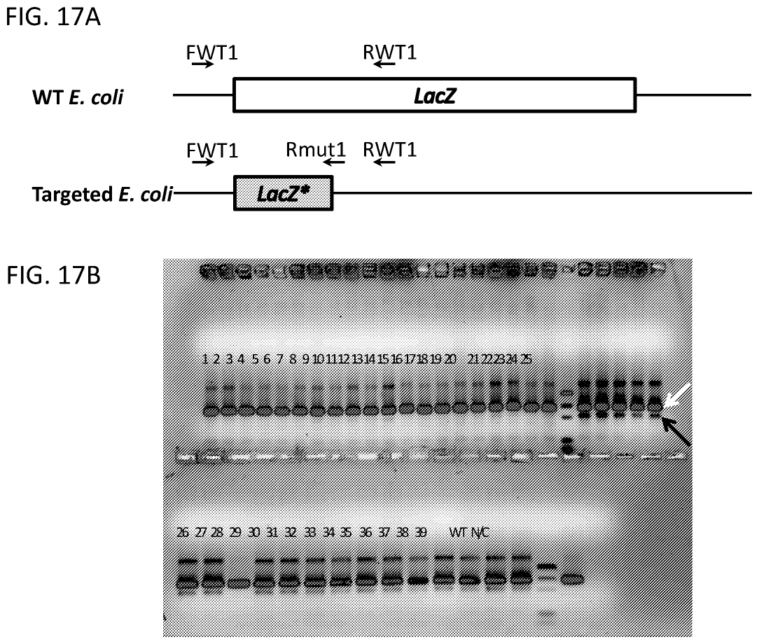

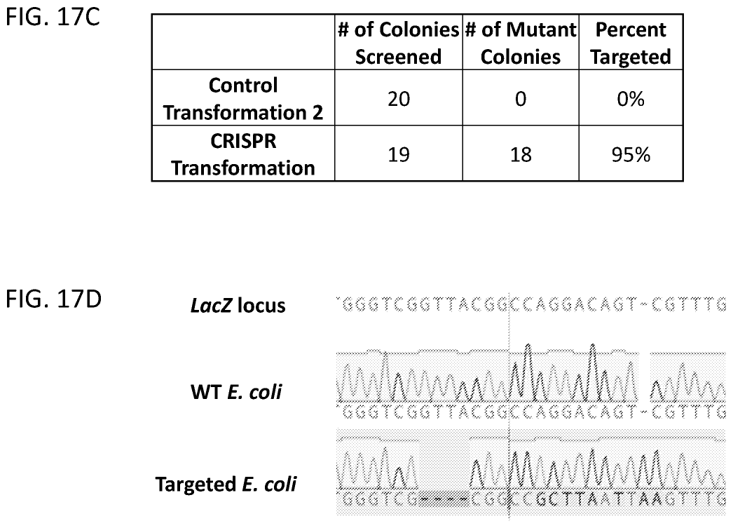

In order to further verify genomic targeting of the LacZ locus, polymerase chain reaction based genotyping was performed. Two oligonucleotides (FWT1 and FWT2) produce a 686 base pair amplicon in wild-type and targeted bacteria. Correctly targeted LacZ mutant E. coli produce a 550 base pair amplicon from FWT1 and Rmut1 oligonucleotides (FIG. 17A). 0 of 20 colonies from control transformation 2 showed a 550 base pair mutant PCR product. 18 of 19 colonies from CRISPR transformations produced both a wild-type band and the mutant band (white and black arrow, respectively, FIG. 17B-C). Sanger sequencing was performed on the PCR product of several positive mutants to confirm the expected insertion of the premature stop codon. All showed identical sequence matching the donor plasmid (representative sequence trace, FIG. 17D).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

INFORMAL SEQUENCE LISTING

SEQ ID NO: 1-Yeast codon optimized Cas9
ATGTATCCATATGATGTTCCAGATTACGCTCCACCTAA
GAAGAAACGTAAGGTTGACAAAAAGTACTCCATCGGT
TTAGATATTGGTACCAACTCTGTCGGTTGGGCCGTTAT
TACTGATGAATACAAGGTTCCATCTAAGAAGTTCAAA
GTTTTAGGTAACACTGATAGACACTCCATTAAGAAGAA
TTTGATCGGTGCTTTGTTGTTCGATTCCGGTGAAACC
GCCGAAGCTACCAGATTAAAGAGAACCGCTAGAAGAAG
ATATACCAGAAGAAAGAACAGAATTTGTTACTTGCAA
GAAATTTTCTCCAACGAAATGGCCAAGGTTGATGATTC
TTTCTTTCACAGATTGGAAGAATCCTTTTTAGTCGAA
GAAGATAAGAAACACGAAAGACACCCAATCTTCGGTAA
CATTGTCGACGAAGTCGCTTATCACGAAAAATATCCA
ACTATTTACCACTTGAGAAAGAAGTTAGTCGACTCCAC
CTACAAAGCTGACTTGAGATTGATTTATTTGGCTTTG
GCTCACATGATTAAGTTCAGAGGTCACTTCTTAATCGA
AGGTGACTTGAACCCTGACAATTCCGATGTTGACAAG
TTGTTCATCCAATTGGTCCAAACCTATAATCAATTGTT
CGAAGAAAATCCAATCAACGCTTCCGGTGTTGACGCT
AAAGCCATCTTGTCTGCCAGATTGTCCAAGTCCCGTCG
TTTAGAAAACTTGATTGCTCAATTGCCAGGTGAAAAG
AAGAACGGTTTGTTTGGTAACTTGATTGCTTTGTCCTT
GGGTTTAACCCCAAACTTCAAGTCTAACTTCGATTTG
GCCGAAGATGCTAAGTTGCAATTGTCCAAGGATACTTA
CGATGACGATTTGGATAACTTATTGGCCCAAATCGGT
GACCAATACGCTGACTTGTTCTTGGCCGCTAAGAACTT
ATCCGACGCCATCTTGTTGTCCGACATTTTAAGAGTT
AACACTGAAATTACCAAGGCCCCATTGTCCGCCTCCAT
GATCAAGAGATACGACGAACACCACCAAGACTTGACC
TTATTGAAGGCTTTAGTTCGTCAACAATTACCAGAAAA
GTATAAAGAAATCTTCTTTGATCAATCTAAAAACGGT
TACGCTGGTTATATTGATGGTGGTGCCTCTCAAGAAGA
ATTCTACAAATTTATCAAGCCTATCTTAGAAAAAATG
GACGGTACCGAAGAATTATTGGTCAAGTTAAACAGAGA
AGATTTGTTGCGTAAGCAACGTACTTTCGACAACGGT
TCCATCACCCATCAAATCCACTTGGGTGAATTGCACGC
TATCTTAAGAAGACAAGAAGATTTCTACCCATTCTTG
AAAGATAATAGAGAAAAAATTGAAAAAATTTTGACTTT
CAGAATTCCTTACTACGTTGGTCCATTAGCCAGAGGT
AACTCTAGATTTGCTTGGATGACTAGAAAGTCCGAAGA
AACTATTACCCCATGGAACTTCGAAGAAGTCGTTGAC
AAGGGTGCTTCCGCTCAATCCTTCATTGAAAGAATGAC
CAATTTCGATAAAAACTTACCAAACGAAAAGGTTTTG
CCAAAGCACTCTTTGTTATATGAATACTTCACCGTTTA
CAACGAATTGACTAAAGTCAAGTACGTTACCGAAGGT
ATGAGAAAGCCAGCTTTCTTGTCTGGTGAGCAAAAGAA
GGCTATTGTTGACTTATTATTCAAAACTAACAGAAAG
GTCACTGTCAAGCAATTGAAGGAAGATTATTTCAAAAA
GATCGAATGCTTCGACTCTGTTGAAATCTCTGGTGTT
GAAGATAGATTCAACGCTTCCTTGGGTACCTATCACGA
TTTGTTGAAAATCATCAAGGACAAGGACTTTTTGGAT
AACGAGGAAAACGAAGACATTTTAGAAGATATTGTTTT
GACTTTGACCTTGTTCGAAGACAGAGAAATGATCGAA
GAAAGATTGAAGACCTACGCTCATTTGTTCGACGATAA
AGTCATGAAACAATTGAAGAGAAGAAGATATACCGGT
TGGGGTAGATTATCTAGAAAGTTAATTAATGGTATTAG
AGACAAGCAATCCGGTAAGACCATCTTGGATTTCTTA
AAATCTGACGGTTTCGCTAACCGTAACTTCATGCAATT
GATTCATGATGACTCTTTGACCTTCAAAGAAGATATT
CAAAAGGCTCAAGTCTCTGGTCAAGGTGATTCTTTGCA
CGAACATATTGCTAACTTGGCTGGTTCCCCTGCCATT
AAGAAGGGTATTTTGCAAACTGTTAAGGTCGTTGACGA
ATTGGTCAAGGTTATGGGTAGACACAAACCAGAAAAC
ATCGTCATTGAAATGGCCAGAGAAAACCAAACCACCCA
AAAAGGTCAAAAGAACTCCCGTGAAAGAATGAAGAGA
ATCGAAGAAGGTATCAAGGAGTTGGGTTCTCAAATTTT
AAAGGAACATCCAGTCGAGAACACTCAATTGCAAAAC
GAAAAGTTGTACTTGTACTACTTACAAAACGGTAGAGA
TATGTACGTCGATCAAGAATTAGACATTAATAGATTG
TCTGACTACGACGTCGACCATATTGTCCCACAATCTTT
CTTAAAGGACGATTCCATTGACAACAAAGTTTTAACT
AGATCCGACAAAAACAGAGGTAAGTCTGATAACGTTCC
TTCTGAAGAAGTCGTCAAGAAGATGAAGAACTACTGG
AGACAATTGTTGAACGCCAAATTGATCACCCAAAGAAA
GTTCGACAACTTAACCAAGGCTGAAAGAGGTGGTTTG
TCTGAATTGGATAAGGCTGGTTTTATTAAGAGACAATT
GGTCGAAACCAGACAAATTACTAAACATGTTGCTCAA
ATCTTGGATTCTCGTATGAATACCAAATACGACGAAAA
CGATAAATTGATTAGAGAAGTCAAGGTTATTACTTTG
AAGTCCAAGTTGGTTTCTGATTTCCGTAAGGACTTCCA
ATTCTACAAAGTTAGAGAAATTAACAATTACCACCAC
GCTCATGACGCTTACTTGAACGCTGTCGTTGGTACTGC
CTTGATTAAGAAGTACCCAAAATTGGAATCCGAATTC
GTTTATGGTGACTACAAAGTTTACGATGTCAGAAAAAT
GATTGCTAAGTCTGAACAAGAGATTGGTAAAGCTACT
GCCAAATATTTCTTTTACTCTAACATCATGAACTTCTT
TAAGACCGAAATCACTTTAGCTAACGGTGAAATTCGT
AAGAGACCATTAATCGAAACTAACGGTGAAACTGGTGA
AATTGTCTGGGATAAGGGTAGAGATTTCGCCACCGTT
CGTAAGGTTTTGTCTATGCCTCAAGTTAATATCGTCAA
GAAGACCGAAGTCCAAACCGGTGGTTTTTCTAAGGAA
TCTATCTTGCCAAAGAGAAATTCTGACAAGTTGATTGC
TAGAAAGAAAGACTGGGATCCAAAGAAGTACGGTGGT
TTTGACTCCCCAACTGTTGCTTACTCCGTTTTGGTTGT
TGCTAAGGTTGAAAAGGGTAAGTCTAAGAAGTTAAAG
TCTGTTAAGGAATTGTTGGGTATTACCATTATGGAAAG
ATCTTCTTTTGAGAAAAACCCAATTGACTTTTTAGAG
GCTAAGGGTTACAAGGAAGTTAAGAAGGACTTGATCAT
TAAATTGCCAAAGTATTCTTTGTTCGAATTGGAAAAC
GGTCGTAAGCGTATGTTGGCCTCTGCTGGTGAATTACA
AAAGGGTAACGAATTAGCTTTGCCTTCTAAATACGTT
AACTTTTTATACTTGGCTTCCCATTACGAAAAGTTAAA
AGGTTCTCCAGAAGACAATGAACAAAAACAATTGTTC
GTTGAACAACACAAGCATTACTTGGACGAAATTATTGA
ACAAATTTCTGAATTTTCCAAGAGAGTTATCTTAGCC
GACGCTAACTTGGACAAGGTCTTGTCTGCTTACAATAA
GCATAGAGACAAGCCAATCCGTGAACAAGCCGAAAAC
ATCATTCACTTGTTCACTTTGACTAACTTGGGTGCTCC
AGCTGCCTTCAAGTACTTCGACACCACTATCGACAGA
AAGAGATACACTTCTACTAAGGAGGTTTTAGATGCCAC
CTTGATTCACCAATCTATTACTGGTTTGTACGAGACT
AGAATTGATTTGTCCCAATTAGGTGGTGATCCACCTAA
GAAGAAGAGAAAGGTTTAA
SEQ ID NO: 2-E. coli codon optimized Cas9
ATGTATCCATACGACGTGCCTGACTATGCGGACAAAAAGT
ACTCCATCGGTTTGGACATCGGGACGAATAGCGTTGGGTG
GGCGGTGATTACAGATGAATATAAGGTGCCTAGTAAAAAA
TTCAAAGTATTAGGCAATACCGATCGTCATAGCATTAAGA
AGAACCTGATTGGAGCATTGCTTTTTGATTCGGGTGAAAC
CGCAGAAGCAACGCGTTTGAAGCGCACAGCACGTCGTCGC
TACACACGCCGCAAAAATCGTATTTGTTATCTTCAAGAAA
TTTTTTCAAATGAGATGGCAAAGGTCGACGATTCTTTTTT
CCATCGTTTAGAGGAATCTTTTCTTGTGGAGGAAGATAAG
AAGCACGAGCGTCACCCAATCTTTGGTAATATTGTCGACG
AAGTTGCGTACCATGAAAAGTACCCAACGATCTACCACCT
GCGTAAGAAATTGGTGGACTCGACATACAAGGCGGATCTT
GCGTTGATCTATCTTGCTTTGGCCCACATGATTAAGTTCC
GCGGGCATTTCTTAATTGAAGGAGACTTAAACCCGGATAA
CTCAGATGTTGACAAGCTTTTTATTCAGCTTGTGCAAACT
TACAATCAACTTTTCGAAGAAAACCCTATCAACGCCTCTG
GTGTGGATGCGAAGGCGATCCTTTCGGCGCGCCTGTCAAA
GAGTCGTCGCTTGGAGAATTTGATTGCGCAGTTGCCGGGG
GAGAAGAAAAATGGCCTGTTTGGAAACCTGATTGCGCTTT
CTCTTGGATTAACTCCCAACTTTAAGTCAAACTTCGACTT
GGCTGAGGATGCCAAGTTACAGCTGTCCAAAGATACCTAC
GATGATGATCTTGATAACTTGCTGGCTCAAATCGGTGACC
AATACGCGGATCTTTTCTTGGCCGCGAAGAACTTGTCTGA
CGCAATCCTTTTATCGGACATCCTGCGCGTTAACACAGAG
ATCACTAAAGCCCCTCTGTCTGCATCAATGATTAAACGCT
ACGACGAACACCATCAGGATTTGACATTACTGAAAGCCCT
TGTACGTCAACAACTTCCGGAAAAGTACAAAGAAATCTTC
TTCGATCAGTCTAAAAACGGGTACGCCGGGTATATTGATG
GTGGCGCTTCACAGGAGGAGTTTTACAAATTCATTAAACC
TATTCTGGAGAAAATGGATGGAACGGAAGAGTTGTTGGTG
AAGCTTAATCGTGAAGACCTTTTGCGCAAGCAGCGTACGT
TCGATAACGGGTCAATCACACACCAAATCCACTTGGGCGA
GTTACATGCCAATTCTTCGCCGTCAGGAAGATTTCTACCC
TTTCTTGAAAGATAACCGCGAGAAGATTGAAAGATTTTGA
CTTTTCGTATCCCCTACTACGTGGGTCCTTTAGCCCGTGG
AAACAGTCGCTTCGCCTGGATGACCCGCAAGTCAGAAGAA
ACGATCACCCCCTGGAATTTTGAGGAGGTTGTGGATAAGG
GGGCGTCAGCGCAAAGCTTCATCGAACGCATGACGAACTT
CGATAAGAACTTACCTAATGAGAAAGTGCTGCCAAAACAT
AGTCTTCTTTATGAGTACTTCACTGTTACCAATGAGTTAA
CTAAGGTAAAGTATGTTACAGAAGGGATGCGTAAACCCGC
ATTTTTATCCGGTGAGCAAAAGAAAGCTATCGTGGATTTG
TTATTTAAGACTAACCGCAAAGTAACAGTCAAACAATTAA
AAGAAGACTACTTTAAGAAAATTGAGTGCTTTGACTCAGT
GGAGATCTCTGGTGTCGAGGACCGCTTTAATGCCTCATTG
GGAACTTATCACGACTTACTGAAGATTATTAAAGATAAAG
ACTTTCTTGACAACGAAGAGAACGAAGATATTCTGGAGGA
CATCGTCTTAACACTGACTCTGTTCGAGGATCGCGAGATG
ATTGAGGAACGCTTGAAAACTTATGCCCACTTATTTGATG
ACAAAGTGATGAAACAACTTAAACGTCGTCGCTACACCGG
ATGGGGTCGTTTATCACGTAAATTAATCAACGGCATTCGC
GATAAGCAGTCCGGCAAAACAATCCTTGATTTTCTGAAGT
CCGACGGATTCGCGAATCGCAACTTTATGCAGCTGATCCA
CGATGATAGTCTTACCTTCAAGGAGGATATTCAAAAAGCC
CAGGTATCGGGGCAAGGTGACTCCCTGCACGAACATATTG
CGAATTTGGCCGGGTCTCCGGCAATCAAGAAAGGTATTTT
ACAGACCGTTAAAGTCGTGGATGAATTGGTTAAGGTAATG
GGCCGTCATAAACCGGAGAATATCGTAATTGAAATGGCGC
GTGAGAATCAGACAACTCAGAAAGGACAAAAGAATagcCG
CGAACGTATGAAACGTATTGAAGAGGGAATCAAAGAATTA
GGGAGTCAGATCTTAAAAGAACATCCAGTTGAAAACACCC
AATTGCAAAACGAAAAGTTATACTTATACTACCTTCAGAA
CGGCCGCGATATGTACGTAGATCAGGAATTAGACATTAAC
CGCCTGTCAGATTACGATGTCGACCATATTGTTCCTCAGT
CTTTCTTAAAGGACGATAGCATCGATAATAAAGTTTTAAC
TCGTTCGGATAAAAACCGTGGAAAATCCGACAACGTCCCA
TCTGAGGAGGTAGTCAAGAAGATGAAGAACTACTGGCGTC
AGTTACTGAACGCGAAATTGATCACTCAGCGCAAATTTGA
TAATTTGACTAAAGCCGAACGCGGTGGTTTGTCGGAGTTA
GACAAAGCCGGCTTCATCAAACGCCAGCTTGTAGAGACCC
GCCAGATTACGAAGCACGTTGCCCAGATTCTTGACAGCCG
TATGAACACCAAGTACGATGAGAATGATAAACTGATTCGC
GAGGTGAAGGTAATCACGTTAAAGAGCAAACTGGTAAGTG
ATTTTCGTAAGGACTTTCAATTTTACAAAGTGCGCGAGAT
CAACAACTATCACCATGCGCATGATGCCTATTTGAATGCC
GTAGTCGGTACAGCTTTGATTAAGAAGTATCCTAAGTTGG
AGTCAGAATTtGTCTACGGCGACTACAAGGTGTATGACGT
ACGCAAGATGATTGCGAAGTCCGAGCAGGAAATTGGCAAG
GCCACTGCTAAGTACTTCTTTTATTCTAACATTATGAACT
TCTTCAAAACCGAAATCACCCTTGCGAACGGGGAAATTCG
TAAGCGCCCGTTGATCGAAACAAATGGCGAGACTGGTGAA
ATTGTTTGGGACAAAGGTCGCGATTTTGCAACGGTGCGTA
AAGTATTAAGTATGCCCCAGGTAAATATTGTCAAGAAGAC
GGAAGTGCAAACCGGTGGGTTTTCTAAAGAATCAATTTTG
CCGAAGCGTAATTCTGATAAATTAATTGCGCGCAAAAAGG
ACTGGGACCCTAAGAAATACGGCGGGTTCGACTCACCCAC
GGTCGCCTATTCCGTGTTAGTTGTTGCGAAAGTCGAGAAA
GGTAAAAGCAAGAAACTTAAGTCTGTTAAGGAATTGTTAG
GAATCACGATTATGGAACGTAGCTCATTTGAGAAAAACCC
CATTGACTTTTTGGAGGCGAAAGGTTACAAGGAAGTTAAA
AAAGACCTGATTATTAAACTTCCCAAGTACAGCCTTTTCG
AACTGGAAAATGGTCGTAAGCGCATGCTGGCGTCGGCCGG
TGAACTGCAAAAGGGGAATGAGTTGGCCCTGCCATCGAAG
TATGTGAATTTCTTATACCTGGCGTCCCATTACGAAAAAT
TAAAAGGCTCACCGGAAGATAATGAGCAAAAGCAGCTGTT
TGTGGAGCAACACAAACATTACTTAGATGAAATTATCGAA
CAGATTTCGGAGTTCAGCAAGCGCTGTATTCTTGCGGACG
CGAATCTTGATAAGGTTTTATCTGCTTACAACAAGCACCG
CGACAAGCCGATCCGTGAGCAAGCTGAGAACATTATTCAT
TTATTCACACTGACTAATCTTGGGGCGCCAGCTGCCTTTA
AATACTTTGACACCACCATTGACCGCAAACGCTACACAAG
TACAAAAGAAGTCTTAGACGCGACACTGATTCATCAATCC
ATCACTGGTTTATATGAAACACGCATCGATCTTTCGCAAC
TGGGAGGTGATTAA
SEQ ID NO: 3-S. pyognes Cas9
ATGGATAAGAAATACTCAATAGGCTTAGATATCGGCACAAAT
AGCGTCGGATGGGCGGTGATCACTGATGAATATAAGGTTCC
GTCTAAAAAGTTCAAGGTTCTGGGAAATACAGACCGCCACAG
TATCAAAAAAAATCTTATAGGGGCTCTTTTATTTGACAGTG
GAGAGACAGCGGAAGCGACTCGTCTCAAACGGACAGCTCGTA
GAAGGTATACACGTCGGAAGAATCGTATTTGTTATCTACAG
GAGATTTTTTCAAATGAGATGGCGAAAGTAGATGATAGTTTC
TTTCATCGACTTGAAGAGTCTTTTTTGGTGGAAGAAGACAA
GAAGCATGAACGTCATCCTATTTTTGGAAATATAGTAGATGA
AGTTGCTTATCATGAGAAATATCCAACTATCTATCATCTGC
GAAAAAAATTGGTAGATTCTACTGATAAAGCGGATTTGCGCT
TAATCTATTTGGCCTTAGCGCATATGATTAAGTTTCGTGGT
CATTTTTTGATTGAGGGAGATTTAAATCCTGATAATAGTGAT
GTGGACAAACTATTTATCCAGTTGGTACAAACCTACAATCA
ATTATTTGAAGAAAACCCTATTAACGCAAGTGGAGTAGATGC
TAAGCGATTCTTTCCTGCACGATTGAGTAAATCAAGACGAT
TAGAAAATCTCATTGCTCAGCTCCCCGGTGAGAAGAAAAATG
GCTTATTTGGGAATCTCATTGCTTTGTCATTGGGTTTGACC
CCTAATTTTAAATCAAATTTTGATTTGGCAGAAGATGCTAAA
TTACAGCTTTCAAAAGATACTTACGATCATGATTTAGATAA
TTTATTGGCGCAAATTGGAGATCAATATGCTGATTTGTTTTT
GGCAGCTAAGAATTTATCAGATGCTATTTTACTTTCAGATA
TCCTAAGAGTAAATACTGAAATAACTAAGGCTCCCCTATCAG
CTTCAATGATTAAACGCTACGATGAACATCATCAAGACTTG
ACTCTTTTAAAAGCTTTAGTTCGACAACAACTTCCAGAAAAG
TATAAAGAAATCTTTTTTGATCAATCAAAAAACGGATATGC
AGGTTATATTGATGGGGGAGCTAGCCAAGAAGAATTTTATAA
ATTTATCAAACCAATTTTAGAAAAAATGGATGGTACTGAGG
AATTATTGGTGAAACTAAATCGTGAAGATTTGCTGCGCAAGC
AACGGACCTTTGACAACGGCTCTATTCCCCATCAAATTCAC
TTGGGTGAGCTGCATGCTATTTTGAGAAGACAAGAAGACTTT
TATCCATTTTTAAAAGACAATCGTGAGAAGATTGAAAAAAT
CTTGACTTTTCGAATTCCTTATTATGTTGGTCCATTGGCGCG
TGGCAATAGTCGTTTTGCATGGATGACTCGGAGGTCTGAAG
AAACAATTACCCCATGGAATTTTGAAGAAGTTGTCGATAAAG
GTGCTTCAGCTCAATCATTTATTGAACGCATGACAAACTTT
GATAAAAATCTTCCAAATGAAAAAGTACTACCAAAACATAGT
TTGCTTTATGAGTATTTTACGGTTTATAACGAATTGACAAA
GGTCAAATATGTTACTGAAGGAATGCGAAAACCAGCATTTCT
TTCAGGTGAACAGAAGAAAGCCATTGTTGATTTACTCTTCA
AAACAAATCGAAAAGTAACCGTTAAGCAATTAAAAGAAGATT
ATTTCAAAAAAATAGAATGTTTTGATAGTGTTGAAATTTCA
GGAGTTGAAGATAGATTTAATGCTTCATTAGGTACCTACCAT
GATTTGCTAAAAATTATTAAAGATAAAGATTTTTTGGATAA
TGAAGAAAATGAAGATATCTTAGAGGATATTGTTTTAACATT
GACCTTATTTGAAGATAGGGAGATGATTGAGGAAAGACTTA
AAACATATGCTCACCTCTTTGATGATAAGGTGATGAAACAGC
TTAAACGTCGCCGTTATACTGGTTGGGGACGTTTGTCTCGA
AAATTGATTAATGGTATTAGGGATAAGCAATCTGGCAAAACA
ATATTAGATTTTTTGAAATCAGATGGTTTTGCCAATCGCAA
TTTTATGCAGCTGATCCATGATGATAGTTTGACATTTAAAGA
AGACATTCAAAAAGCACAAGTGTCTGGACAAGGCGATAGTT
TACATGAACATATTGCAAATTTAGCTGGTAGCCCTGCTATTA
AAAAAGGTATTTTACAGACTGTAAAAGTTGTTGATGAATTG
GTCAAAGTAATGGGGCGGCATAAGCCAGAAAATATCGTTATT
GAAATGGCACGTGAAAATCAGACAACTCAAAAGGGCCAGAA
AAATTCGCGAGAGCGTATGAAACGAATCGAAGAAGGTATCAA
AGAATTAGGAAGTCAGATTCTTAAAGAGCATCCTGTTGAAA
ATACTCAATTGCAAAATGAAAAGCTCTATCTCTATTATCTCC
AAAATGGAAGAGACATGTATGTGGACCAAGAATTAGATATT
AATCGTTTAAGTGATTATGATGTCGATCACATTGTTCCACAA
AGTTTCCTTAAAGACGATTCAATAGACAATAAGGTCTTAAC
GCGTTCTGATAAAAATCGTGGTAAATCGGATAACGTTCCAAG
TGAAGAAGTAGTCAAAAAGATGAAAAACTATTGGAGACAAC
GCTGGTTTTATCAAACGCCAATTGGTTGAAACTCGCCAAATC
ACTAAGCATGTGGCACAAATTTTGGATAGTCGCATGAATAC
TAAATACGATGAAAATGATAAACTTATTCGAGAGGTTAAAGT
GATTACCTTAAAATCTAAATTAGTTTCTGACTTCCGAAAAG
ATTTCCAATTCTATAAAGTACGTGAGATTAACAATTACCATC
ATGCCCATGATGCGTATCTAAATGCCGTCGTTGGAACTGCT
TTGATTAAGAAATATCCAAAACTTGAATCGGAGTTTGTCTAT
GGTGATTATAAAGTTTATGATGTTCGTAAAATGATTGCTAA
GTCTGAGCAAGAAATAGGCAAAGCAACCGCAAAATATTTCTT
TTACTCTAATATCATGAACTTCTTCAAAACAGAAATTACAC
TTGCAAATGGAGAGATTCGCAAACGCCCTCTAATCGAAACTA
ATGGGGAAACTGGAGAAATTGTCTGGGATAAAGGGCGAGAT
TTTGCCACAGTGCGCAAAGTATTGTCCATGCCCCAAGTCAAT
ATTGTCAAGAAAACAGAAGTACAGACAGGCGGATTCTCCAA
GGAGTCAATTTTACCAAAAAGAAATTCGGACAAGCTTATTGC
TCGTAAAAAAGACTGGGATCCAAAAAAATATGGTGGTTTTG
ATAGTCCAACGGTAGCTTATTCAGTCCTAGTGGTTGCTAAGG
TGGAAAAAGGGAAATCGAAGAAGTTAAAATCCGTTAAAGAG
TTACTAGGGATCACAATTATGGAAAGAAGTTCCTTTGAAAAA
AATCCGATTGACTTTTTAGAAGCTAAAGGATATAAGGAAGT
TAAAAAAGACTTAATCATTAAACTACCTAAATATAGTCTTTT
TGAGTTAGAAAACGGTCGTAAACGGATGCTGGCTAGTGCCG
GAGAATTACAAAAAGGAAATGAGCTGGCTCTGCCAAGCAAAT
ATGTGAATTTTTTATATTTAGCTAGTCATTATGAAAAGTTG
AAGGGTAGTCCAGAAGATAACGAACAAAAACAATTGTTTGTG
GAGCAGCATAAGCATTATTTAGATGAGATTATTGAGCAAAT
CAGTGAATTTTCTAAGCGTGTTATTTTAGCAGATGCCAATTT
AGATAAAGTTCTTAGTGCATATAACAAACATAGAGACAAAC
CAATACGTGAACAAGCAGAAAATATTATTCATTTATTTACGT
TGACGAATCTTGGAGCTCCCGCTGCTTTTAAATATTTTGAT
ACAACAATTGATCGTAAACGATATACGTCTACAAAAGAAGTT
TTAGATGCCACTCTTATCCATCAATCCATCACTGGTCTTTA
TGAAACACGCATTGATTTGAGTCAGCTAGGAGGTGAC

1. An engineered cell comprising:
an endogenous gene or genomic region to be targeted for gene alteration, or one or more heterologous polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype; and
(a) a heterologous polynucleotide sequence comprising a promoter operably linked to a polynucleotide encoding a Cas nuclease and a heterologous polynucleotide sequence comprising a guide RNA (gRNA) that targets the endogenous gene, genomic region, or phenotype coding sequence; and/or (b) a heterologous polynucleotide sequence comprising a promoter operably linked to a polynucleotide encoding lambda red.

2. The engineered cell of claim 1, comprising:
an endogenous gene or genomic region to be targeted for gene alteration, or one or more heterologous polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype;
a heterologous polynucleotide sequence comprising a promoter operably linked to a polynucleotide encoding a Cas nuclease; and
a heterologous polynucleotide sequence comprising a gRNA that targets the endogenous gene, genomic region, or phenotype coding sequence.

3. The engineered cell of claim 1, comprising:
an endogenous gene or genomic region to be targeted for gene alteration, or one or more heterologous polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype;
a heterologous polynucleotide sequence comprising a promoter operably linked to a polynucleotide encoding lambda red; and
a heterologous polynucleotide sequence comprising a donor DNA sequence.

4. The engineered cell of claim 1, comprising a heterologous polynucleotide sequence comprising a promoter operably linked to a polynucleotide encoding lambda red, with or without the presence of a heterologous polynucleotide sequence comprising a promoter operably linked to a polynucleotide encoding a Cas nuclease and a heterologous polynucleotide sequence comprising a gRNA that targets the endogenous gene, genomic region, or phenotype coding sequence.

5. The engineered cell of claim 1, comprising a heterologous polynucleotide sequence comprising a promoter operably linked to a polynucleotide encoding a Cas nuclease and a heterologous polynucleotide sequence comprising a gRNA that targets the endogenous gene, genomic region, or phenotype coding sequence, with or without the presence of a heterologous polynucleotide sequence comprising a promoter operably linked to a polynucleotide encoding lambda red.

6. The engineered cell of claim 1, comprising:
an endogenous gene or genomic region to be targeted for gene alteration, or one or more heterologous polynucleotide sequences comprising a phenotype coding sequence operably linked to a promoter, wherein the phenotype coding sequence either (i) encodes a functional protein having a detectable phenotype, or (ii) comprises an engineered disruption in a sequence encoding a protein that prevents expression of the detectable phenotype;
a heterologous polynucleotide sequence comprising a promoter operably linked to a polynucleotide encoding a Cas nuclease;
a heterologous polynucleotide sequence comprising a gRNA that targets the endogenous gene, genomic region, or phenotype coding sequence;
a heterologous polynucleotide sequence comprising a promoter operably linked to a polynucleotide encoding lambda red; and
a heterologous polynucleotide sequence comprising a donor DNA sequence.

7. The engineered cell of claim 1, comprising an endogenous gene or genomic region to be targeted for gene alteration.

8. The engineered cell of claim 7, wherein:
the endogenous gene or genomic region to be targeted for gene alteration is a functional gene or genomic region and the donor DNA sequence is a sequence that disrupts the functional gene or genomic region; or
the endogenous gene or genomic region to be targeted for gene alteration is a functional gene or genomic region and the donor DNA sequence is a sequence that replaces the functional gene or genomic region with a different functional gene or different functional genomic region; or
the endogenous gene or genomic region to be targeted is a disrupted gene or genomic region and wherein the donor DNA sequence is a sequence that restores the function of the gene or genomic region.

9. The engineered cell of claim 1, comprising one or more heterologous polynucleotide sequences comprising a phenotype coding sequence operably linked to a promoter, wherein the phenotype coding sequence either (i) encodes a functional protein having a detectable phenotype.

10. The engineered cell of claim 1, comprising:
a heterologous polynucleotide sequence comprising a chromogenic coding sequence that encodes a functional chromogenic protein;
a heterologous polynucleotide sequence comprising a promoter operably linked to a polynucleotide encoding a Cas nuclease;
a heterologous polynucleotide sequence comprising a gRNA that targets the chromogenic coding sequence; and
a heterologous polynucleotide sequence comprising a homologous donor DNA sequence.

11. The engineered cell of claim 10, wherein the polynucleotide sequence comprising the gRNA that targets the chromogenic coding sequence is operably linked to the polynucleotide sequence comprising the promoter operably linked to the polynucleotide encoding a Cas nuclease.

12 The engineered cell of claim 10, wherein:
(i) the polynucleotide sequence comprising the gRNA that targets the chromogenic coding sequence is operably linked to the heterologous polynucleotide sequence comprising a homologous donor DNA sequence; or
(ii) the polynucleotide sequence comprising the gRNA that targets the chromogenic coding sequence and the heterologous polynucleotide sequence comprising a homologous donor DNA sequence are operably linked to the polynucleotide sequence comprising the promoter operably linked to the polynucleotide encoding a Cas nuclease.

13. The engineered cell of claim 10, wherein the heterologous polynucleotide sequence comprising the homologous donor DNA sequence comprises a mutation that prevents expression of the chromogenic protein.

14. The engineered cell of claim 10, wherein the heterologous polynucleotide sequence comprising the homologous donor DNA sequence comprises a mutation that modifies expression of the chromogenic protein from a first color to a second color.

15. The engineered cell of claim 1, comprising:
a heterologous polynucleotide sequence comprising an engineered disruption in a sequence encoding a chromogenic protein that prevents expression of the functional chromogenic protein;
a heterologous polynucleotide sequence comprising a promoter operably linked to a polynucleotide encoding a Cas nuclease;
a heterologous polynucleotide sequence comprising a gRNA that targets the polynucleotide sequence comprising the engineered disruption in the sequence encoding the chromogenic protein; and
a heterologous polynucleotide sequence comprising a homologous donor DNA sequence for repairing the engineered disruption.

16. The engineered cell of claim 1, comprising:
a first heterologous polynucleotide sequence comprising a first phenotype coding sequence operably linked to a first promoter, wherein the first phenotype coding sequence either (i) encodes a functional first protein having a first detectable phenotype, or (ii) comprises an engineered disruption in a sequence encoding a first protein that prevents expression of the first detectable phenotype;
a second heterologous polynucleotide sequence comprising a second phenotype coding sequence operably linked to a second promoter, wherein the second phenotype coding sequence either (i) encodes a functional second protein having a second detectable phenotype, or (ii) comprises an engineered disruption in a sequence encoding a second protein that prevents expression of the second detectable phenotype;
a third heterologous polynucleotide sequence comprising a third promoter operably linked to a polynucleotide encoding a Cas nuclease;
a fourth heterologous polynucleotide sequence comprising a first gRNA that targets the first phenotype coding sequence; and
a fifth heterologous polynucleotide sequence comprising a second gRNA that targets the second phenotype coding sequence.

17. (canceled)

18. The engineered cell of claim 1, wherein the detectable phenotype is a detectable fluorescence, and wherein the phenotype coding sequence either (i) encodes a functional fluorescent protein, or (ii) comprises an engineered disruption in a sequence encoding a protein that prevents expression of the fluorescent protein.

19. (canceled)

20. The engineered cell of claim 16, wherein the first detectable phenotype and the second detectable phenotype is a detectable color and/or fluorescence, and wherein:
the first chromogenic coding sequence either (i) encodes a functional first chromogenic protein or (ii) comprises an engineered disruption in a sequence encoding a first chromogenic protein that prevents expression of the first chromogenic protein; and
the second chromogenic coding sequence either (i) encodes a functional second chromogenic protein or (ii) comprises an engineered disruption in a sequence encoding a second chromogenic protein that prevents expression of the second chromogenic protein.

21. (canceled)

22. The engineered cell of claim 16, wherein the fourth polynucleotide sequence further comprises a first auxotrophic, antibiotic, or other selectable marker and the fifth polynucleotide sequence further comprises a second auxotrophic, antibiotic, or other selectable marker, wherein the first auxotrophic, antibiotic, or other selectable marker and the second auxotrophic, antibiotic, or other selectable marker are different markers.

23. The engineered cell of claim 1, wherein the polynucleotide sequence comprising a gRNA further comprises a homologous donor polynucleotide sequence.

24.-26. (canceled)

27. The engineered cell of claim 1, wherein the Cas nuclease is a Cas9 nuclease or a destabilized variant of Cas9.

28. (canceled)

29. The engineered cell of claim 1, wherein:
the cell is a yeast cell and the polynucleotide encoding the Cas nuclease encodes a Cas9 nuclease that is codon optimized for expression in yeast; or the cell is a bacterial cell and the polynucleotide encoding the Cas nuclease encodes a Cas9 nuclease that is codon optimized for expression in bacteria.

30.-40. (canceled)

41. An engineered microbial organism comprising the engineered cell of claim 1.

42. (canceled)

43. A kit comprising:
the engineered cell of claim 1; and
one or more reagents comprising culture medium, selective medium, media supplements, solid plating medium, plates, tubes, loops, or other plasticware.

44.-46. (canceled)

47. A method of altering gene expression in a cell, the method comprising:
culturing the engineered cell of claim 1 to form a population of engineered cells, wherein the culturing is performed under conditions that result in expression of the Cas nuclease in at least one engineered cell, wherein the Cas nuclease cleaves an endogenous gene, genomic region, or phenotype coding sequence in the engineered cell;
thereby altering gene expression in at least one engineered cell.

48.-71. (canceled)

72. A modular system for training an individual in laboratory procedures and the use of laboratory equipment for gene expression and gene editing, the modular system comprising:
(a) a module for expressing a detectable phenotype and/or for expressing a broken gene in a cell or organism, comprising the engineered cell of claim 1 and further comprising one or more reagents for culturing reagents, transfecting, and/or transforming the cell or organism;
(b) a module for altering expression of the detectable phenotype and/or for repairing the broken gene in the cell or organism, comprising one or more reagents for expressing a Cas nuclease, expressing a gRNA, or preventing expression of a gRNA in the cell or organism;
(c) a module for analyzing the cell or organism, comprising one or more reagents for detecting the alteration of gene expression and/or repair of the broken gene in the cell or organism; and
(d) an instruction manual, comprising instructions for use of each of the modules.

73. (canceled)


SEQUENCE LISTING (5 sequences)

SEQ ID NO: 1 - DNA, 4182 bp, Artificial Sequence
  Description of Artificial Sequence Synthetic polynucleotide
atgtatccatatgatgttccagattacgctccacctaagaagaaacgtaaggttgacaaaaagtactccatcggtttagatattggtaccaactctgtcggttgggccgttattactgatgaatacaaggttccatctaagaagttcaaagttttaggtaacactgatagacactccattaagaagaatttgatcggtgctttgttgttcgattccggtgaaaccgccgaagctaccagattaaagagaaccgctagaagaagatataccagaagaaagaacagaatttgttacttgcaagaaattttctccaacgaaatggccaaggttgatgattctttctttcacagattggaagaatcctttttagtcgaagaagataagaaacacgaaagacacccaatcttcggtaacattgtcgacgaagtcgcttatcacgaaaaatatccaactatttaccacttgagaaagaagttagtcgactccacctacaaagctgacttgagattgatttatttggctttggctcacatgattaagttcagaggtcacttcttaatcgaaggtgacttgaaccctgacaattccgatgttgacaagttgttcatccaattggtccaaacctataatcaattgttcgaagaaaatccaatcaacgcttccggtgttgacgctaaagccatcttgtctgccagattgtccaagtcccgtcgtttagaaaacttgattgctcaattgccaggtgaaaagaagaacggtttgtttggtaacttgattgctttgtccttgggtttaaccccaaacttcaagtctaacttcgatttggccgaagatgctaagttgcaattgtccaaggatacttacgatgacgatttggataacttattggcccaaatcggtgaccaatacgctgacttgttcttggccgctaagaacttatccgacgccatcttgttgtccgacattttaagagttaacactgaaattaccaaggccccattgtccgcctccatgatcaagagatacgacgaacaccaccaagacttgaccttattgaaggctttagttcgtcaacaattaccagaaaagtataaagaaatcttctttgatcaatctaaaaacggttacgctggttatattgatggtggtgcctctcaagaagaattctacaaatttatcaagcctatcttagaaaaaatggacggtaccgaagaattattggtcaagttaaacagagaagatttgttgcgtaagcaacgtactttcgacaacggttccatcacccatcaaatccacttgggtgaattgcacgctatcttaagaagacaagaagatttctacccattcttgaaagataatagagaaaaaattgaaaaaattttgactttcagaattccttactacgttggtccattagccagaggtaactctagatttgcttggatgactagaaagtccgaagaaactattaccccatggaacttcgaagaagtcgttgacaagggtgcttccgctcaatccttcattgaaagaatgaccaatttcgataaaaacttaccaaacgaaaaggttttgccaaagcactctttgttatatgaatacttcaccgtttacaacgaattgactaaagtcaagtacgttaccgaaggtatgagaaagccagctttcttgtctggtgagcaaaagaaggctattgttgacttattattcaaaactaacagaaaggtcactgtcaagcaattgaaggaagattatttcaaaaagatcgaatgcttcgactctgttgaaatctctggtgttgaagatagattcaacgcttccttgggtacctatcacgatttgttgaaaatcatcaaggacaaggactttttggataacgaggaaaacgaagacattttagaagatattgttttgactttgaccttgttcgaagacagagaaatgatcgaagaaagattgaagacctacgctcatttgttcgacgataaagtcatgaaacaattgaagagaagaagatataccggttggggtagattatctagaaagttaattaatggtattagagacaagcaatccggtaagaccatcttggatttcttaaaatctgacggtttcgctaaccgtaacttcatgcaattgattcatgatgactctttgaccttcaaagaagatattcaaaaggctcaagtctctggtcaaggtgattctttgcacgaacatattgctaacttggctggttcccctgccattaagaagggtattttgcaaactgttaaggtcgttgacgaattggtcaaggttatgggtagacacaaaccagaaaacatcgtcattgaaatggccagagaaaaccaaaccacccaaaaaggtcaaaagaactcccgtgaaagaatgaagagaatcgaagaaggtatcaaggagttgggttctcaaattttaaaggaacatccagtcgagaacactcaattgcaaaacgaaaagttgtacttgtactacttacaaaacggtagagatatgtacgtcgatcaagaattagacattaatagattgtctgactacgacgtcgaccatattgtcccacaatctttcttaaaggacgattccattgacaacaaagttttaactagatccgacaaaaacagaggtaagtctgataacgttccttctgaagaagtcgtcaagaagatgaagaactactggagacaattgttgaacgccaaattgatcacccaaagaaagttcgacaacttaaccaaggctgaaagaggtggtttgtctgaattggataaggctggttttattaagagacaattggtcgaaaccagacaaattactaaacatgttgctcaaatcttggattctcgtatgaataccaaatacgacgaaaacgataaattgattagagaagtcaaggttattactttgaagtccaagttggtttctgatttccgtaaggacttccaattctacaaagttagagaaattaacaattaccaccacgctcatgacgcttacttgaacgctgtcgttggtactgccttgattaagaagtacccaaaattggaatccgaattcgtttatggtgactacaaagtttacgatgtcagaaaaatgattgctaagtctgaacaagagattggtaaagctactgccaaatatttcttttactctaacatcatgaacttctttaagaccgaaatcactttagctaacggtgaaattcgtaagagaccattaatcgaaactaacggtgaaactggtgaaattgtctgggataagggtagagatttcgccaccgttcgtaaggttttgtctatgcctcaagttaatatcgtcaagaagaccgaagtccaaaccggtggtttttctaaggaatctatcttgccaaagagaaattctgacaagttgattgctagaaagaaagactgggatccaaagaagtacggtggttttgactccccaactgttgcttactccgttttggttgttgctaaggttgaaaagggtaagtctaagaagttaaagtctgttaaggaattgttgggtattaccattatggaaagatcttcttttgagaaaaacccaattgactttttagaggctaagggttacaaggaagttaagaaggacttgatcattaaattgccaaagtattctttgttcgaattggaaaacggtcgtaagcgtatgttggcctctgctggtgaattacaaaagggtaacgaattagctttgccttctaaatacgttaactttttatacttggcttcccattacgaaaagttaaaaggttctccagaagacaatgaacaaaaacaattgttcgttgaacaacacaagcattacttggacgaaattattgaacaaatttctgaattttccaagagagttatcttagccgacgctaacttggacaaggtcttgtctgcttacaataagcatagagacaagccaatccgtgaacaagccgaaaacatcattcacttgttcactttgactaacttgggtgctccagctgccttcaagtacttcgacaccactatcgacagaaagagatacacttctactaaggaggttttagatgccaccttgattcaccaatctattactggtttgtacgagactagaattgatttgtcccaattaggtggtgatccacctaagaagaagagaaaggtttaa

SEQ ID NO: 2 - DNA, 4134 bp, Artificial Sequence
  Description of Artificial Sequence Synthetic polynucleotide
atgtatccatacgacgtgcctgactatgcggacaaaaagtactccatcggtttggacatcgggacgaatagcgttgggtgggcggtgattacagatgaatataaggtgcctagtaaaaaattcaaagtattaggcaataccgatcgtcatagcattaagaagaacctgattggagcattgctttttgattcgggtgaaaccgcagaagcaacgcgtttgaagcgcacagcacgtcgtcgctacacacgccgcaaaaatcgtatttgttatcttcaagaaattttttcaaatgagatggcaaaggtcgacgattcttttttccatcgtttagaggaatcttttcttgtggaggaagataagaagcacgagcgtcacccaatctttggtaatattgtcgacgaagttgcgtaccatgaaaagtacccaacgatctaccacctgcgtaagaaattggtggactcgacatacaaggcggatcttcgcttgatctatcttgctttggcccacatgattaagttccgcgggcatttcttaattgaaggagacttaaacccggataactcagatgttgacaagctttttattcagcttgtgcaaacttacaatcaacttttcgaagaaaaccctatcaacgcctctggtgtggatgcgaaggcgatcctttcggcgcgcctgtcaaagagtcgtcgcttggagaatttgattgcgcagttgccgggggagaagaaaaatggcctgtttggaaacctgattgcgctttctcttggattaactcccaactttaagtcaaacttcgacttggctgaggatgccaagttacagctgtccaaagatacctacgatgatgatcttgataacttgctggctcaaatcggtgaccaatacgcggatcttttcttggccgcgaagaacttgtctgacgcaatccttttatcggacatcctgcgcgttaacacagagatcactaaagcccctctgtctgcatcaatgattaaacgctacgacgaacaccatcaggatttgacattactgaaagcccttgtacgtcaacaacttccggaaaagtacaaagaaatcttcttcgatcagtctaaaaacgggtacgccgggtatattgatggtggcgcttcacaggaggagttttacaaattcattaaacctattctggagaaaatggatggaacggaagagttgttggtgaagcttaatcgtgaagaccttttgcgcaagcagcgtacgttcgataacgggtcaatcacacaccaaatccacttgggcgagttacatgcaattcttcgccgtcaggaagatttctaccctttcttgaaagataaccgcgagaagattgaaaagattttgacttttcgtatcccctactacgtgggtcctttagcccgtggaaacagtcgcttcgcctggatgacccgcaagtcagaagaaacgatcaccccctggaattttgaggaggttgtggataagggggcgtcagcgcaaagcttcatcgaacgcatgacgaacttcgataagaacttacctaatgagaaagtgctgccaaaacatagtcttctttatgagtacttcactgtttacaatgagttaactaaggtaaagtatgttacagaagggatgcgtaaacccgcatttttatccggtgagcaaaagaaagctatcgtggatttgttatttaagactaaccgcaaagtaacagtcaaacaattaaaagaagactactttaagaaaattgagtgctttgactcagtggagatctctggtgtcgaggaccgctttaatgcctcattgggaacttatcacgacttactgaagattattaaagataaagactttcttgacaacgaagagaacgaagatattctggaggacatcgtcttaacactgactctgttcgaggatcgcgagatgattgaggaacgcttgaaaacttatgcccacttatttgatgacaaagtgatgaaacaacttaaacgtcgtcgctacaccggatggggtcgtttatcacgtaaattaatcaacggcattcgcgataagcagtccggcaaaacaatccttgattttctgaagtccgacggattcgcgaatcgcaactttatgcagctgatccacgatgatagtcttaccttcaaggaggatattcaaaaagcccaggtatcggggcaaggtgactccctgcacgaacatattgcgaatttggccgggtctccggcaatcaagaaaggtattttacagaccgttaaagtcgtggatgaattggttaaggtaatgggccgtcataaaccggagaatatcgtaattgaaatggcgcgtgagaatcagacaactcagaaaggacaaaagaatagccgcgaacgtatgaaacgtattgaagagggaatcaaagaattagggagtcagatcttaaaagaacatccagttgaaaacacccaattgcaaaacgaaaagttatacttatactaccttcagaacggccgcgatatgtacgtagatcaggaattagacattaaccgcctgtcagattacgatgtcgaccatattgttcctcagtctttcttaaaggacgatagcatcgataataaagttttaactcgttcggataaaaaccgtggaaaatccgacaacgtcccatctgaggaggtagtcaagaagatgaagaactactggcgtcagttactgaacgcgaaattgatcactcagcgcaaatttgataatttgactaaagccgaacgcggtggtttgtcggagttagacaaagccggcttcatcaaacgccagcttgtagagacccgccagattacgaagcacgttgcccagattcttgacagccgtatgaacaccaagtacgatgagaatgataaactgattcgcgaggtgaaggtaatcacgttaaagagcaaactggtaagtgattttcgtaaggactttcaattttacaaagtgcgcgagatcaacaactatcaccatgcgcatgatgcctatttgaatgccgtagtcggtacagctttgattaagaagtatcctaagttggagtcagaatttgtctacggcgactacaaggtgtatgacgtacgcaagatgattgcgaagtccgagcaggaaattggcaaggccactgctaagtacttcttttattctaacattatgaacttcttcaaaaccgaaatcacccttgcgaacggggaaattcgtaagcgcccgttgatcgaaacaaatggcgagactggtgaaattgtttgggacaaaggtcgcgattttgcaacggtgcgtaaagtattaagtatgccccaggtaaatattgtcaagaagacggaagtgcaaaccggtgggttttctaaagaatcaattttgccgaagcgtaattctgataaattaattgcgcgcaaaaaggactgggaccctaagaaatacggcgggttcgactcacccacggtcgcctattccgtgttagttgttgcgaaagtcgagaaaggtaaaagcaagaaacttaagtctgttaaggaattgttaggaatcacgattatggaacgtagctcatttgagaaaaaccccattgactttttggaggcgaaaggttacaaggaagttaaaaaagacctgattattaaacttcccaagtacagccttttcgaactggaaaatggtcgtaagcgcatgctggcgtcggccggtgaactgcaaaaggggaatgagttggccctgccatcgaagtatgtgaatttcttatacctggcgtcccattacgaaaaattaaaaggctcaccggaagataatgagcaaaagcagctgtttgtggagcaacacaaacattacttagatgaaattatcgaacagatttcggagttcagcaagcgcgtgattcttgcggacgcgaatcttgataaggttttatctgcttacaacaagcaccgcgacaagccgatccgtgagcaagctgagaacattattcatttattcacactgactaatcttggggcgccagctgcctttaaatactttgacaccaccattgaccgcaaacgctacacaagtacaaaagaagtcttagacgcgacactgattcatcaatccatcactggtttatatgaaacacgcatcgatctttcgcaactgggaggtgattaa

SEQ ID NO: 3 - DNA, 4104 bp, Streptococcus pyogenes
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac

SEQ ID NO: 4 - DNA, 29 bp, Escherichia coli
gggtcggttacggccaggacagtcgtttg

SEQ ID NO: 5 - DNA, 26 bp, Escherichia coli
gggtcgcggccgcttaattaagtttg